(19) United States
(12) Patent Application Publication
(10) Pub. No.: US 2023/0015767 A1
(43) Pub. Date: Jan. 19, 2023

(54) PHOTOSENSITIVE COLORING COMPOSITION, CURED SUBSTANCE, COLOR FILTER, SOLID-STATE IMAGING ELEMENT, IMAGE DISPLAY DEVICE, AND ASYMMETRIC DIKETOPYRROLOPYRROLE COMPOUND

(71) Applicant: FUJIFILM Corporation, Tokyo (JP)

(72) Inventors: Ryosuke KATO, Shizuoka (JP); Yushi KANEKO, Shizuoka (JP); Junichi ITO, Shizuoka (JP)

(73) Assignee: FUJIFILM Corporation, Tokyo (JP)

(21) Appl. No.: 17/895,309

(22) Filed: Aug. 25, 2022

Related U.S. Application Data
(63) Continuation of application No. PCT/JP2021/005999, filed on Feb. 17, 2021.

(30) Foreign Application Priority Data
Feb. 26, 2020 (JP) 2020-030706

(51) Int. Cl. G03F 7/004; G03F 7/00; G03F 7/028

(52) U.S. Cl. CPC G03F 7/0045; G03F 7/0007; G03F 7/028; G02B 5/223

(57) ABSTRACT

Provided are a photosensitive coloring composition including a pigment and a diketopyrrolopyrrole compound A represented by Formula 1, in which, in a case where molar contents of the diketopyrrolopyrrole compound A and a diketopyrrolopyrrole compound B represented by Formula 1 are each denoted by m^{A} and m^{B}, a value of m^{A}/(m^{A}+m^{B}) is 10 mol % to 100 mol %, and a content of the pigment is 35% by mass or more with respect to a total solid content of the photosensitive coloring composition; a cured substance of the photosensitive coloring composition, a color filter including the cured substance, a solid-state imaging element or an image display device, and a novel asymmetric diketopyrrolopyrrole compound.

Diketopyrrolopyrrole compound A: A¹ represents a monovalent organic group having an acidic or a basic functional group, and B¹ represents a monovalent organic group not having an acidic or a basic functional group.

Diketopyrrolopyrrole compound B: A¹ and B¹ represent monovalent organic groups having an acidic or a basic functional group.

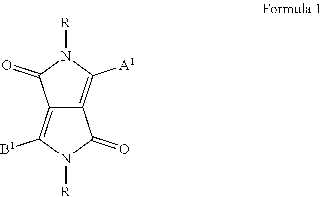

### CROSS-REFERENCE TO RELATED APPLICATIONS

This application is a Continuation of International Application No. PCT/JP2021/005999 filed on Feb. 17, 2021, which claims priority to Japanese Patent Application No. 2020-030706 filed on Feb. 26, 2020. The entire contents of these applications are incorporated herein by reference.

### BACKGROUND OF THE INVENTION

### 1. Field of the Invention

The present disclosure relates to a photosensitive coloring composition, a cured substance, a color filter, a solid-state imaging element, an image display device, and an asymmetric diketopyrrolopyrrole compound.

### 2. Description of the Related Art

A color filter is an indispensable component for a solid-state imaging element and an image display device. The solid-state imaging element and the image display device may generate noise due to the reflection of visible light. Therefore, a light shielding film may be provided on the solid-state imaging element or the image display device to suppress the generation of noise.

As a method for producing such a color filter or a light shielding film, a method in which a photosensitive coloring composition layer is formed using a photosensitive coloring composition including a colorant, a polymerizable compound, a photopolymerization initiator, and an alkali-soluble resin, and the photosensitive coloring composition layer is exposed and developed to form a pattern has been known.

As a photosensitive coloring composition or photosensitive composition in the related art, compositions disclosed in JP2013-182230A, WO2018/159541A, and JP2011-523433B have been known.

### SUMMARY OF THE INVENTION

An object to be achieved by the embodiment according to the present disclosure is to provide a photosensitive coloring composition having excellent adhesiveness of a cured substance to be obtained.

Another object to be achieved by the embodiment according to the present disclosure is to provide a cured substance of the above-described photosensitive coloring composition, a color filter including the cured product, and a solid-state imaging element or an image display device including the color filter.

Another object to be achieved by the embodiment according to the present disclosure is to provide a novel asymmetric diketopyrrolopyrrole compound.

The methods for achieving the above-described objects include the following aspects.

<1> A photosensitive coloring composition comprising:

a pigment; and

a diketopyrrolopyrrole compound A represented by Formula 1,

in which, in a case where a molar content of the diketopyrrolopyrrole compound A represented by Formula 1 in the photosensitive coloring composition is denoted by m^{A} and a molar content of a diketopyrrolopyrrole compound B represented by Formula 1 is denoted by m^{B}, a value of m^{A}/(m^{A}+m^{B}) is 10 mol % to 100 mol %, and

a content of the pigment is 35% by mass or more with respect to a total solid content of the photosensitive coloring composition.

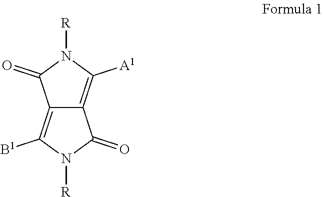

In Formula 1,

diketopyrrolopyrrole compound A: A¹ represents a monovalent organic group having an acidic functional group or a basic functional group, B¹ represents a monovalent organic group not having an acidic functional group and a basic functional group, and R's each independently represent a hydrogen atom or a monovalent substituent,

diketopyrrolopyrrole compound B: A¹ and B¹ represent monovalent organic groups having an acidic functional group or a basic functional group, A¹ and B¹ may be the same or different from each other, and R's each independently represent a hydrogen atom or a monovalent substituent.

<2> The photosensitive coloring composition according to <1>,

in which the diketopyrrolopyrrole compound A includes an asymmetric diketopyrrolopyrrole compound represented by Formula 2.

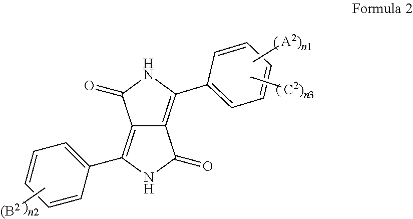

In Formula 2, A²'s each independently represent a monovalent organic group having an acidic functional group or a basic functional group, B²'s each independently represent a monovalent organic group not having an acidic functional group and a basic functional group, C²'s each independently represent a monovalent organic group not having an acidic functional group and a basic functional group, n1 represents an integer of 1 to 5, n2 represents an integer of 0 to 5, n3 represents an integer of 0 to 4, and a phenyl group to which A² and C² are bonded and a phenyl group to which B² is bonded are different groups.

<3> The photosensitive coloring composition according to <1> or <2>,

in which the value of m^{A}/(m^{A}+m^{B}) is more than 90 mol % and 100 mol % or less.

<4> The photosensitive coloring composition according to <1>,

in which A¹ is a monovalent organic group having a basic functional group.

<5> The photosensitive coloring composition according to any one of <1> to <4>,

in which the pigment includes a diketopyrrolopyrrole pigment other than the compound represented by Formula 1.

<6> The photosensitive coloring composition according to any one of <1> to <5>,

in which the pigment includes a diketopyrrolopyrrole red pigment other than the compound represented by Formula 1.

<7> The photosensitive coloring composition according to any one of <1> to <6>,

in which the pigment includes a diaryldiketopyrrolopyrrole red pigment other than the compound represented by Formula 1, which has an electron donating group on an aromatic ring.

<8> The photosensitive coloring composition according to any one of <1> to <7>,

in which the content of the pigment is 50% by mass or more with respect to the total solid content of the photosensitive coloring composition.

<9> The photosensitive coloring composition according to any one of <1> to <8>,

in which a mass ratio of a content M^{P} of the pigment and a content M^{A} of the diketopyrrolopyrrole compound A in the photosensitive coloring composition is M^{P}/M^{A}=95/5 to 50/50.

<10> The photosensitive coloring composition according to any one of <1> to <9>, further comprising:

a resin.

<11> The photosensitive coloring composition according to <10>,

in which the resin includes a resin having an acidic functional group.

<12> The photosensitive coloring composition according to any one of <1> to <11>, further comprising:

a polymerizable compound; and

a photopolymerization initiator.

<13> A cured substance obtained by curing the photosensitive coloring composition according to any one of <1> to <12>.

<14> A color filter comprising:

the cured substance according to <13>.

<15> A solid-state imaging element comprising:

the color filter according to <14>.

<16> An image display device comprising:

the color filter according to <14>.

<17> An asymmetric diketopyrrolopyrrole compound represented by Formula 3.

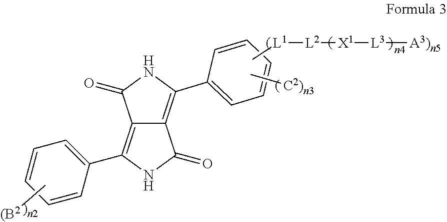

In Formula 3, A³'s each independently represent an acidic functional group or a basic functional group, B²'s each independently represent a monovalent organic group not having an acidic functional group and a basic functional group, C²'s each independently represent a monovalent organic group not having an acidic functional group and a basic functional group, X¹'s each independently represent an ether bond, a thioether bond, a sulfonamide bond, or a urea bond, L¹'s each independently represent a single bond or an ether bond, L²'s and L³'s each independently represent an alkylene group, n2 represents an integer of 0 to 5, n3 represents an integer of 0 to 4, n4's each independently represent 0 or 1, n5 represents an integer of 1 to 5, a group having A³ at a terminal, a phenyl group to which C² is bonded, and a phenyl group to which B² is bonded are different groups, and in a case where L¹ is an ether bond, B² is an electron donating group not having an acidic functional group and a basic functional group and n2 represents an integer of 1 to 5.

According to the embodiment according to the present disclosure, a photosensitive coloring composition having excellent adhesiveness of a cured substance to be obtained is provided.

According to another embodiment according to the present disclosure, a cured substance of the above-described photosensitive coloring composition, a color filter including the cured product, and a solid-state imaging element or an image display device including the color filter are provided.

According to another embodiment according to the present disclosure, a novel asymmetric diketopyrrolopyrrole compound is provided.

### DESCRIPTION OF THE PREFERRED EMBODIMENTS

Hereinafter, the content of the present disclosure will be described in detail. The configuration requirements will be described below based on the representative embodiments of the present disclosure, but the present disclosure is not limited to such embodiments.

In the present disclosure, a term “to” showing a range of numerical values is used as a meaning including a lower limit value and an upper limit value disclosed before and after the term.

In a range of numerical values described in stages in the present disclosure, the upper limit value or the lower limit value described in one range of numerical values may be replaced with an upper limit value or a lower limit value of the range of numerical values described in other stages. In addition, in a range of numerical values described in the present disclosure, the upper limit value or the lower limit value of the range of numerical values may be replaced with values shown in the examples.

Further, in the present disclosure, in a case where a plurality of substances corresponding to each component in a composition is present, the amount of each component in the composition means the total amount of the plurality of substances present in the composition, unless otherwise specified.

In addition, regarding a term, group (atomic group) of this present disclosure, a term with no description of “substituted” and “unsubstituted” includes both a group not including a substituent and a group including a substituent. For example, an “alkyl group” includes not only an alkyl group having no substituent (unsubstituted alkyl group), but also an alkyl group having a substituent (substituted alkyl group).

In the present disclosure, unless otherwise specified, “Me” represents a methyl group, “Et” represents an ethyl group, “Pr” represents a propyl group, “Bu” represents a butyl group, and “Ph” represents a phenyl group.

In the present disclosure, the concept of “(meth)acryl” includes both acryl and methacryl, and the concept of “(meth)acryloyl” includes both acryloyl and methacryloyl.

In addition, in the present disclosure, a term “step” includes not only the independent step but also a step in which intended purposes are achieved even in a case where the step cannot be precisely distinguished from other steps.

In the present disclosure, a “total solid content” refers to a total mass of components obtained by removing a solvent from the whole composition of the composition. In addition, a “solid content” is a component obtained by removing a solvent as described above, and for example, the component may be solid or may be liquid at 25° C.

In addition, in the present disclosure, “% by mass” is identical to “% by mass” and “part by mass” is identical to “part by weight”.

Furthermore, in the present disclosure, a combination of two or more preferred aspects is a more preferred aspect.

In addition, a weight-average molecular weight (Mw) and a number-average molecular weight (Mn) in the present disclosure are molecular weights in terms of polystyrene used as a standard substance, which are detected by using a solvent tetrahydrofuran (THF), a differential refractometer, and a gel permeation chromatography (GPC) analysis apparatus using TSKgel GMHxL, TSKgel G4000HxL, and TSKgel G2000HxL (all trade names manufactured by Tosoh Corporation) as columns, unless otherwise specified.

In the present specification, a pigment means a compound which is hardly dissolved in a solvent.

In the present specification, a dye means a compound which is easily soluble in a solvent.

Hereinafter, the present disclosure will be described in detail.

(Photosensitive Coloring Composition)

A photosensitive coloring composition according to an embodiment of the present disclosure includes a pigment and a diketopyrrolopyrrole compound A represented by Formula 1, in which, in a case where a molar content of the diketopyrrolopyrrole compound A represented by Formula 1 in the photosensitive coloring composition is denoted by m^{A} and a molar content of a diketopyrrolopyrrole compound B represented by Formula 1 is denoted by m^{B}, a value of m^{A}/(m^{A}+m^{B}) is 10 mol % to 100 mol %, and a content of the pigment is 35% by mass or more with respect to a total solid content of the photosensitive coloring composition.

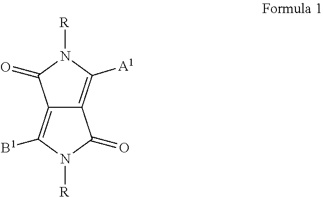

In Formula 1,

diketopyrrolopyrrole compound A: A¹ represents a monovalent organic group having an acidic functional group or a basic functional group, B¹ represents a monovalent organic group not having an acidic functional group and a basic functional group, and R's each independently represent a hydrogen atom or a monovalent substituent,

diketopyrrolopyrrole compound B: A¹ and B¹ represent monovalent organic groups having an acidic functional group or a basic functional group, A¹ and B¹ may be the same or different from each other, and R's each independently represent a hydrogen atom or a monovalent substituent.

In recent years, as the number of pixels of an image sensor has increased, a pattern has been finer and thinner. Along with this, a concentration of a pigment in a color filter increases relatively, and an amount of curable components is reduced. As a result of detailed studies, the present inventors have found that, in a photosensitive coloring composition in the related art in which the content of the pigment is 35% by mass or more with respect to the total solid content of the photosensitive coloring composition, an adhesiveness of a cured substance to be obtained to a substrate or the like may not be sufficient.

As a result of intensive studies, the present inventors have found that, by adopting the above-described configuration, the adhesiveness of the cured substance to be obtained is excellent.

Since the content of the pigment is 35% by mass or more with respect to the total solid content of the photosensitive coloring composition, and the photosensitive coloring composition includes the diketopyrrolopyrrole compound A represented by Formula 1 described, in which, in a case where the molar content of the diketopyrrolopyrrole compound A represented by Formula 1 in the photosensitive coloring composition is denoted by m^{A} and the molar content of the diketopyrrolopyrrole compound B represented by Formula 1 is denoted by m^{B}, the value of m^{A}/(m^{A}+m^{B}) is 10 mol % to 100 mol %, even in the above-described photosensitive coloring composition having a high pigment concentration, the above-described diketopyrrolopyrrole compound A is adsorbed on a surface of the pigment. In addition, in a case where a structure of pigment particles-asymmetric diketopyrrolopyrrole compound A is formed, compared to a symmetric diketopyrrolopyrrole compound, the acidic functional group or the basic functional group is likely to be disposed on a side opposite to the pigment side, and easily interacts with other components such as a dispersant. Therefore, since the diketopyrrolopyrrole ring structure has excellent adsorptivity to the pigment and the diketopyrrolopyrrole compound A is easily disposed on the surface of pigment particles, an interaction between the pigment particles and the above-described diketopyrrolopyrrole compound A and an interaction between other components and the above-described diketopyrrolopyrrole compound A are improved, and it is presumed that the cured substance to be obtained has excellent adhesiveness (hereinafter, also simply referred to as an “adhesiveness”).

<Diketopyrrolopyrrole Compounds A and B>

The photosensitive coloring composition according to the embodiment of the present disclosure includes a diketopyrrolopyrrole compound A represented by Formula 1 described above, in which, in a case where a molar content of the diketopyrrolopyrrole compound A represented by Formula 1 in the photosensitive coloring composition is denoted by m^{A} and a molar content of a diketopyrrolopyrrole compound B represented by Formula 1 is denoted by m^{B}, a value of m^{A}/(m^{A}+m^{B}) is 10 mol % to 100 mol %.

In the present disclosure, the above-described pigment is a pigment other than the diketopyrrolopyrrole compounds A and B represented by Formula 1 described above.

From the viewpoint of the adhesiveness and storage stability, the above-described value m^{A}/(m^{A}+m^{B}) in the present disclosure is preferably 50 mol % to 100 mol %, more preferably 80 mol % to 100 mol %, and particularly preferably more than 90 mol % and 100 mol % or less.

Further, in the photosensitive coloring composition according to the embodiment of the present disclosure, in a case where a content of an isomer having the highest content among the diketopyrrolopyrrole compound A represented by Formula 1 described above is denoted by m^{AA}, from the viewpoint of the adhesiveness and storage stability, the above-described value m^{A}/m^{A} in the present disclosure is preferably 80 mol % to 100 mol %, more preferably 90 mol % to 100 mol %, and particularly preferably 95 mol % to 100 mol %.

As the acidic functional group in A¹ and B¹ of Formula 1, from the viewpoint of the adhesiveness and storage stability, a sulfo group, a salt of a sulfo group, a carboxy group, a phosphorus acidic functional group, a hydroxy group, or a boroacidic functional group is preferable, and a sulfo group, a salt of a sulfo group, or a carboxy group is more preferable.

From the viewpoint of the adhesiveness and storage stability, a counter ion of the salt of a sulfo group is preferably a metal ion, a monoalkylammonium ion having 1 to 12 carbon atoms, a dialkylammonium ion having 2 to 24 carbon atoms, a trialkylammonium ion having 3 to 36 carbon atoms, or a tetraalkylammonium ion having 4 to 48 carbon atoms, more preferably a metal ion, a monoalkylammonium ion having 1 to 12 carbon atoms, a dialkylammonium ion having 2 to 24 carbon atoms, or a trialkylammonium ion having 3 to 36 carbon atoms, still more preferably a metal ion, and particularly preferably an alkali metal ion.

As the basic functional group in A¹ and B¹ of Formula 1, from the viewpoint of the adhesiveness and storage stability, a group having a nitrogen atom is preferable, —NR^{1A}R^{2A} or a heterocyclic group including a nitrogen atom is more preferable, a piperidinyl group, a morpholinyl group, or —NR^{1A}R^{2A} is still more preferable, and —NR^{1A}R^{2A} is particularly preferable.

R^{1A} and R^{2A} in —NR^{1A}R^{2A} are each independently preferably a hydrogen atom, an alkyl group, an aryl group, or a heteroaryl group, more preferably an alkyl group, an aryl group, or a heteroaryl group, still more preferably an alkyl group, and particularly preferably an alkyl group having 1 to 4 carbon atoms.

The alkyl group in R^{1A} and R^{2A} may have a substituent, and examples thereof include a halogen atom, an aryl group, a heteroaryl group, an alkoxy group, an alkylthio group, and a dialkylamino group.

The aryl group and heteroaryl group in R^{1A} and R^{2A} may have a substituent, and examples thereof include a halogen atom, an alkyl group, an aryl group, a heteroaryl group, an alkoxy group, an alkylthio group, and a dialkylamino group.

In addition, R^{1A} and R^{2A} in —NR^{1A}R^{2A} may be bonded to each other to form a ring, and it is preferable to form a 5-membered ring or a 6-membered ring and more preferable to form a 6-membered ring.

The ring formed by bonding R^{1A} and R^{2A} to each other may have, as a ring member, a heteroatom other than the nitrogen atom to which R^{1A} and R^{2A} are bonded, or may have a substituent. Examples of the substituent which may be included in the ring include a halogen atom, an alkyl group, an aryl group, a heteroaryl group, an alkoxy group, and an alkylthio group.

As the heterocyclic group including the nitrogen atom, an imidazolyl group, a pyrazolyl group, a triazolyl group, a piperazinyl group, a pyridinyl group, a pyrrolyl group, a thiazolyl group, an oxazolyl group, a benzoxazolyl group, an indolyl group, a benzothiazolyl group, a benzoimidazolyl group, a benzotriazolyl group, a morphonyl group, a piperidinyl group, or a pyrrolidinyl group is preferable, and a piperidinyl group or a morphonyl group is more preferable.

Among these, from the viewpoint of the adhesiveness and storage stability, A¹ of the above-described diketopyrrolopyrrole compound A is preferably a monovalent organic group having a basic functional group.

In addition, from the viewpoint of the adhesiveness and storage stability, A¹ and B¹ of the above-described diketopyrrolopyrrole compound B are preferably monovalent organic groups having a basic functional group.

In addition, from the viewpoint of the adhesiveness and storage stability, R's in Formula 1 are each independently preferably a hydrogen atom, an alkyl group, or an aryl group, more preferably a hydrogen atom, an alkyl group having 1 to 8 carbon atoms, or an aryl group having 6 to 10 carbon atoms, still more preferably a hydrogen atom or an alkyl group having 1 to 8 carbon atoms, and particularly preferably a hydrogen atom.

In addition, from the viewpoint of the adhesiveness and storage stability, B¹ of the above-described diketopyrrolopyrrole compound A is preferably an aryl group which may have a substituent, and more preferably a phenyl group which may have a substituent.

As the substituent which may be included in the aryl group and phenyl group in B¹ of the above-described diketopyrrolopyrrole compound A, from the viewpoint of the adhesiveness and storage stability, an alkyl group, an alkoxy group, a phthalimidealkyl group, an acyl group, a halogen atom, a phenyl group, a naphthyl group, a cyano group, a trifluoromethyl group, an alkoxycarbonyl group, an alkylthio group, —CONH₂, —CON(R¹¹)R¹², —COOR¹³, —SONR¹⁴R¹⁵, —NR¹⁶SO₂R¹⁷, or —NR¹⁸COR¹⁹ is preferable, an alkyl group, a phenyl group, a naphthyl group, an alkoxy group, or a halogen atom is more preferable, an alkyl group having 1 to 8 carbon atoms, an alkoxy group having 1 to 8 carbon atoms, or a halogen atom is still more preferable, and a methyl group, a methoxy group, or a chlorine atom is particularly preferable.

R¹¹ to R¹⁹ each independently represent an alkyl group having 1 to 20 carbon atoms or a phenyl group.

In addition, the above-described substituent may further have a substituent.

From the viewpoint of the adhesiveness and storage stability, the number of carbon atoms in A¹ of the above-described diketopyrrolopyrrole compound A is preferably 8 to 80, more preferably 9 to 60, and particularly preferably 9 to 40.

From the viewpoint of the adhesiveness and storage stability, the number of carbon atoms in B¹ of the above-described diketopyrrolopyrrole compound A is preferably 6 to 80, more preferably 6 to 60, and particularly preferably 6 to 40.

In addition, from the viewpoint of the adhesiveness and storage stability, the number of carbon atoms in A¹ and B¹ of the above-described diketopyrrolopyrrole compound B is preferably 8 to 80, more preferably 9 to 60, and particularly preferably 9 to 40.

From the viewpoint of the adhesiveness and storage stability, the above-described diketopyrrolopyrrole compound A preferably includes an asymmetric diketopyrrolopyrrole compound represented by Formula 2.

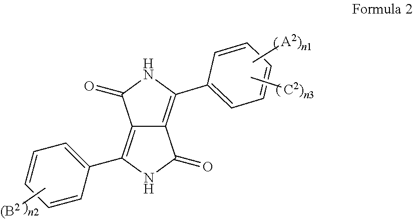

In Formula 2, A²'s each independently represent a monovalent organic group having an acidic functional group or a basic functional group, B²'s each independently represent a monovalent organic group not having an acidic functional group and a basic functional group, C²'s each independently represent a monovalent organic group not having an acidic functional group and a basic functional group, n1 represents an integer of 1 to 5, n2 represents an integer of 0 to 5, n3 represents an integer of 0 to 4, and a phenyl group to which A² and C² are bonded and a phenyl group to which B² is bonded are different groups.

Preferred aspects of the acidic functional group and the basic functional group in A² of Formula 2 are the same as the preferred aspects of the acidic functional group and the basic functional group in A¹ of Formula 1 described above.

From the viewpoint of the adhesiveness and storage stability, A² of Formula 2 is preferably bonded to a benzene ring of Formula 2 through an alkylene group, and more preferably bonded to a benzene ring of Formula 2 through a methylene group.

The number of carbon atoms in A² of Formula 2 is preferably 2 to 60, more preferably 3 to 40, and particularly preferably 9 to 20.

In addition, from the viewpoint of the adhesiveness and storage stability, A² in Formula 2 is preferably —X—Y—Z.

X represents a single bond, —CH₂—, —(CH₂)_{q}O—, —O—, —(CH₂)_{q}S—, —S—, —(CH₂)_{q}COO—, —(CH₂)_{q}SO₂NR¹⁰¹—, —(CH₂)_{q}NR¹⁰¹SO₂—, —(CH₂)_{q}NR¹¹CO—, —(CH₂)_{q}CONR¹⁰¹—, —(CH₂)_{q}NHCOCH₂NH—, —(CH₂)_{q}NHCONH—, —(CH₂)_{q}SO₂—, —(CH₂)_{q}CO—, —(CH₂)_{q}NHCOCH₂—, —(CH₂)_{q}CONHC₆H₄CO—, —(CH₂)_{q}CONHC₆H₄—, or —(CH₂)_{q}NH—,

q represents an integer of 0 to 10,

R¹⁰¹ represents a hydrogen atom, an alkyl group which may have a substituent, an alkenyl group which may have a substituent, or a phenyl group which may have a substituent,

Y represents a single bond, a hydrocarbon group which may have a substitute, an arylene group which may have a substituent, or a heteroaromatic ring which may have a substituent, in which these groups may be linked to each other through a divalent linking group selected from —NR¹⁰¹—, —O—, —SO₂—, and —CO—, and

Z represents a group represented by any of Formulae (Z1) to (Z6).

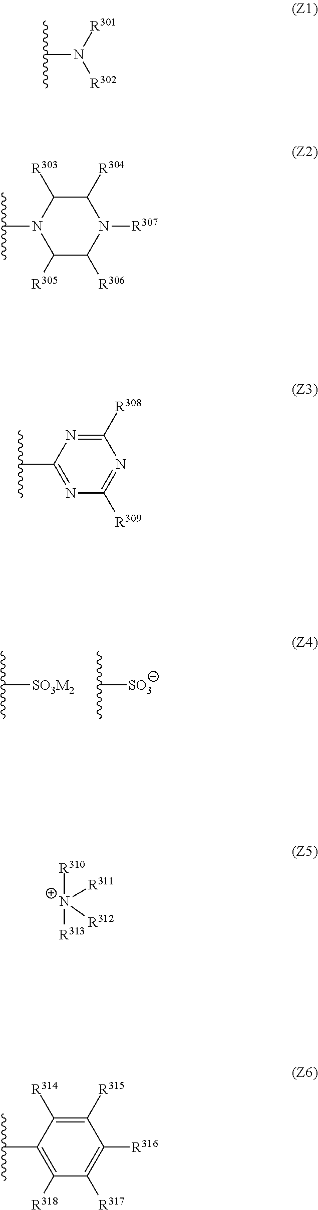

R³⁰¹ and R³⁰² each independently represent a saturated or unsaturated alkyl group which may be substituted or a hetero ring including a nitrogen, oxygen, or sulfur atom, which may be substituted,

R³⁰³, R³⁰⁴, R³⁰⁵, and R³⁰⁶ each independently represent a hydrogen atom, or a saturated or unsaturated alkyl group or aryl group which may be substituted,

R³⁰⁷ represents a saturated or unsaturated alkyl group or aryl group which may be substituted,

R³⁰⁸ and R³⁰⁹ each independently represent a group represented by any of Formula (Z7) or Formula (Z8), —O—(CH₂)ₒ—R³⁵⁰, —OR³⁵¹, —NR³⁵²R³⁵³, —Cl, or —F, in which any one of R³⁰⁸ or R³⁰⁹ is the group represented by any of Formula (Z7) or Formula (Z8), —O—(CH₂)ₒ—R³⁵⁰, —OR³⁵¹, —NR³⁵²R³⁵³, and o represents an integer of 1 to 8,

R³⁵⁰ represents a heterocyclic residue which may have a substituent,

R³⁵¹ to R³⁵³ each independently represent a hydrogen atom, an alkyl group which may have a substituent, an alkenyl group which may have a substituent, or a phenyl group which may have a substituent,

M's each independently represent a metal ion,

R³¹⁰ to R³¹³ each independently represent a hydrogen atom, or a saturated or unsaturated alkyl group or aryl group which may be substituted, and

R³¹⁴ to R³¹⁸ each independently represent a hydrogen atom, an alkoxyl group, an amino group, a sulfo group, a carboxy group, or a phosphorus acidic functional group.

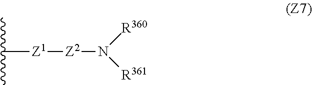

Z¹ represents —NR³⁷⁰—, —CONH—, or —O—,

Z² represents an alkylene group which may have a substituent, an alkenylene group which may have a substituent, or an arylene group which may have a substituent,

these groups may be bonded to each other through a divalent linking group selected from the group consisting of —NR³⁷⁰—, —O—, —SO₂—, and —CO—,

R³⁷⁰ represents a hydrogen atom, an alkyl group which may have a substituent, an alkenyl group which may have a substituent, or a phenyl group which may have a substituent, and

R³⁶⁰ and R³⁶¹ each independently represent an alkyl group which may have a substituent, an alkenyl group which may have a substituent, or a phenyl group which may have a substituent, or a bonding structure of R³⁶⁰ and R³⁶¹ represents a hetero ring including a nitrogen, oxygen, or sulfur atom, which may have a substituent.

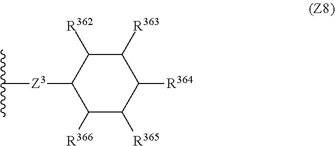

Z³ represents a single bond, —NR³⁸⁰—, —NR³⁸⁰—Z⁴—CO—, —NR³⁸⁰—Z⁴—CONR³⁸¹—, —NR³⁸⁰—Z⁴—SO₂—, —NR³⁸⁰—Z⁴—SO₂NR³⁸¹—, —O—Z⁴—CO—, —O—Z⁴—CONR³⁸⁰—, —O—Z⁴—SO₂—, or —O—Z⁴—SO₂NR³⁸⁰—, which connects the triazine ring and the nitrogen atom,

R³⁶² to R³⁶⁶ each independently represent a hydrogen atom, an alkyl group which may have a substituent, an alkenyl group which may have a substituent, or a phenyl group or polyoxyalkylene group which may have a substituent,

R³⁸⁰ and R³⁸¹ each independently represent a hydrogen atom, an alkyl group which may have a substituent, an alkenyl group which may have a substituent, or a phenyl group which may have a substituent,

Z⁴ represents an alkylene group which may have a substituent, an alkenylene group which may have a substituent, or an arylene group which may have a substituent, and

a wavy line portion represents a bonding position with other structures.

Among these, from the viewpoint of the adhesiveness and storage stability, X is preferably —CH₂—O—, —CH₂—, —O—, —S—, —CH₂—S—, —CH₂—NHSO₂—, or —NHSO₂—.

In addition, from the viewpoint of the adhesiveness and storage stability, Y is preferably a single bond or a substituted or unsubstituted linear or branched hydrocarbon group having 1 to 8 carbon atoms.

Further, from the viewpoint of the adhesiveness and storage stability, Z is preferably the group represented by any of Formulae (Z¹) to (Z⁴), more preferably the group represented by any of Formulae (Z¹) to (Z³), and particularly preferably the group represented by Formula (Z¹).

In addition, from the viewpoint of the adhesiveness and storage stability, A² in Formula 2 is preferably -alkylene group-acidic functional group or basic functional group, -heteroatom-containing linking group-alkylene group-acidic functional group or basic functional group, or -alkylene group-heteroatom-containing linking group-alkylene group-acidic functional group or basic functional group, and more preferably -alkylene group-acidic functional group or basic functional group or -heteroatom-containing linking group-alkylene group-acidic functional group or basic functional group.

From the viewpoint of the adhesiveness and storage stability, the above-described alkylene group is preferably a linear or branched alkylene group having 1 to 8 carbon atoms, and more preferably a linear alkylene group having 1 to 3 carbon atoms.

As the above-described heteroatom-containing linking group, from the viewpoint of the adhesiveness and storage stability, an ether bond, a thioether bond, a sulfonamide bond, or a urea bond is preferable, an ether bond, a sulfonamide bond, or a urea bond is more preferable, an ether bond or a sulfonamide bond is still more preferable, and an ether bond is particularly preferable.

From the viewpoint of the adhesiveness and storage stability, B² and C² in Formula 2 are each independently preferably an alkyl group, an alkoxy group, a phthalimidealkyl group, an acyl group, a halogen atom, a phenyl group, a naphthyl group, a cyano group, a trifluoromethyl group, an alkoxycarbonyl group, an alkylthio group, —CONH₂, —CON(R¹¹)R¹², —COOR¹³, —SONR¹⁴R¹⁵, —NR⁶SO₂R¹⁷, or —NR¹⁸COR¹⁹, more preferably an alkyl group, a phenyl group, a naphthyl group, an alkoxy group, or a halogen atom, still more preferably an alkyl group having 1 to 8 carbon atoms, an alkoxy group having 1 to 8 carbon atoms, a phenyl group, or a halogen atom, and particularly preferably a methyl group, a methoxy group, a phenyl group, a chlorine atom, or a bromine atom.

R¹¹ to R¹⁹ each independently represent an alkyl group having 1 to 20 carbon atoms or a phenyl group.

In addition, the alkyl group, alkoxy group, phthalimidealkyl group, acyl group, halogen atom, phenyl group, naphthyl group, cyano group, trifluoromethyl group, alkoxycarbonyl group, and alkylthio group in B² and C² described above may have a substituent.

Examples of the substituent include an alkyl group, an alkoxy group, a phthalimidealkyl group, an acyl group, a halogen atom, a phenyl group, a naphthyl group, a cyano group, a trifluoromethyl group, an alkoxycarbonyl group, an alkylthio group, —CONH₂, —CON(R¹¹)R¹², —COOR¹³, —SONR¹⁴R¹⁵, —NR¹⁶SO₂R¹⁷, and —NR¹⁸COR¹⁹.

In addition, the number of carbon atoms in B² and C² of Formula 2 is preferably 0 to 60, more preferably 0 to 20, and particularly preferably 0 to 8, respectively.

From the viewpoint of the adhesiveness and storage stability, n1 of Formula 2 is preferably 1 or 2 and more preferably 1.

From the viewpoint of the adhesiveness and storage stability, n2 of Formula 2 is preferably an integer of 0 to 2 and more preferably 0 or 1.

From the viewpoint of the adhesiveness and storage stability, n3 of Formula 2 is preferably 0 or 1 and more preferably 0.

In addition, a bonding position of A², B², and C² in Formula 2 is not particularly limited, but from the viewpoint of the adhesiveness and storage stability, it is preferable to have at least A² at the para position with respect to the bonding position of the diketopyrrolopyrrole ring in the benzene ring of Formula 2.

Further, in a case where n2 is 1 or more, from the viewpoint of the adhesiveness and storage stability, it is preferable to have at least B² at the para position with respect to the bonding position of the diketopyrrolopyrrole ring in the benzene ring of Formula 2.

In addition, from the viewpoint of the adhesiveness and storage stability, the above-described diketopyrrolopyrrole compound A more preferably includes an asymmetric diketopyrrolopyrrole compound represented by Formula 3.

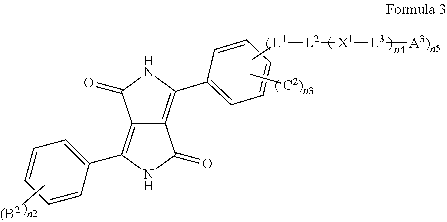

In Formula 3, A 3's each independently represent an acidic functional group or a basic functional group, B²'s each independently represent a monovalent organic group not having an acidic functional group and a basic functional group, C²'s each independently represent a monovalent organic group not having an acidic functional group and a basic functional group, X¹'s each independently represent an ether bond, a thioether bond, a sulfonamide bond, or a urea bond, L¹'s each independently represent a single bond or an ether bond, L²'s and L³'s each independently represent an alkylene group, n2 represents an integer of 0 to 5, n3 represents an integer of 0 to 4, n4's each independently represent 0 or 1, n5 represents an integer of 1 to 5, a group having A³ at a terminal, a phenyl group to which C² is bonded, and a phenyl group to which B² is bonded are different groups, and in a case where L¹ is an ether bond, B² is an electron donating group not having an acidic functional group and a basic functional group and n2 represents an integer of 1 to 5.

Preferred aspects of the acidic functional group and the basic functional group in A³ of Formula 3 are the same as the preferred aspects of the acidic functional group and the basic functional group in A¹ of Formula 1 described above.

Except for the following description, B², C², n2, and n3 of Formula 3 are the same as B², C², n2, and n3 of Formula 2 described above, respectively, and the preferred aspects are also the same.

From the viewpoint of storage stability, L¹ in Formula 3 is preferably a single bond, and from the viewpoint of the adhesiveness, L¹ in Formula 3 is preferably an ether bond.

In addition, in a case where L¹ is an ether bond, from the viewpoint of the adhesiveness, the electron donating group in B² is preferably an alkyl group or an alkoxy group, more preferably an alkyl group having 1 to 8 carbon atoms or an alkoxy group having 1 to 8 carbon atoms, and particularly preferably a methyl group or a methoxy group.

Further, in a case where L¹ is an ether bond, from the viewpoint of the adhesiveness, n2 is preferably an integer of 1 to 3, more preferably 1 or 2, and particularly preferably 1.

From the viewpoint of the adhesiveness and storage stability, L²'s in Formula 3 are each independently preferably a linear or branched alkylene group having 1 to 8 carbon atoms, more preferably a linear alkylene group having 1 to 3 carbon atoms, and particularly preferably a methylene group.

From the viewpoint of the adhesiveness and storage stability, X¹ in Formula 3 is preferably an ether bond, a sulfonamide bond, or a urea bond, more preferably an ether bond or a sulfonamide bond, and particularly preferably an ether bond.

From the viewpoint of the adhesiveness and storage stability, L³'s in Formula 3 are each independently preferably a linear or branched alkylene group having 1 to 8 carbon atoms, more preferably a linear or branched alkylene group having 2 or 3 carbon atoms, and particularly preferably a linear alkylene group having 2 or 3 carbon atoms.

From the viewpoint of the adhesiveness and storage stability, n4 of Formula 3 is preferably 0.

From the viewpoint of the adhesiveness and storage stability, n5 of Formula 3 is preferably 1 or 2 and more preferably 1.

From the viewpoint of the adhesiveness and storage stability, a molecular weight of the above-described diketopyrrolopyrrole compound A is preferably 1,200 or less, more preferably 800 or less, still more preferably 600 or less, and particularly preferably 340 to 600.

The photosensitive coloring composition according to the embodiment of the present disclosure may include only one kind of the above-described diketopyrrolopyrrole compound A, or may include two or more kinds thereof.

The photosensitive coloring composition according to the embodiment of the present disclosure may not include the above-described diketopyrrolopyrrole compound B, may include only one kind thereof, or may include two or more kinds thereof.

From the viewpoint of the adhesiveness and storage stability, a content of the above-described diketopyrrolopyrrole compound A in the photosensitive coloring composition according to the embodiment of the present disclosure is preferably 0.01% by mass to 40% by mass, more preferably 0.05% by mass to 30% by mass, and particularly preferably 0.1% by mass to 20% by mass with respect to the total solid content of the photosensitive coloring composition.

In addition, from the viewpoint of the adhesiveness and storage stability, the total content of the above-described diketopyrrolopyrrole compounds A and B in the photosensitive coloring composition according to the embodiment of the present disclosure is preferably 0.01% by mass to 40% by mass, more preferably 0.05% by mass to 30% by mass, and particularly preferably 0.1% by mass to 20% by mass with respect to the total solid content of the photosensitive coloring composition.

From the viewpoint of the adhesiveness and storage stability, a mass ratio of a content M^{P} of the above-described pigment and a content M^{A} of the above-described diketopyrrolopyrrole compound A in the above-described photosensitive coloring composition is preferably M^{P}/M^{A}=95/5 to 50/50, more preferably 94.9/5.1 to 55/45, still more preferably 94.5/5.5 to 64/36, and particularly preferably 94/6 to 82/18.

Hereinafter, DPP-1 to DPP-26, which are specific examples of the above-described diketopyrrolopyrrole compound A, are shown, but the present disclosure is not limited thereto.

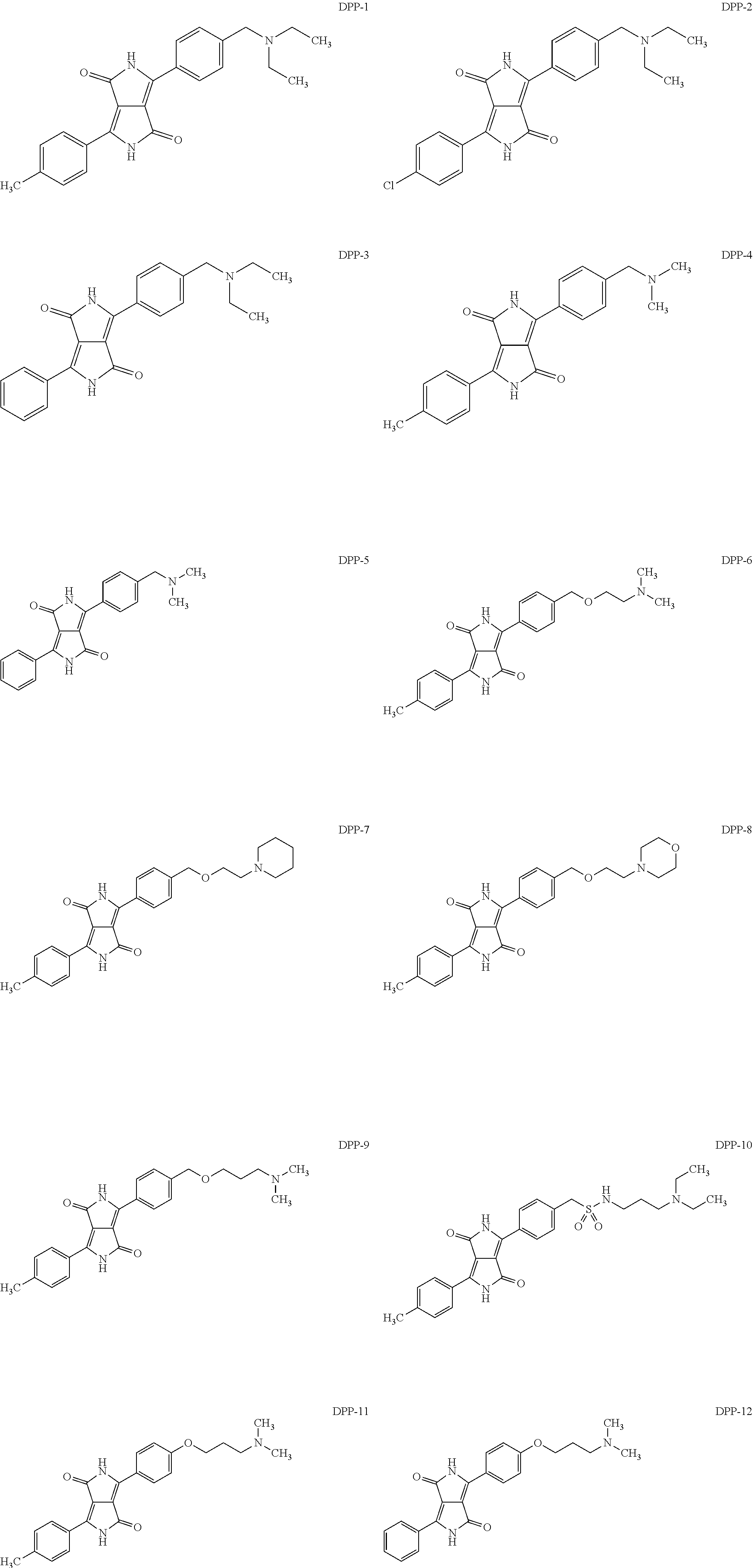

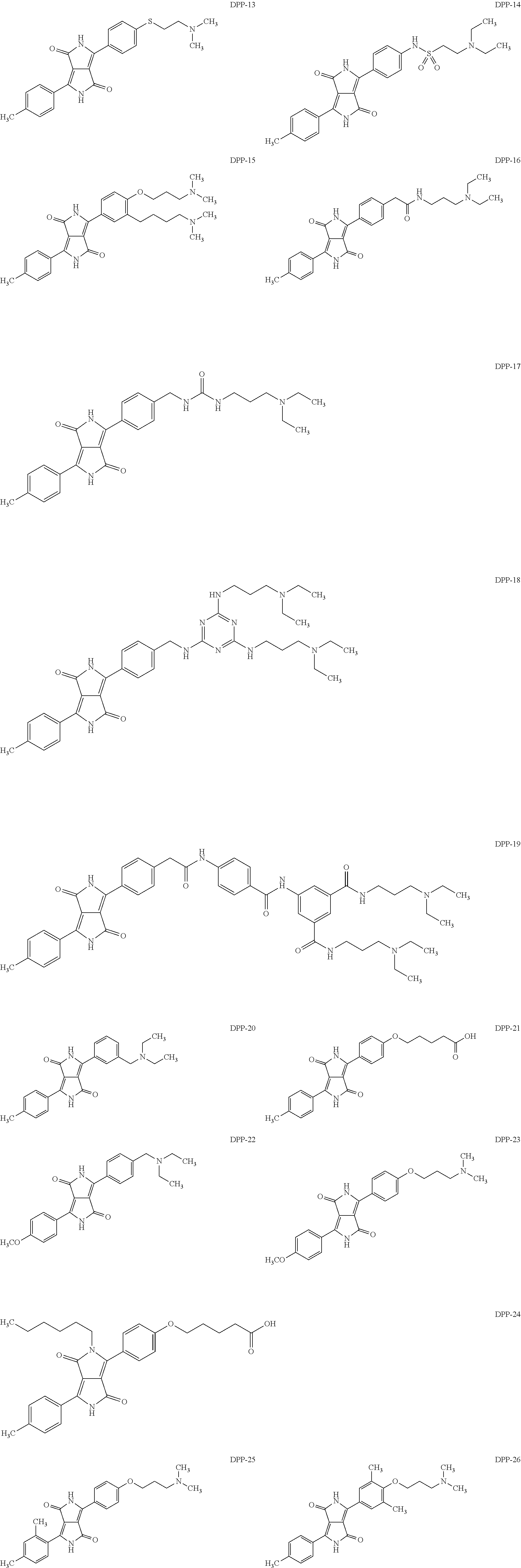

—Method for Producing Diketopyrrolopyrrole Compound A—

A method for producing the diketopyrrolopyrrole compound A is not particularly limited, and the diketopyrrolopyrrole compound A can be produced by referring to a known method. Examples thereof include a method of deriving from a pigment, a method of condensing two or more kinds of cyano compounds, and a method of condensing a cyano compound and a ketopyrrole compound.

Among these, an asymmetric diketopyrrolopyrrole compound can be produced by condensing a cyano compound and a 3-alkoxycarbonyl-5-ketopyrrole compound in the presence of a base.

—Method of Adding Diketopyrrolopyrrole Compounds A and B—

A method of adding the diketopyrrolopyrrole compounds A and B to the photosensitive coloring composition is not particularly limited, and a known addition method and a known mixing method can be used. Preferred examples thereof include a method in which the diketopyrrolopyrrole compound A is mixed (dry or wet) with a pigment in advance and used as a dried diketopyrrolopyrrole compound A-containing composite pigment, a method of adding the diketopyrrolopyrrole compound A during pigment milling, a method of adding the diketopyrrolopyrrole compound A with a dispersion medium during pigment dispersion, and a method of adding the diketopyrrolopyrrole compound A to a pigment dispersion liquid.

<Pigment>

The photosensitive coloring composition according to the embodiment of the present disclosure includes a pigment.

The above-described pigment in the present disclosure is a pigment other than the diketopyrrolopyrrole compounds A and B represented by Formula 1 described above (also referred to as a “compound represented by Formula 1”).

The pigment may be either an inorganic pigment or an organic pigment, but is preferably an organic pigment. In addition, as the pigment, a material in which a part of an inorganic pigment or an organic-inorganic pigment is substituted with an organic chromophore can also be used. By substituting an inorganic pigment or an organic-inorganic pigment with an organic chromophore, hue design can be easily performed.

The photosensitive coloring composition according to the embodiment of the present disclosure can be preferably used as a photosensitive coloring composition for forming a colored pixel in a color filter. Examples of the colored pixel include a red pixel, a green pixel, a blue pixel, a magenta pixel, a cyan pixel, and a yellow pixel. Among these, a red pixel is preferably mentioned.

An average primary particle diameter of the pigment is preferably 1 nm to 200 nm. The lower limit is preferably 5 nm or more and more preferably 10 nm or more. The upper limit is preferably 180 nm or less, more preferably 150 nm or less, and still more preferably 100 nm or less. In a case where the average primary particle diameter of the pigment is within the above-described range, dispersion stability of the pigment in the photosensitive coloring composition is good. In the present disclosure, the primary particle diameter of the pigment can be determined from a captured image obtained by observing primary particles of the pigment using a transmission electron microscope. Specifically, a projected area of the primary particles of the pigment is determined, and the corresponding equivalent circle diameter is calculated as the primary particle diameter of the pigment. In addition, the average primary particle diameter in the present disclosure is an arithmetic average of the primary particle diameters with respect to 400 primary particles of the pigment. In addition, the primary particle of the pigment refers to a particle which is independent without aggregation.

An amount of the pigment dissolved in 100 g of propylene glycol methyl ether acetate at 25° C. is preferably less than 0.01 g, more preferably less than 0.005 g, and still more preferably less than 0.001 g.

Examples of the organic pigment include a phthalocyanine pigment, a dioxazine pigment, a quinacridone pigment, an anthraquinone pigment, a perylene pigment, an azo pigment, a diketopyrrolopyrrole pigment, a pyrolopyrrole pigment, an isoindoline pigment, a quinophthalone pigment, a triarylmethane pigment, a xanthene pigment, a methine pigment, and a quinoline pigment.

Among these, as the pigment, from the viewpoint of the adhesiveness and storage stability, it is preferable to include a diketopyrrolopyrrole pigment other than the compound represented by Formula 1, more preferable to include a diketopyrrolopyrrole red pigment other than the compound represented by Formula 1, still more preferable to include a diaryldiketopyrrolopyrrole red pigment other than the compound represented by Formula 1, and particularly preferable to include a diaryldiketopyrrolopyrrole red pigment other than the compound represented by Formula 1, which has an electron donating group on an aromatic ring.

Specific examples of the organic pigment include the following pigments:

Color Index (C. I.) Pigment Yellow 1, 2, 3, 4, 5, 6, 10, 11, 12, 13, 14, 15, 16, 17, 18, 20, 24, 31, 32, 34, 35, 35:1, 36, 36:1, 37, 37:1, 40, 42, 43, 53, 55, 60, 61, 62, 63, 65, 73, 74, 77, 81, 83, 86, 93, 94, 95, 97, 98, 100, 101, 104, 106, 108, 109, 110, 113, 114, 115, 116, 117, 118, 119, 120, 123, 125, 126, 127, 128, 129, 137, 138, 139, 147, 148, 150, 151, 152, 153, 154, 155, 156, 161, 162, 164, 166, 167, 168, 169, 170, 171, 172, 173, 174, 175, 176, 177, 179, 180, 181, 182, 185, 187, 188, 193, 194, 199, 213, 214, 215, 228, 231, 232 (methine-based), 233 (quinoline-based), 234 (aminoketone-based), 235 (aminoketone-based), 236 (aminoketone-based), and the like (all of which are yellow pigments);

C. I. Pigment Orange 2, 5, 13, 16, 17:1, 31, 34, 36, 38, 43, 46, 48, 49, 51, 52, 55, 59, 60, 61, 62, 64, 71, and 73 (all of which are orange pigments);

C. I. Pigment Red 1, 2, 3, 4, 5, 6, 7, 9, 10, 14, 17, 22, 23, 31, 38, 41, 48:1, 48:2, 48:3, 48:4, 49, 49:1, 49:2, 52:1, 52:2, 53:1, 57:1, 60:1, 63:1, 66, 67, 81:1, 81:2, 81:3, 83, 88, 90, 105, 112, 119, 122, 123, 144, 146, 149, 150, 155, 166, 168, 169, 170, 171, 172, 175, 176, 177, 178, 179, 184, 185, 187, 188, 190, 200, 202, 206, 207, 208, 209, 210, 216, 220, 224, 226, 242, 246, 254, 255, 264, 270, 272, 279, 294 (xanthene-based, Organo Ultramarine, Bluish Red), 295 (monoazo-based), 296 (diazo-based), 297 (aminoketone-based), and the like (all of which are red pigments);

C. I. Pigment Green 7, 10, 36, 37, 58, 59, 62, 63, 64 (phthalocyanine-based), 65 (phthalocyanine-based), 66 (phthalocyanine-based), and the like (all of which are green pigments);

C. I. Pigment Violet 1, 19, 23, 27, 32, 37, 42, 60 (triarylmethane-based), 61 (xanthene-based), and the like (all of which are violet pigments); and

C. I. Pigment Blue 1, 2, 15, 15:1, 15:2, 15:3, 15:4, 15:6, 16, 22, 29, 60, 64, 66, 79, 80, 87 (monoazo-based), 88 (methine-based), and the like (all of which are blue pigments).

As the red pigment, diketopyrrolopyrrole compounds described in JP2017-201384A, in which the structure has at least one substituted bromine atom, diketopyrrolopyrrole compounds described in paragraphs 0016 to 0022 of JP6248838B, diketopyrrolopyrrole compounds described in WO2012/102399A, diketopyrrolopyrrole compounds described in WO2012/117965A, naphtholazo compounds described in JP2012-229344, red pigments described in JP6516119B, red pigments described in JP6525101B, and the like can also be used. In addition, as the red pigment, a compound having a structure that an aromatic ring group in which a group bonded with an oxygen atom, a sulfur atom, or a nitrogen atom is introduced to an aromatic ring is bonded to a diketopyrrolopyrrole skeleton can also be used.

In addition, from the viewpoint of tint and light resistance, preferred examples of the red pigment include C. I. Pigment Red 254, C. I. Pigment Red 255, C. I. Pigment Red 264, and C. I. Pigment Red 272, and more preferred examples thereof include C. I. Pigment Red 254 and C. I. Pigment Red 272.

Further, as the pigment, from the viewpoint of tint, it is preferable to use C. I. Pigment Red 254 and C. I. Pigment Red 272 in combination.

In addition, from the viewpoint of tint, a content mass ratio of C. I. Pigment Red 254 and C. I. Pigment Red 272 is preferably content of C. I. Pigment Red 254:content of C. I. Pigment Red 272=2:1 to 1:2, more preferably 1.5:1 to 1:1.5, still more preferably 1.2:1 to 1:1.2, and particularly preferably 1.2:1 to 1:1.

In addition, from the viewpoint of tint, the pigment preferably includes the red pigment and the yellow pigment.

Further, from the viewpoint of tint, a content mass ratio of the red pigment and the yellow pigment is preferably content of the red pigment:content of the yellow pigment=1:1 to 5:1, and more preferably 1.5:1 to 3:1.

In addition, as the yellow pigment, compounds described in JP2017-201003A, compounds described in JP2017-197719A, compounds described in paragraphs 0011 to 0062 and 0137 to 0276 of JP2017-171912A, compounds described in paragraphs 0010 to 0062 and 0138 to 0295 of JP2017-171913A, compounds described in paragraphs 0011 to 0062 and 0139 to 0190 of JP2017-171914A, compounds described in paragraphs 0010 to 0065 and 0142 to 0222 of JP2017-171915A, quinophthalone compounds described in paragraphs 0011 to 0034 of JP2013-054339A, quinophthalone compounds described in paragraphs 0013 to 0058 of JP2014-026228A, isoindoline compounds described JP2018-062644A, quinophthalone compounds described in JP2018-203798A, quinophthalone compounds described in JP2018-062578A, quinophthalone compounds described in JP6432076B, quinophthalone compounds described in JP2018-155881A, quinophthalone compounds described in JP2018-111757A, quinophthalone compounds described in JP2018-040835A, quinophthalone compounds described in JP2017-197640A, quinophthalone compounds described in JP2016-145282A, quinophthalone compounds described in JP2014-085565A, quinophthalone compounds described in JP2014-021139A, quinophthalone compounds described in JP2013-209614A, quinophthalone compounds described in JP2013-209435A, quinophthalone compounds described in JP2013-181015A, quinophthalone compounds described in JP2013-061622A, quinophthalone compounds described in JP2013-032486A, quinophthalone compounds described in JP2012-226110A, quinophthalone compounds described in JP2008-074987A, quinophthalone compounds described in JP2008-081565A, quinophthalone compounds described in JP2008-074986A, quinophthalone compounds described in JP2008-074985A, quinophthalone compounds described in JP2008-050420A, quinophthalone compounds described in JP2008-031281A, quinophthalone compounds described in JP1973-032765A (JP-S48-032765A), quinophthalone compounds described in JP2019-008014A, quinophthalone compounds described in JP6607427B, methine dyes described in JP2019-073695A, methine dyes described in JP2019-073696A, methine dyes described in JP2019-073697A, methine dyes described in JP2019-073698A, compounds described in KR10-2014-0034963A, compounds described in JP2017-095706A, compounds described in TW2019-20495A, compounds described in JP6607427B, and the like can also be used. In addition, from the viewpoint of improving a color value, a multimerized compound of these compounds is also preferably used.

In addition, from the viewpoint of tint and light resistance, preferred examples of the yellow pigment include C. I. Pigment Yellow 139 and C. I. Pigment Yellow 185.

In addition, a halogenated zinc phthalocyanine pigment having an average number of halogen atoms in one molecule of 10 to 14, an average number of bromine atoms in one molecule of 8 to 12, and an average number of chlorine atoms in one molecule of 2 to 5 can also be used as the green pigment. Specific examples thereof include the compounds described in WO2015/118720A. In addition, as the green pigment, compounds described in CN2010-6909027A, phthalocyanine compounds described in WO2012/102395A, which have phosphoric acid ester as a ligand, phthalocyanine compounds described in JP2019-008014A, phthalocyanine compounds described in JP2018-180023A, compounds described in JP2019-038958A, and the like can also be used.

In addition, an aluminum phthalocyanine compound having a phosphorus atom can also be used as the blue pigment. Specific examples thereof include the compounds described in paragraphs 0022 to 0030 of JP2012-247591A and paragraph 0047 of JP2011-157478A.

In addition, preferred examples of the pigment used in the present disclosure include a pigment having an X-ray diffraction pattern by a specific CuKα ray. Specific examples thereof include phthalocyanine pigments described in JP6561862B, diketopyrrolopyrrole pigments described in JP6413872B, and azo pigments (C. I. Pigment Red 269) described in JP6281345B.

A content of the pigment is preferably 35% by mass or more with respect to the total solid content of the photosensitive coloring composition, and from the viewpoint of the adhesiveness and storage stability, more preferably 40% by mass or more, still more preferably 45% by mass or more, and particularly preferably 50% by mass or more. In addition, the upper limit is preferably 70% by mass or less.

<Resin>

The photosensitive coloring composition according to the embodiment of the present disclosure preferably includes a resin. The resin is blended in, for example, an application for dispersing particles such as a pigment in a photosensitive coloring composition or an application as a binder. Mainly, a resin which is used for dispersing particles such as a pigment is also referred to as a dispersant. However, such applications of the resin are only exemplary, and the resin can also be used for other purposes in addition to such applications.

A weight-average molecular weight (Mw) of the resin is preferably 3,000 to 2,000,000. The upper limit is more preferably 1,000,000 or less and particularly preferably 500,000 or less. The lower limit is more preferably 4,000 or more and particularly preferably 5,000 or more.

Examples of the resin include a (meth)acrylic resin, an ene-thiol resin, a polycarbonate resin, a polyether resin, a polyarylate resin, a polysulfone resin, a polyethersulfone resin, a polyphenylene resin, a polyarylene ether phosphine oxide resin, a polyimide resin, a polyamidoimide resin, a polyolefin resin, a cyclic olefin resin, a polyester resin, and a styrene resin. These resins may be used singly or as a mixture of two or more kinds thereof. In addition, resins described in paragraphs 0041 to 0060 of JP2017-206689A, resins described in paragraphs 0022 to 0071 of JP2018-010856A, resins described in JP2017-057265A, resins described in JP2017-032685A, resins described in JP2017-075248A, and resins described in JP2017-066240A can also be used.

The photosensitive coloring composition according to the embodiment of the present disclosure preferably includes a resin having an acidic functional group as the resin. According to this aspect, developability of the photosensitive coloring composition can be improved, and a pixel having excellent rectangularity can be easily formed. Examples of the acidic functional group include a carboxy group, a phosphoric acidic functional group, a sulfo group, and a phenolic hydroxy group, and a carboxy group is preferable. The resin having an acidic functional group can be used, for example, as an alkali-soluble resin.

In addition, from the viewpoint of the adhesiveness and storage stability, the photosensitive coloring composition according to the embodiment of the present disclosure preferably includes the diketopyrrolopyrrole compound represented by Formula 1 described above, in which A¹ is a monovalent organic group having a basic functional group, and a resin having an acidic functional group.

The resin having an acidic functional group preferably includes a repeating unit having an acidic functional group in the side chain, and more preferably includes 5 mol % to 70 mol % of repeating units having an acidic functional group in the side chain with respect to the total repeating units of the resin. The upper limit of the content of the repeating unit having an acidic functional group in the side chain is preferably 50 mol % or less and more preferably 30 mol % or less. The lower limit of the content of the repeating unit having an acidic functional group in the side chain is preferably 10 mol % or more and more preferably 20 mol % or more.

It is also preferable that the resin having an acidic functional group includes a repeating unit derived from a monomer component including at least one monomer selected from the group consisting of a compound represented by Formula (ED1) and a compound represented by Formula (ED2) (hereinafter, these compounds may be referred to as an “ether dimer”).

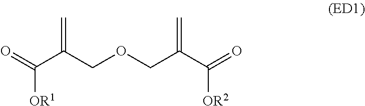

In Formula (ED1), R¹ and R² each independently represent a hydrogen atom or a hydrocarbon group having 1 to 25 carbon atoms, which may have a substituent.

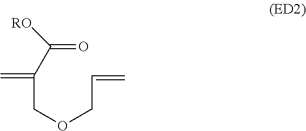

In Formula (ED2), R represents a hydrogen atom or an organic group having 1 to 30 carbon atoms. With regard to details of Formula (ED2), reference can be made to the description in JP2010-168539A, the contents of which are incorporated herein by reference.

With regard to the specific examples of the ether dimer, reference can be made to the description in paragraph 0317 of JP2013-029760A, the contents of which are incorporated herein by reference.

It is also preferable that the resin used in the present disclosure includes a repeating unit derived from a compound represented by Formula (X).

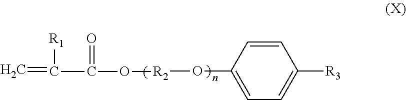

In Formula (X), R₁ represents a hydrogen atom or a methyl group, R₂ represents an alkylene group having 2 to 10 carbon atoms, and R₃ represents a hydrogen atom or an alkyl group having 1 to 20 carbon atoms which may include a benzene ring. n represents an integer of 1 to 15.

With regard to the resin having an acidic functional group, reference can be made to the description in paragraphs 0558 to 0571 of JP2012-208494A (paragraphs 0685 to 0700 of the corresponding US2012/0235099A) and the description in paragraphs 0076 to 0099 of JP2012-198408A, the contents of which are incorporated herein by reference. A commercially available product can also be used as the resin having an acidic functional group.

An acid value of the resin having an acidic functional group is preferably 30 mgKOH/g to 500 mgKOH/g. The lower limit is preferably 40 mgKOH/g or more and more preferably 50 mgKOH/g or more. The upper limit is more preferably 400 mgKOH/g or less, still more preferably 300 mgKOH/g or less, and particularly preferably 200 mgKOH/g or less. A weight-average molecular weight (Mw) of the resin having an acidic functional group is preferably 5,000 to 100,000. In addition, a number-average molecular weight (Mn) of the resin having an acidic functional group is preferably 1,000 to 30,000.

In addition, a method of introducing the acidic functional group into the resin is not particularly limited, and examples thereof include the method described in JP6349629B.

Further, examples of the method of introducing the acidic functional group into the resin include a method of introducing an acid group by reacting an acid anhydride with a hydroxy group generated by a ring-opening reaction of an epoxy group in a dispersant (particularly, a dispersant having an ethylenically unsaturated group, and the like) or an alkali-soluble resin.

In the present disclosure, as the resin, a resin having a basic functional group can be preferably used. According to this aspect, developability of the photosensitive coloring composition can be improved, and a pixel having excellent rectangularity can be easily formed. Examples of the basic functional group include an amino group and a heteroaryl group having a nitrogen atom, and an amino group is preferable and a tertiary amino group is more preferable. The resin having a basic functional group can be used, for example, as an alkali-soluble resin.

An amine value of the resin having an amino group as the basic functional group is preferably 20 mgKOH/g to 200 mgKOH/g. The lower limit is preferably 30 mgKOH/g or more and more preferably 40 mgKOH/g or more. The upper limit is preferably 180 mgKOH/g or less, more preferably 160 mgKOH/g or less, and still more preferably 140 mgKOH/g or less. A weight-average molecular weight (Mw) of the resin having an amino group is preferably 5,000 to 100,000. In addition, a number-average molecular weight (Mn) of the resin having an amino group is preferably 1,000 to 20,000.

The photosensitive coloring composition according to the embodiment of the present disclosure can also include a resin as a dispersant. Examples of the dispersant include an acidic dispersant (acidic resin) and a basic dispersant (basic resin). Here, the acidic dispersant (acidic resin) represents a resin in which the amount of the acidic functional group is larger than the amount of the basic functional group. The acidic dispersant (acidic resin) is preferably a resin in which the amount of the acidic functional group occupies 70 mol % or more in a case where the total amount of the acidic functional group and the basic functional group is 100 mol %, and more preferably a resin substantially consisting of only an acidic functional group. The acidic functional group included in the acidic dispersant (acidic resin) is preferably a carboxy group. An acid value of the acidic dispersant (acidic resin) is preferably 30 mgKOH/g to 105 mgKOH/g, more preferably 40 mgKOH/g to 105 mgKOH/g, and still more preferably 50 mgKOH/g to 105 mgKOH/g. In addition, the basic dispersant (basic resin) represents a resin in which the amount of the basic functional group is larger than the amount of the acidic functional group. The basic dispersant (basic resin) is preferably a resin in which the amount of the basic functional group is more than 50 mol % in a case where the total amount of the acidic functional group and the basic functional group is 100 mol %. The basic functional group included in the basic dispersant is preferably an amino group.

The resin used as a dispersant preferably includes a repeating unit having an acidic functional group. In a case where the resin used as a dispersant includes a repeating unit having an acidic functional group, the generation of the development residue can be further suppressed in the formation of a pattern by a photolithography method.

It is also preferable that the resin used as a dispersant is a graft resin. With regard to details of the graft resin, reference can be made to the description in paragraphs 0025 to 0094 of JP2012-255128A, the contents of which are incorporated herein by reference.

It is also preferable that the resin used as a dispersant is a polyimine-based dispersant including a nitrogen atom in at least one of the main chain or the side chain. As the polyimine-based dispersant, a resin having a main chain which has a partial structure having a functional group of pKa 14 or less, and a side chain which has 40 to 10,000 atoms, in which at least one of the main chain or the side chain has a basic nitrogen atom, is preferable. The basic nitrogen atom is not particularly limited as long as it is a nitrogen atom exhibiting basicity. With regard to the polyimine-based dispersant, reference can be made to the description in paragraphs 0102 to 0166 of JP2012-255128A, the contents of which are incorporated herein by reference.

It is also preferable that the resin used as a dispersant is a resin having a structure in which a plurality of polymer chains are bonded to a core portion. Examples of such a resin include dendrimers (including star polymers). In addition, specific examples of the dendrimer include polymer compounds C-1 to C-31 described in paragraphs 0196 to 0209 of JP2013-043962A.

In addition, the above-described resin (alkali-soluble resin) having an acidic functional group can also be used as a dispersant.

In addition, it is also preferable that the resin used as a dispersant is a resin including a repeating unit having an ethylenically unsaturated bond-containing group in the side chain. A content of the repeating unit having an ethylenically unsaturated bond-containing group in the side chain is preferably 10 mol % or more, more preferably 10 mol % to 80 mol %, and still more preferably 20 mol % to 70 mol % with respect to the total repeating units of the resin.

In addition, preferred examples of the dispersant include a resin having an aromatic carboxy group (hereinafter, a “resin B”).

The resin B may include the aromatic carboxy group in the main chain of the repeating unit, or in the side chain of the repeating unit. From the reason that it is excellent in developability and color loss, it is preferable that the aromatic carboxy group is included in the main chain of the repeating unit. The details are not clear, but it is presumed that the presence of the aromatic carboxy group near the main chain further improves these properties. In the present specification, the aromatic carboxyl group is a group having a structure in which one or more carboxyl groups are bonded to an aromatic ring. In the aromatic carboxy group, the number of carboxy groups bonded to an aromatic ring is preferably 1 to 4 and more preferably 1 or 2.

The resin B used in the present disclosure is preferably a resin including at least one repeating unit selected from a repeating unit represented by Formula (b-1) or a repeating unit represented by Formula (b-10).

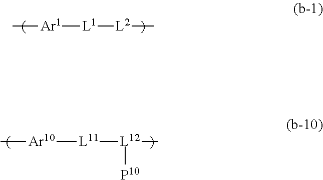

In Formula (b-1), Ar¹ represents a group including an aromatic carboxyl group, L¹ represents —COO— or —CONH—, and L² represents a divalent linking group.

In Formula (b-10), Ar¹⁰ represents a group including an aromatic carboxyl group, L¹ represents —COO— or —CONH—, L¹² represents a trivalent linking group, and P¹⁰ represents a polymer chain.

First, Formula (b-1) will be described. In Formula (b-1), examples of the group including an aromatic carboxy group, represented by Ar¹, include a structure derived from an aromatic tricarboxylic acid anhydride and a structure derived from an aromatic tetracarboxylic acid anhydride. Examples of the aromatic tricarboxylic acid anhydride and the aromatic tetracarboxylic acid anhydride include compounds having the following structures.

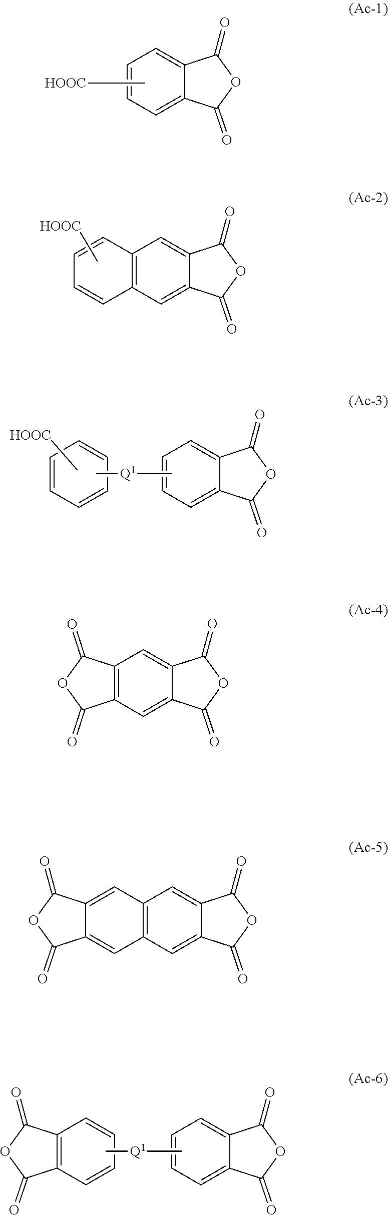

In the formulae, Q¹ represents a single bond, —O—, —CO—, —COOCH₂CH₂OCO—, —SO₂—, —C(CF₃)₂—, a group represented by Formula (Q-1), or a group represented by Formula (Q-2).

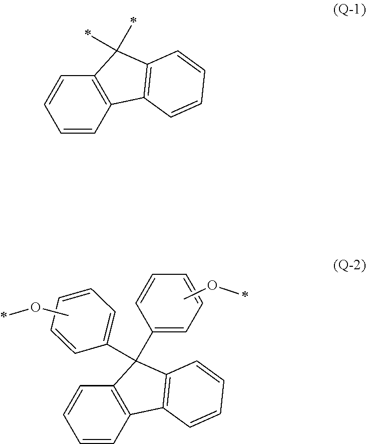

Specific examples of the aromatic tricarboxylic acid anhydride include a benzenetricarboxylic acid anhydride (1,2,3-benzenetricarboxylic acid anhydride, trimellitic acid anhydride [1,2,4-benzenetricarboxylic acid anhydride], and the like), a naphthalenetricarboxylic acid anhydride (1,2,4-naphthalenetricarboxylic acid anhydride, 1,4,5-naphthalenetricarboxylic acid anhydride, 2,3,6-naphthalenetricarboxylic acid anhydride, 1,2,8-naphthalenetricarboxylic acid anhydride, and the like), 3,4,4′-benzophenonetricarboxylic acid anhydride, 3,4,4′-biphenylethertricarboxylic acid anhydride, 3,4,4′-biphenyltricarboxylic acid anhydride, 2,3,2′-biphenyltricarboxylic acid anhydride, 3,4,4′-biphenylmethanetricarboxylic acid anhydride, and 3,4,4′-biphenylsulfonetricarboxylic acid anhydride. Specific examples of the aromatic tetracarboxylic acid anhydride include pyromellitic acid dianhydride, ethylene glycol dianhydrous trimellitic acid ester, propylene glycol dianhydrous trimellitic acid ester, butylene glycol dianhydrous trimellitic acid ester, 3,3′,4,4′-benzophenonetetracarboxylic acid dianhydride, 3,3′,4,4′-biphenylsulfonetetracarboxylic acid dianhydride, 1,4,5,8-naphthalenetetracarboxylic acid dianhydride, 2,3,6,7-naphthalenetetracarboxylic acid dianhydride, 3,3′,4,4′-biphenylethertetracarboxylic acid dianhydride, 3,3′,4,4′-dimethyldiphenylsilanetetracarboxylic acid dianhydride, 3,3′,4,4′-tetraphenylsilanetetracarboxylic acid dianhydride, 1,2,3,4-frantetracarboxylic acid dianhydride, 4,4′-bis(3,4-dicarboxyphenoxy)diphenylsulfide dianhydride, 4,4′-bis(3,4-dicarboxyphenoxy)diphenylsulfone dianhydride, 4,4′-bis (3,4-dicarboxyphenoxy) diphenylpropane dianhydride, 3,3′,4,4′-perfluoroisopropyridendiphthalic acid dianhydride, 3,3′,4,4′-biphenyltetracarboxylic acid dianhydride, bis(phthalic acid) phenylphosphineoxide dianhydride, p-phenylene-bis(triphenylphthalic acid) dianhydride, m-phenylene-bis(triphenylphthalic acid) dianhydride, bis(triphenylphthalic acid)-4,4′-diphenylether dianhydride, bis(triphenylphthalic acid)-4,4′-diphenylmethane dianhydride, 9,9-bis(3,4-dicarboxyphenyl)fluorene dianhydride, 9,9-bis[4-(3,4-dicarboxyphenoxy)phenyl]fluorene dianhydride, 3,4-dicarboxy-1,2,3,4-tetrahydro-1-naphthalenesuccinic acid dianhydride, and 3,4-dicarboxy-1,2,3,4-tetrahydro-6-methyl-1-naphthalenesuccinic acid dianhydride.

Specific examples of the group including an aromatic carboxyl group represented by Ar¹ include a group represented by Formula (Ar-1), a group represented by Formula (Ar-2), and a group represented by Formula (Ar-3).

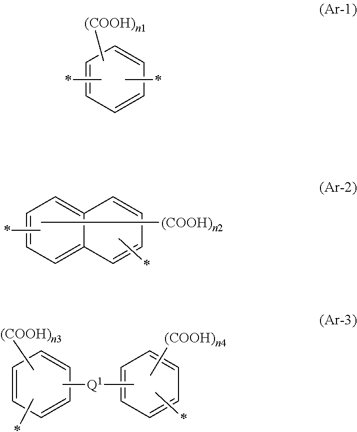

In Formula (Ar-1), n1 represents an integer of 1 to 4, and is preferably an integer of 1 or 2 and more preferably 2.

In Formula (Ar-2), n2 represents an integer of 1 to 8, and is preferably an integer of 1 or 4, more preferably 1 or 2, and still more preferably 2.

In Formula (Ar-3), n3 and n4 each independently represent an integer of 0 to 4, and are preferably an integer of 0 or 2, more preferably 1 or 2, and still more preferably 1. However, at least one of n3 or n4 is an integer of 1 or more.

In Formula (Ac-3), Q¹ represents a single bond, —O—, —CO—, —COOCH₂CH₂OCO—, —SO₂—, —C(CF₃)₂—, the above-described group represented by Formula (Q-1), or the above-described group represented by Formula (Q-2).

In Formula (b-1), L¹ represents —COO— or —CONH—, preferably —COO—.

In Formula (b-1), examples of the divalent linking group represented by L² include an alkylene group, an arylene group, —O—, —CO—, —COO—, —OCO—, —NH—, —S—, and a group formed by a combination of two or more of these groups. The number of carbon atoms in the alkylene group preferably is 1 to 30, more preferably 1 to 20, and still more preferably 1 to 15. The alkylene group may be linear, branched, or cyclic. The number of carbon atoms in the arylene group is preferably 6 to 30, more preferably 6 to 20, and still more preferably 6 to 10. The alkylene group and the arylene group may have a substituent. Examples of the substituent include a hydroxy group. The divalent linking group represented by L² is preferably a group represented by —O-L^{2a}-O—. Examples of L²a include an alkylene group; an arylene group; a group formed by a combination of an alkylene group and an arylene group; and a group formed by a combination of at least one selected from an alkylene group or an arylene group, and at least one selected from the group consisting of —O—, —CO—, —COO—, —OCO—, —NH—, and —S—. The number of carbon atoms in the alkylene group preferably is 1 to 30, more preferably 1 to 20, and still more preferably 1 to 15. The alkylene group may be linear, branched, or cyclic. The alkylene group and the arylene group may have a substituent. Examples of the substituent include a hydroxy group.

Next, Formula (b-10) will be described. In Formula (b-10), the group including an aromatic carboxy group, represented by Ar¹⁰, has the same meaning as Ar¹ in Formula (b-1), and the preferred range is also the same.

In Formula (b-10), L¹¹ represents —COO— or —CONH—, preferably —COO—.

In Formula (b-10), examples of the trivalent linking group represented by L¹² include a hydrocarbon group, —O—, —CO—, —COO—, —OCO—, —NH—, —S—, and a group formed by a combination of two or more of these groups. Examples of the hydrocarbon group include an aliphatic hydrocarbon group and an aromatic hydrocarbon group. The number of carbon atoms in the aliphatic hydrocarbon group is preferably 1 to 30, more preferably 1 to 20, and still more preferably 1 to 15. The aliphatic hydrocarbon group may be linear, branched, or cyclic. The number of carbon atoms in the aromatic hydrocarbon group is preferably 6 to 30, more preferably 6 to 20, and still more preferably 6 to 10. The hydrocarbon group may have a substituent. Examples of the substituent include a hydroxy group. The trivalent linking group represented by L¹² is preferably a group represented by Formula (L12-1), and more preferably a group represented by Formula (L12-2).

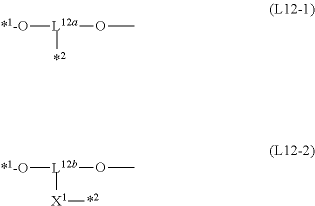

L^{12a} and L^{12b} each independently represent a trivalent linking group, X¹ represents S, *1 represents a bonding position with L¹¹ in Formula (b-10), and *2 represents a bonding position with P¹⁰ in Formula (b-10).

Examples of the trivalent linking group represented by L^{12a} and L^{12b} include a hydrocarbon group; and a group formed by a combination of a hydrocarbon group and at least one selected from the group consisting of —O—, —CO—, —COO—, —OCO—, —NH—, and —S—.

In Formula (b-10), P¹⁰ represents a polymer chain. It is preferable that the polymer chain represented by P¹⁰ has at least one repeating unit selected from a poly(meth)acrylic repeating unit, a polyether repeating unit, a polyester repeating unit, or a polyol repeating unit. A weight-average molecular weight of the polymer chain P¹⁰ is preferably 500 to 20,000. The lower limit is more preferably 500 or more and still more preferably 1,000 or more. The upper limit is more preferably 10,000 or less, still more preferably 5,000 or less, and particularly preferably 3,000 or less. In a case where the weight-average molecular weight of P¹⁰ is within the above-described range, dispersibility of the pigment in the composition is good. In a case where the resin B is a resin having the repeating unit represented by Formula (b-10), the resin B is preferably used as a dispersant.

In Formula (b-10), the polymer chain represented by P¹⁰ is preferably a polymer chain including a repeating unit represented by Formulae (P-1) to (P-5), and more preferably a polymer chain including a repeating unit represented by Formula (P-5).

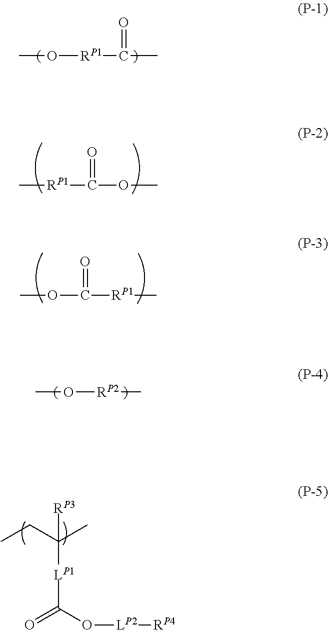

In the formulae, R^{P1} and R^{P2} each represent an alkylene group. As the alkylene group represented by R^{P1} and R^{P2}, a linear or branched alkylene group having 1 to 20 carbon atoms is preferable, a linear or branched alkylene group having 2 to 16 carbon atoms is more preferable, and a linear or branched alkylene group having 3 to 12 carbon atoms is still more preferable.

In the formulae, R^{P3} represents a hydrogen atom or a methyl group.

In the formulae, L^{P1} represents a single bond or an arylene group and L^{P2} represents a single bond or a divalent linking group. L^{P1} is preferably a single bond. Examples of the divalent linking group represented by L^{P2} include an alkylene group (preferably an alkylene group having 1 to 12 carbon atoms), an arylene group (preferably an arylene group having 6 to 20 carbon atoms), —NH—, —SO—, —SO₂—, —CO—, —O—, —COO—, —OCO—, —S—, —NHCO—, —CONH—, and a group formed by a combination of two or more these groups.

R^{N} represents a hydrogen atom or a substituent. Examples of the substituent include a hydroxy group, a carboxy group, an alkyl group, an aryl group, a heteroaryl group, an alkoxy group, an aryloxy group, a heteroaryloxy group, an alkylthioether group, an arylthioether group, a heteroarylthioether group, a (meth)acryloyl group, an oxetanyl group, and a blocked isocyanate group. The blocked isocyanate group in the present disclosure is a group capable of generating an isocyanate group by heat, and preferred examples thereof include a group in which an isocyanate group is protected by reacting a blocking agent and an isocyanate group. Examples of the blocking agent include oxime compounds, lactam compounds, phenol compounds, alcohol compounds, amine compounds, active methylene compounds, pyrazole compounds, mercaptan compounds, imidazole compounds, and imide compounds. Examples of the blocking agent include compounds described in paragraphs 0115 to 0117 of JP2017-067930A, the contents of which are incorporated herein by reference. In addition, the blocked isocyanate group is preferably a group capable of generating an isocyanate group by heat of 90° C. to 260° C.

It is preferable that the polymer chain represented by P¹⁰ has at least one group (hereinafter, also referred to as a “functional group A”) selected from the group consisting of a (meth)acryloyl group, an oxetanyl group, a blocked isocyanate group, and a t-butyl group. The functional group A is more preferably at least one selected from the group consisting of a (meth)acryloyl group, an oxetanyl group, and a blocked isocyanate group. In a case where the polymer chain includes the functional group A, it is easy to form a film having excellent solvent resistance. In particular, the effects described above are remarkable in a case of including at least one group selected from a (meth)acryloyl group, an oxetanyl group, and a blocked isocyanate group. In addition, in a case where the functional group A has a t-butyl group, it is preferable that the composition includes a compound having an epoxy group or an oxetanyl group. In a case where the functional group A has a blocked isocyanate group, it is preferable that the composition includes a compound having a hydroxy group.

In addition, it is more preferable that the polymer chain represented by P¹⁰ is a polymer chain having a repeating unit including the above-described functional group A in the side chain. In addition, the proportion of the repeating unit including the above-described functional group A in the side chain with respect to total repeating units constituting P¹⁰ is preferably 5% by mass or more, more preferably 10% by mass or more, and still more preferably 20% by mass or more. The upper limit may be 100% by mass, and is preferably 90% by mass or less and more preferably 60% by mass or less.

In addition, it is also preferable that the polymer chain represented by P¹⁰ has a repeating unit including an acidic functional group. Examples of the acidic functional group include a carboxy group, a phosphoric acidic functional group, a sulfo group, and a phenolic hydroxy group. According to this aspect, the dispersibility of the pigment in the composition can be further improved. Furthermore, developability can also be further improved. A proportion of the repeating unit including an acidic functional group is preferably 1% by mass to 30% by mass, more preferably 2% by mass to 20% by mass, and still more preferably 3% by mass to 10% by mass.

The resin B can be manufactured by reacting at least one acid anhydride selected from the group consisting of an aromatic tetracarboxylic acid anhydride and an aromatic tricarboxylic acid anhydride with a hydroxy group-containing compound. Examples of the aromatic tetracarboxylic acid anhydride and the aromatic tricarboxylic acid anhydride include those described above. The hydroxy group-containing compound is not particularly limited as long as it has a hydroxy group in the molecule, but is preferably a polyol having two or more hydroxy groups in the molecule. In addition, as the hydroxy group-containing compound, it is also preferable to use a compound having two hydroxy groups and one thiol group in the molecule. Examples of the compound having two hydroxy groups and one thiol group in the molecule include 1-mercapto-1,1-methanediol, 1-mercapto-1,1-ethanediol, 3-mercapto-1,2-propanediol (thioglycerin), 2-mercapto-1,2-propanediol, 2-mercapto-2-methyl-1,3-propanediol, 2-mercapto-2-ethyl-1,3-propanediol, 1-mercapto-2,2-propanediol, 2-mercaptoethyl-2-methyl-1,3-propanediol, and 2-mercaptoethyl-2-ethyl-1,3-propanediol. Examples of other hydroxy group-containing compounds include compounds described in paragraphs 0084 to 0095 of JP2018-101039A, the contents of which are incorporated herein by reference.

A molar ratio (acid anhydride group/hydroxy group) of the acid anhydride group in the acid anhydride to the hydroxy group in the hydroxy group-containing compound is preferably 0.5 to 1.5.

In addition, the above-described resin including the repeating unit represented by Formula (b-10) can be synthesized by the methods shown in the following synthesis methods (1) and (2).

[Synthesis Method (1)]

Producing method of radically polymerizing a polymerizable monomer having an ethylenically unsaturated group in the presence of a hydroxy group-containing thiol compound (preferably a compound having two hydroxy groups and one thiol group in the molecule) to synthesize a vinyl polymer having two hydroxy groups in one terminal region, and reacting the synthesized vinyl polymer with one or more aromatic acid anhydride selected from the group consisting of the aromatic tetracarboxylic acid anhydride and the aromatic tricarboxylic acid anhydride.

[Synthesis Method (2)]

Producing method of reacting a hydroxy group-containing compound (preferably a compound having two hydroxy groups and one thiol group in the molecule) with one or more aromatic acid anhydride selected from the group consisting of the aromatic tetracarboxylic acid anhydride and the aromatic tricarboxylic acid anhydride, and radically polymerizing a polymerizable monomer having an ethylenically unsaturated group in the presence of the obtained reactant. In the synthesis method (2), after radically polymerizing the polymerizable monomer having a hydroxy group, the reactant may be further reacted with a compound having an isocyanate group (for example, a compound having an isocyanate group and the above-described functional group A). As a result, the functional group A can be introduced into the polymer chain P¹⁰

In addition, the resin B can also be synthesized according to the method described in paragraphs 0120 to 0138 of JP2018-101039A.

A weight-average molecular weight of the resin B is preferably 2,000 to 35,000. The upper limit is more preferably 25,000 or less, still more preferably 20,000 or less, and particularly preferably 15,000 or less. The lower limit is more preferably 4,000 or more, still more preferably 6,000 or more, and particularly preferably 7,000 or more. In a case where the weight-average molecular weight of the resin B is within the above-described range, the effects in the present disclosure are more remarkably obtained. In addition, storage stability of the photosensitive coloring composition can also be improved.

A commercially available product is also available as the dispersant, and specific examples thereof include DISPERBYK series (for example, DISPERBYK-111, 161, and the like) manufactured by BYK Chemie, and Solsperse series (for example, Solsperse 76500) manufactured by Lubrizol Corporation. Dispersing agents described in paragraphs 0041 to 0130 of JP2014-130338A can also be used, the contents of which are incorporated herein by reference. The resin described as a dispersant can be used for an application other than the dispersant. For example, the resin can also be used as a binder.

In a case where the photosensitive coloring composition according to the embodiment of the present disclosure contains a resin, a content of the resin in the total solid content of the coloring composition is preferably 5% by mass to 50% by mass. The lower limit is more preferably 10% by mass or more and particularly preferably 15% by mass or more. The upper limit is more preferably 40% by mass or less, still more preferably 35% by mass or less, and particularly preferably 30% by mass or less. In addition, the content of the resin (alkali-soluble resin) having an acidic functional group in the total solid content of the photosensitive coloring composition is preferably 5% by mass to 50% by mass. The lower limit is more preferably 10% by mass or more and particularly preferably 15% by mass or more. The upper limit is more preferably 40% by mass or less, still more preferably 35% by mass or less, and particularly preferably 30% by mass or less. In addition, from the reason that excellent developability is easily obtained, the content of the resin (alkali-soluble resin) having an acidic functional group in the total amount of the resin is preferably 30% by mass or more, more preferably 50% by mass or more, still more preferably 70% by mass or more, and particularly preferably 80% by mass or more. The upper limit may be 100% by mass, 95% by mass, or 90% by mass or less. In the photosensitive coloring composition according to the embodiment of the present disclosure, the resin may be used singly or in a combination of two or more kinds thereof. In a case where two or more kinds thereof are used in combination, the total amount thereof is preferably within the above-described range.

<Pigment Derivative>

The photosensitive coloring composition according to the embodiment of the present disclosure can contain a pigment derivative. The “pigment derivative” in the present disclosure is a pigment derivative other than the diketopyrrolopyrrole compounds A and B represented by Formula 1 described above.

Examples of the pigment derivative include a compound having a structure in which a part of a chromophore is substituted with an acidic functional group or a basic functional group. Examples of the chromophore constituting the pigment derivative include a quinoline skeleton, a benzimidazolone skeleton, a diketopyrrolopyrrole skeleton, an azo skeleton, a phthalocyanine skeleton, an anthraquinone skeleton, a quinacridone skeleton, a dioxazine skeleton, a perinone skeleton, a perylene skeleton, a thioindigo skeleton, an isoindoline skeleton, an isoindolinone skeleton, a quinophthalone skeleton, a threne skeleton, and a metal complex-based skeleton. Among these, a quinoline skeleton, a benzimidazolone skeleton, a diketopyrrolopyrrole skeleton, an azo skeleton, a quinophthalone skeleton, an isoindoline skeleton, or a phthalocyanine skeleton is preferable, and an azo skeleton or a benzimidazolone skeleton is more preferable. Examples of the acidic functional group include a sulfo group, a carboxy group, a phosphoric acidic functional group, and a salt thereof. Examples of an atom or atomic group constituting the salts include alkali metal ions (Li⁺, Na⁺, K⁺, and the like), alkaline earth metal ions (Ca²⁺, Mg²⁺, and the like), an ammonium ion, an imidazolium ion, a pyridinium ion, and a phosphonium ion. Examples of the basic functional group included in the pigment derivative include an amino group, a pyridinyl group, or a salt thereof, a salt of an ammonium group, and a phthalimidomethyl group. Examples of an atom or atomic group constituting the salts include a hydroxide ion, a halogen ion, a carboxylate ion, a sulfonate ion, and a phenoxide ion.

As the pigment derivative, a pigment derivative having excellent visible transparency (hereinafter, also referred to as a transparent pigment derivative) can be used. The maximum value (εmax) of a molar absorption coefficient of the transparent pigment derivative in a wavelength range of 400 nm to 700 nm is preferably 3,000 L·mol⁻¹ cm⁻¹ or less, more preferably 1,000 L·mol⁻¹ cm⁻¹ or less, and still more preferably 100 L·mol⁻¹ cm⁻¹ or less. The lower limit of εmax is, for example, 1 L·mol⁻¹ cm⁻¹ or more and may be 10 L·mol⁻¹ cm⁻¹ or more.

Specific examples of the pigment derivative include compounds described in JP1981-118462A (JP-S56-118462A), JP1988-264674A (JP-S63-264674A), JP1989-217077A (JP-H01-217077A), JP1991-009961A (JP-H03-009961A), JP1991-026767A (JP-H03-026767A), JP1991-153780A (JP-H03-153780A), JP1991-045662A (JP-H03-045662A), JP1992-285669A (JP-H04-285669A), JP1994-145546A (JP-H06-145546A), JP1994-212088A (JP-H06-212088A), JP1994-240158A (JP-H06-240158A), JP1998-030063A (JP-H10-030063A), JP1998-195326A (JP-H10-195326A), paragraphs 0086 to 0098 of WO2011/024896A, paragraphs 0063 to 0094 of WO2012/102399A, paragraph 0082 of WO2017/038252A, paragraph 0171 of JP2015-151530A, paragraphs 0162 to 0183 of JP2011-252065A, JP2003-081972A, JP5299151B, JP2015-172732A, JP2014-199308A, JP2014-085562A, JP2014-035351A, JP2008-081565A, and JP2019-109512A.

A content of the pigment derivative is preferably 1 part by mass to 30 parts by mass, and more preferably 3 parts by mass to 20 parts by mass with respect to 100 parts by mass of the pigment. The pigment derivative may be used singly or in combination of two or more kinds thereof.

<Polymerizable Compound>

The photosensitive coloring composition according to the embodiment of the present disclosure preferably contains a polymerizable compound. As the polymerizable compound, a known compound which is cross-linkable by a radical, an acid, or heat can be used. In the present disclosure, the polymerizable compound is preferably, for example, a compound having an ethylenically unsaturated group. Examples of the ethylenically unsaturated group include a vinyl group, a (meth)allyl group, and a (meth)acryloyl group. The polymerizable compound used in the present disclosure is preferably a radically polymerizable compound.

Any chemical forms of a monomer, a prepolymer, an oligomer, or the like may be used as the polymerizable compound, but a monomer is preferable. A molecular weight of the polymerizable compound is preferably 100 to 3,000. The upper limit is more preferably 2,000 or less and still more preferably 1,500 or less. The lower limit is more preferably 150 or more and still more preferably 250 or more.

The polymerizable compound is preferably a compound including 3 or more ethylenically unsaturated groups, more preferably a compound including 3 to 15 ethylenically unsaturated groups, and still more preferably a compound having 3 to 6 ethylenically unsaturated groups. In addition, the polymerizable compound is preferably a trifunctional to pentadecafunctional (meth)acrylate compound and more preferably a trifunctional to hexafunctional (meth)acrylate compound. Specific examples of the polymerizable compound include the compounds described in paragraphs 0095 to 0108 of JP2009-288705A, paragraph 0227 of JP2013-029760A, paragraphs 0254 to 0257 of JP2008-292970A, paragraphs 0034 to 0038 of JP2013-253224A, paragraph 0477 of JP2012-208494A, JP2017-048367A, JP6057891B, and JP6031807B, the contents of which are incorporated herein by reference.

As the polymerizable compound, dipentaerythritol triacrylate (as a commercially available product, KAYARAD D-330 manufactured by Nippon Kayaku Co., Ltd.), dipentaerythritol tetraacrylate (as a commercially available product, KAYARAD D-320 manufactured by Nippon Kayaku Co., Ltd.), dipentaerythritol penta(meth)acrylate (as a commercially available product, KAYARAD D-310 manufactured by Nippon Kayaku Co., Ltd.), dipentaerythritol hexa(meth)acrylate (as a commercially available product, KAYARAD DPHA manufactured by Nippon Kayaku Co., Ltd., NK ESTER A-DPH-12E manufactured by Shin-Nakamura Chemical Co., Ltd.), or a compound having a structure in which these (meth)acryloyl groups are bonded through an ethylene glycol and/or a propylene glycol residue (for example, SR454 and SR499 which are commercially available products from Sartomer Company Inc.) is preferable. In addition, as the polymerizable compound, diglycerin ethylene oxide (EO)-modified (meth)acrylate (as a commercially available product, M-460 manufactured by TOAGOSEI CO., LTD.), pentaerythritol tetraacrylate (NK ESTER A-TMMT manufactured by Shin-Nakamura Chemical Co., Ltd.), 1,6-hexanediol diacrylate (KAYARAD HDDA manufactured by Nippon Kayaku Co., Ltd.), RP-1040 (manufactured by Nippon Kayaku Co., Ltd.), ARONIX TO-2349 (manufactured by TOAGOSEI CO., LTD.), NK OLIGO UA-7200 (manufactured by Shin-Nakamura Chemical Co., Ltd.), 8UH-1006 and 8UH-1012 (manufactured by Taisei Fine Chemical Co., Ltd.), Light Acrylate POB-A0 (manufactured by KYOEISHA CHEMICAL Co., Ltd.), and the like can also be used.

As the polymerizable compound, it is also preferable to use a trifunctional (meth)acrylate compound such as trimethylolpropane tri(meth)acrylate, trimethylolpropane propyleneoxide-modified tri(meth)acrylate, trimethylolpropane ethyleneoxide-modified tri(meth)acrylate, isocyanuric acid ethyleneoxide-modified tri(meth)acrylate, and pentaerythritol tri(meth)acrylate. Examples of a commercially available product of the trifunctional (meth)acrylate compound include ARONIX M-309, M-310, M-321, M-350, M-360, M-313, M-315, M-306, M-305, M-303, M-452, and M-450 (manufactured by TOAGOSEI CO., LTD.), NK ESTER A9300, A-GLY-9E, A-GLY-20E, A-TMM-3, A-TMM-3L, A-TMM-3LM-N, A-TMPT, and TMPT (manufactured by Shin-Nakamura Chemical Co., Ltd.), and KAYARAD GPO-303, TMPTA, THE-330, TPA-330, and PET-30 (manufactured by Nippon Kayaku Co., Ltd.).

As the polymerizable compound, a polymerizable compound having an acidic functional group can also be used. By using a polymerizable compound having an acidic functional group, a photosensitive coloring composition in a non-exposed portion is easily removed during development and the generation of the development residue can be suppressed. Examples of the acidic functional group include a carboxy group, a sulfo group, and a phosphoric acidic functional group, and a carboxy group is preferable. Examples of a commercially available product of the polymerizable compound having an acidic functional group include ARONIX M-510, M-520, and ARONIX TO-2349 (manufactured by TOAGOSEI CO., LTD). An acid value of the polymerizable compound having an acidic functional group is preferably 0.1 mgKOH/g to 40 mgKOH/g and more preferably 5 mgKOH/g to 30 mgKOH/g. In a case where the acid value of the polymerizable compound is 0.1 mgKOH/g or more, solubility in a developer is good, and in a case where the acid value of the polymerizable compound is 40 mgKOH/g or less, it is advantageous in production and handling.

As the polymerizable compound, a polymerizable compound having a caprolactone structure can also be used. Examples of the polymerizable compound having a caprolactone structure include DPCA-20, DPCA-30, DPCA-60, and DPCA-120, each of which is commercially available as KAYARAD DPCA series from Nippon Kayaku Co., Ltd.

As the polymerizable compound, a polymerizable compound having an alkyleneoxy group can also be used. The polymerizable compound having an alkyleneoxy group is preferably a polymerizable compound having an ethyleneoxy group and/or a propyleneoxy group, more preferably a polymerizable compound having an ethyleneoxy group, and still more preferably a trifunctional to hexafunctional (meth)acrylate compound having 4 to 20 ethyleneoxy groups. Examples of a commercially available product of the polymerizable compound having an alkyleneoxy group include SR-494 manufactured by Sartomer, which is a tetrafunctional (meth)acrylate having 4 ethyleneoxy groups, and KAYARAD TPA-330, which is a trifunctional (meth)acrylate having 3 isobutyleneoxy groups.

As the polymerizable compound, a polymerizable compound having a fluorene skeleton can also be used. Examples of a commercially available product of the polymerizable compound having a fluorene skeleton include OGSOL EA-0200, EA-0300 (manufactured by Osaka Gas Chemicals Co., Ltd., (meth)acrylate monomer having a fluorene skeleton).

As the polymerizable compound, it is also preferable to use a compound which does not substantially include environmentally regulated substances such as toluene. Examples of a commercially available product of such a compound include KAYARAD DPHA LT and KAYARAD DPEA-12 LT (manufactured by Nippon Kayaku Co., Ltd.).

As the polymerizable compound, urethane acrylates described in JP1973-041708B (JP-S48-041708B), JP1976-037193A (JP-S51-037193A), JP1990-032293B (JP-H02-032293B), and JP1990-016765B (JP-H02-016765B); urethane compounds having an ethylene oxide skeleton described in JP1983-049860B (JP-S58-049860B), JP1981-017654B (JP-S56-017654B), JP1987-039417B (JP-S62-039417B), and JP1987-039418B (JP-S62-039418B); or polymerizable compounds having an amino structure or a sulfide structure in the molecule, described in JP1988-277653A (JP-S63-277653A), JP1988-260909A (JP-S63-260909A), and JP1989-105238A (JP-H01-105238A) can also be preferably used. In addition, as the polymerizable compound, commercially available products such as UA-7200 (manufactured by Shin-Nakamura Chemical Co., Ltd.), DPHA-40H (manufactured by Nippon Kayaku Co., Ltd.), and UA-306H, UA-306T, UA-306I, AH-600, T-600, AI-600, and LINC-202UA (manufactured by KYOEISHA CHEMICAL Co., Ltd.) can also be used.

In a case where the photosensitive coloring composition according to the embodiment of the present disclosure contains a polymerizable compound, a content of the photosensitive coloring composition in the total solid content of the photosensitive coloring composition is preferably 0.1% by mass to 50% by mass. The lower limit is more preferably 0.5% by mass or more and still more preferably 1% by mass or more. The upper limit is more preferably 45% by mass or less and still more preferably 40% by mass or less.

In addition, from the viewpoint of curability, developability, and film-forming property, the total content of the polymerizable compound and the resin in the total solid content of the photosensitive coloring composition is preferably 10% by mass to 65% by mass. The lower limit is more preferably 15% by mass or more, still more preferably 20% by mass or more, and particularly preferably 30% by mass or more. The upper limit is more preferably 60% by mass or less, still more preferably 50% by mass or less, and particularly preferably 40% by mass or less. In addition, the photosensitive composition according to the embodiment of the present disclosure preferably contains 30 parts by mass to 300 parts by mass of the resin with respect to 100 parts by mass of the polymerizable compound. The lower limit is more preferably 50 parts by mass or more and particularly preferably 80 parts by mass or more. The upper limit is more preferably 250 parts by mass or less and particularly preferably 200 parts by mass or less.

In the photosensitive coloring composition according to the embodiment of the present disclosure, the polymerizable compound may be used singly or in a combination of two or more kinds thereof. In a case where two or more kinds thereof are used, the total amount thereof is preferably within the above-described range.

<Photopolymerization Initiator>

The photosensitive coloring composition according to the embodiment of the present disclosure preferably includes a photopolymerization initiator. In particular, in a case where the photosensitive coloring composition according to the embodiment of the present disclosure includes a polymerizable compound, it is preferable that the photosensitive coloring composition according to the embodiment of the present disclosure further includes a photopolymerization initiator. The photopolymerization initiator is not particularly limited, and can be appropriately selected from known photopolymerization initiators. For example, a compound having photosensitivity to light in a range from an ultraviolet range to a visible light range is preferable. The photopolymerization initiator is preferably a photoradical polymerization initiator.

Examples of the photopolymerization initiator include a halogenated hydrocarbon derivative (for example, a compound having a triazine skeleton or a compound having an oxadiazole skeleton), an acylphosphine compound, a hexaarylbiimidazole, an oxime compound, an organic peroxide, a thio compound, a ketone compound, an aromatic onium salt, an α-hydroxyketone compound, and an α-aminoketone compound. From the viewpoint of exposure sensitivity, as the photopolymerization initiator, a compound selected from the group consisting of a trihalomethyltriazine compound, a benzyldimethylketal compound, an α-hydroxyketone compound, an α-aminoketone compound, an acylphosphine compound, a phosphine oxide compound, a metallocene compound, an oxime compound, a triarylimidazole dimer, an onium compound, a benzothiazole compound, a benzophenone compound, an acetophenone compound, a cyclopentadiene-benzene-iron complex, a halomethyl oxadiazole compound, and a 3-aryl-substituted coumarin compound is preferable, a compound selected from the group consisting of an oxime compound, an α-hydroxyketone compound, an α-aminoketone compound, and an acylphosphine compound is more preferable, and an oxime compound is still more preferable. In addition, as the photopolymerization initiator, compounds described in paragraphs 0065 to 0111 of JP2014-130173A, compounds described in JP6301489B, peroxide-based photopolymerization initiators described in MATERIAL STAGE, p. 37 to 60, vol. 19, No. 3, 2019, photopolymerization initiators described in WO2018/221177A, photopolymerization initiators described in WO2018/110179A, photopolymerization initiators described in JP2019-043864A, photopolymerization initiators described in JP2019-044030A, and organic peroxides described in JP2019-167313A, the contents of which are incorporated herein by reference.

Examples of a commercially available product of the α-hydroxyketone compound include Omnirad 184, Omnirad 1173, Omnirad 2959, and Omnirad 127 (all of which are manufactured by IGM Resins B.V), Irgacure 184, Irgacure 1173, Irgacure 2959, and Irgacure 127 (all of which are manufactured by BASF SE). Examples of a commercially available product of the α-aminoketone compound include Omnirad 907, Omnirad 369, Omnirad 369E, and Omnirad 379EG (all of which are manufactured by IGM Resins B.V.), Irgacure 907, Irgacure 369, Irgacure 369E, and Irgacure 379EG (all of which are manufactured by BASF SE). Examples of a commercially available product of the acylphosphine compound include Omnirad 819 and Omnirad TPO (both of which are manufactured by IGM Resins B.V.), Irgacure 819 and Irgacure TPO (both of which are manufactured by BASF SE).

Examples of the oxime compound include the compounds described in JP2001-233842A, the compounds described in JP2000-080068A, the compounds described in JP2006-342166A, the compounds described in J. C. S. Perkin 11 (1979, pp. 1653 to 1660), the compounds described in J. C. S. Perkin 11 (1979, pp. 156 to 162), the compounds described in Journal of Photopolymer Science and Technology (1995, pp. 202 to 232), the compounds described in JP2000-066385A, the compounds described in JP2000-080068A, the compounds described in JP2004-534797A, the compounds described in JP2006-342166A, the compounds described in JP2017-019766A, the compounds described in JP6065596B, the compounds described in WO2015/152153A, the compounds described in WO2017/051680A, the compounds described in JP2017-198865A, the compounds described in paragraphs 0025 to 0038 of WO2017/164127A, and compounds described in WO2013/167515A. Specific examples of the oxime compound include 3-benzoyloxyiminobutane-2-one, 3-acetoxyiminobutane-2-one, 3-propionyloxyiminobutane-2-one, 2-acetoxyiminopentane-3-one, 2-acetoxyimino-1-phenylpropane-1-one, 2-benzoyloxyimino-1-phenylpropane-1-one, 3-(4-toluene sulfonyloxy)iminobutane-2-one, and 2-ethoxycarbonyloxyimino-1-phenylpropane-1-one. Examples of a commercially available product thereof include Irgacure OXE01, Irgacure OXE02, Irgacure OXE03, and Irgacure OXE04 (all of which are manufactured by BASF SE), TR-PBG-304 (manufactured by TRONLY), and ADEKA OPTOMER N-1919 (manufactured by ADEKA Corporation; photopolymerization initiator 2 described in JP2012-014052A). In addition, as the oxime compound, it is also preferable to use a compound having no colorability or a compound having high transparency and being resistant to discoloration. Examples of a commercially available product include ADEKA ARKLS NCI-730, NCI-831, and NCI-930 (all of which are manufactured by ADEKA Corporation).

An oxime compound having a fluorene ring can also be used as the photopolymerization initiator. Specific examples of the oxime compound having a fluorene ring include the compounds described in JP2014-137466A.

As the photopolymerization initiator, an oxime compound having a skeleton in which at least one benzene ring of a carbazole ring is a naphthalene ring can also be used. Specific examples of such an oxime compound include the compounds described in WO2013/083505A.

An oxime compound having a fluorine atom can also be used as the photopolymerization initiator. Specific examples of the oxime compound having a fluorine atom include the compounds described in JP2010-262028A, the compounds 24, and 36 to 40 described in JP2014-500852A, and the compound (C-3) described in JP2013-164471A.

An oxime compound having a nitro group can be used as the photopolymerization initiator. The oxime compound having a nitro group is also preferably used in the form of a dimer. Specific examples of the oxime compound having a nitro group include the compounds described in paragraphs 0031 to 0047 of JP2013-114249A and paragraphs 0008 to 0012 and 0070 to 0079 of JP2014-137466A, the compounds described in paragraphs 0007 to 0025 of JP4223071B, and ADEKA ARKLS NCI-831 (manufactured by ADEKA Corporation).

An oxime compound having a benzofuran skeleton can also be used as the photopolymerization initiator. Specific examples thereof include OE-01 to OE-75 described in WO2015/036910A.

In the present invention, as the photopolymerization initiator, an oxime compound in which a substituent having a hydroxy group is bonded to a carbazole skeleton can also be used. Examples of such a photopolymerization initiator include compounds described in WO2019/088055A.

Specific examples of the oxime compound which are preferably used in the present disclosure are shown below, but the present disclosure is not limited thereto.

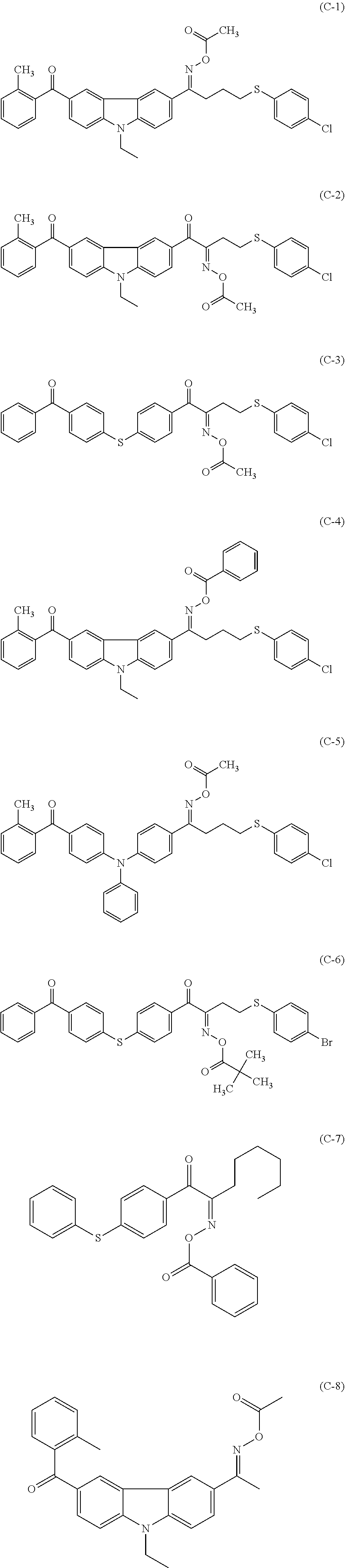

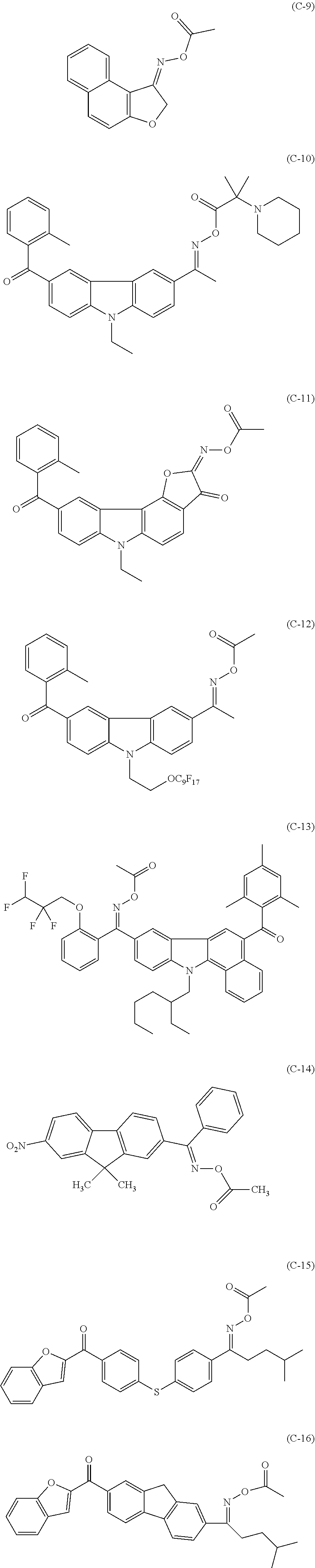

The oxime compound is preferably a compound having a maximal absorption wavelength in a wavelength range of 350 nm to 500 nm and more preferably a compound having a maximal absorption wavelength in a wavelength range of 360 nm to 480 nm. In addition, from the viewpoint of sensitivity, the molar absorption coefficient of the oxime compound at a wavelength of 365 nm or 405 nm is preferably high, more preferably 1,000 to 300,000, still more preferably 2,000 to 300,000, and particularly preferably 5,000 to 200,000. The molar absorption coefficient of a compound can be measured using a known method. For example, the molar absorption coefficient is preferably measured by a spectrophotometer (Cary-5 spectrophotometer, manufactured by Varian Medical Systems, Inc.) using ethyl acetate at a concentration of 0.01 g/L.

As the photopolymerization initiator, a bifunctional or tri- or higher functional photoradical polymerization initiator may be used. By using such a photoradical polymerization initiator, two or more radicals are generated from one molecule of the photoradical polymerization initiator, and as a result, good sensitivity is obtained. In addition, in a case of using a compound having an asymmetric structure, crystallinity is reduced so that solubility in a solvent or the like is improved, precipitation is to be difficult over time, and temporal stability of the photosensitive coloring composition can be improved. Specific examples of the bifunctional or tri- or higher functional photoradical polymerization initiator include dimers of the oxime compounds described in JP2010-527339A, JP2011-524436A, WO2015/004565A, paragraphs 0407 to 0412 of JP2016-532675A, and paragraphs 0039 to 0055 of WO2017/033680A; the compound (E) and compound (G) described in JP2013-522445A; Cmpd 1 to 7 described in WO2016/034963A; the oxime ester photoinitiators described in paragraph 0007 of JP2017-523465A; the photoinitiators described in paragraphs 0020 to 0033 of JP2017-167399A; the photopolymerization initiator (A) described in paragraphs 0017 to 0026 of JP2017-151342A; and the oxime ester photoinitiators described in JP6469669B.

In a case where the photosensitive coloring composition according to the embodiment of the present disclosure contains a photopolymerization initiator, a content of the photopolymerization initiator in the total solid content of the coloring composition is preferably 0.1% by mass to 30% by mass. The lower limit is more preferably 0.5% by mass or more and particularly preferably 1% by mass or more. The upper limit is more preferably 20% by mass or less and particularly preferably 15% by mass or less. In the photosensitive coloring composition according to the embodiment of the present disclosure, the photopolymerization initiator may be used singly or in a combination of two or more kinds thereof. In a case where two or more kinds thereof are used, the total amount thereof is preferably within the above-described range.

<Compound Having Cyclic Ether Group>

The photosensitive coloring composition according to the embodiment of the present disclosure can contain a compound having a cyclic ether group. Examples of the cyclic ether group include an epoxy group and an oxetanyl group. The compound having a cyclic ether group is preferably a compound having an epoxy group. Examples of the compound having an epoxy group include a compound having one or more epoxy groups in one molecule, and a compound two or more epoxy groups in one molecule is preferable. It is preferable to have 1 to 100 epoxy groups in one molecule. The upper limit of the number of epoxy groups may be, for example, 10 or less or 5 or less. The lower limit of the epoxy group is preferably 2 or more. As the compound having an epoxy group, the compounds described in paragraphs 0034 to 0036 of JP2013-011869A, paragraphs 0147 to 0156 of JP2014-043556A, and paragraphs 0085 to 0092 of JP2014-089408A, and the compounds described in JP2017-179172A can also be used. The contents of the publications are incorporated herein by reference.

The compound having an epoxy group may be a low-molecular-weight compound (for example, having a molecular weight of less than 2,000, and further, a molecular weight of less than 1,000) or a high-molecular-weight compound (macromolecule) (for example, having a molecular weight of 1,000 or more, and in a case of a polymer, having a weight-average molecular weight of 1,000 or more). The weight-average molecular weight of the compound having an epoxy group is preferably 200 to 100,000 and more preferably 500 to 50,000. The upper limit of the weight-average molecular weight is still more preferably 10,000 or less, particularly preferably 5,000 or less, and most preferably 3,000 or less.

As the compound having an epoxy group, an epoxy resin can be preferably used. Examples of the epoxy resin include an epoxy resin which is a glycidyl etherified product of a phenol compound, an epoxy resin which is a glycidyl etherified product of various novolac resins, an alicyclic epoxy resin, an aliphatic epoxy resin, a heterocyclic epoxy resin, a glycidyl ester-based epoxy resin, a glycidyl amine-based epoxy resin, an epoxy resin obtained by glycidylating halogenated phenols, a condensate of a silicon compound having an epoxy group and another silicon compound, and a copolymer of a polymerizable unsaturated compound having an epoxy group and another polymerizable unsaturated compound. An epoxy equivalent of the epoxy resin is preferably 310 g/eq to 3,300 g/eq, more preferably 310 g/eq to 1,700 g/eq, and still more preferably 310 g/eq to 1,000 g/eq.

Examples of a commercially available product of the compound having a cyclic ether group include EHPE 3150 (manufactured by Daicel Corporation), EPICLON N-695 (manufactured by DIC Corporation), and MARPROOF G-0150M, G-0105SA, G-0130SP, G-0250SP, G-1005S, G-1005SA, G-110S, G-2050M, G-01100, and G-01758 (all of which are manufactured by NOF Corporation, an epoxy group-containing polymer).

In a case where the photosensitive coloring composition according to the embodiment of the present disclosure contains a compound having a cyclic ether group, a content of the compound having a cyclic ether group in the total solid content of the photosensitive coloring composition is preferably 0.1% by mass to 20% by mass. The lower limit is more preferably 0.5% by mass or more and particularly preferably 1% by mass or more. The upper limit is more preferably 15% by mass or less and particularly preferably 10% by mass or less. In the coloring composition according to the embodiment of the present disclosure, the compound having a cyclic ether group may be used singly or in combination of two or more kinds thereof. In a case where two or more kinds thereof are used, the total amount thereof is preferably within the above-described range.

<Silane Coupling Agent>

The photosensitive coloring composition according to the embodiment of the present disclosure can contain a silane coupling agent. According to this aspect, adhesiveness of a film to be obtained with a support can be further improved. In the present disclosure, the silane coupling agent means a silane compound having a hydrolyzable group and other functional groups. In addition, the hydrolyzable group refers to a substituent directly linked to a silicon atom and capable of forming a siloxane bond due to at least one of a hydrolysis reaction or a condensation reaction. Examples of the hydrolyzable group include a halogen atom, an alkoxy group, and an acyloxy group, and an alkoxy group is preferable. That is, it is preferable that the silane coupling agent is a compound having an alkoxysilyl group. Examples of the functional group other than the hydrolyzable group include a vinyl group, a (meth)allyl group, a (meth)acryloyl group, a mercapto group, an epoxy group, an oxetanyl group, an amino group, a ureido group, a sulfide group, an isocyanate group, and a phenyl group, and an amino group, a (meth)acryloyl group, or an epoxy group is preferable. Specific examples of the silane coupling agent include N-β-aminoethyl-γ-aminopropyl methyldimethoxysilane (trade name: KBM-602, manufactured by Shin-Etsu Chemical Co., Ltd.), N-β-aminoethyl-γ-aminopropyl trimethoxysilane (trade name: KBM-603, manufactured by Shin-Etsu Chemical Co., Ltd.), N-β-aminoethyl-γ-aminopropyl triethoxysilane (trade name: KBE-602, manufactured by Shin-Etsu Chemical Co., Ltd.), γ-aminopropyl trimethoxysilane (trade name: KBM-903, manufactured by Shin-Etsu Chemical Co., Ltd.), γ-aminopropyl triethoxysilane (trade name: KBE-903, manufactured by Shin-Etsu Chemical Co., Ltd.), 3-methacryloxypropylmethyl dimethoxysilane (trade name: KBM-502, manufactured by Shin-Etsu Chemical Co., Ltd.), and 3-methacryloxypropyl trimethoxysilane (trade name: KBM-503, manufactured by Shin-Etsu Chemical Co., Ltd.). In addition, specific examples of the silane coupling agent include the compounds described in paragraphs 0018 to 0036 of JP2009-288703A and the compounds described in paragraphs 0056 to 0066 of JP2009-242604A, the contents of which are incorporated herein by reference.

In a case where the photosensitive coloring composition according to the embodiment of the present disclosure contains a silane coupling agent, a content of the silane coupling agent in the total solid content of the photosensitive composition is preferably 0.1% by mass to 5% by mass. The upper limit is more preferably 3% by mass or less and particularly preferably 2% by mass or less. The lower limit is more preferably 0.5% by mass or more and particularly preferably 1% by mass or more. In the photosensitive coloring composition according to the embodiment of the present disclosure, the silane coupling agent may be used singly or in combination of two or more kinds thereof. In a case where two or more kinds thereof are used, the total amount thereof is preferably within the above-described range.

<Organic Solvent>

The photosensitive coloring composition according to the embodiment of the present disclosure preferably contains an organic solvent. Examples of the organic solvent include an ester-based solvent, a ketone-based solvent, an alcohol-based solvent, an amide-based solvent, an ether-based solvent, and a hydrocarbon-based solvent. The details of the organic solvent can be found in paragraph 0223 of WO2015/166779A, the content of which is incorporated herein by reference. In addition, an ester-based solvent in which a cyclic alkyl group is substituted or a ketone-based solvent in which a cyclic alkyl group is substituted can also be preferably used. Specific examples of the organic solvent include polyethylene glycol monomethyl ether, dichloromethane, methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, cyclohexyl acetate, cyclopentanone, ethyl carbitol acetate, butyl carbitol acetate, propylene glycol monomethyl ether, propylene glycol monomethyl ether acetate, 3-methoxy-N,N-dimethylpropanamide, and 3-butoxy-N,N-dimethylpropanamide. In this case, it may be preferable that the content of aromatic hydrocarbons (such as benzene, toluene, xylene, and ethylbenzene) as the organic solvent is low (for example, 50 parts per million (ppm) by mass or less, 10 ppm by mass or less, or 1 ppm by mass or less with respect to the total amount of the organic solvent) in consideration of environmental aspects and the like.

In the present disclosure, an organic solvent having a low metal content is preferably used. For example, the metal content in the organic solvent is preferably 10 mass parts per billion (ppb) or less. Optionally, an organic solvent having a metal content at a mass parts per trillion (ppt) level may be used. For example, such an organic solvent is available from Toyo Gosei Co., Ltd. (The Chemical Daily, Nov. 13, 2015).

Examples of a method for removing impurities such as a metal from the organic solvent include distillation (such as molecular distillation and thin-film distillation) and filtration using a filter. The filter pore size of the filter used for the filtration is preferably 10 μm or less, more preferably 5 μm or less, and still more preferably 3 μm or less. As a material of the filter, polytetrafluoroethylene, polyethylene, or nylon is preferable.

The organic solvent may include an isomer (a compound having the same number of atoms and a different structure). In addition, only one kind of isomers may be included, or a plurality of isomers may be included.

In the present disclosure, the organic solvent preferably has the content of peroxides of 0.8 mmol/L or less, and more preferably, the organic solvent does not substantially include peroxides.

A content of the organic solvent in the photosensitive coloring composition is preferably 20% by mass to 95% by mass, more preferably 30% by mass to 90% by mass, and still more preferably 40% by mass to 90% by mass.

In addition, from the viewpoint of environmental regulation, it is preferable that the photosensitive coloring composition according to the embodiment of the present disclosure does not substantially contain environmentally regulated substances. In the present disclosure, the description “does not substantially contain environmentally regulated substances” means that the content of the environmentally regulated substances in the photosensitive coloring composition is 50 ppm by mass or less, preferably 30 ppm by mass or less, more preferably 10 ppm by mass or less, and particularly preferably 1 ppm by mass or less. Examples of the environmentally regulated substances include benzenes; alkylbenzenes such as toluene and xylene; and halogenated benzenes such as chlorobenzene. These compounds are registered as environmentally regulated substances in accordance with Registration Evaluation Authorization and Restriction of CHemicals (REACH) rules, Pollutant Release and Transfer Register (PRTR) law, Volatile Organic Compounds (VOC) regulation, and the like, and strictly regulated in their usage and handling method. These compounds can be used as a solvent in a case of producing respective components used in the photosensitive coloring composition according to the embodiment of the present disclosure, and may be incorporated into the photosensitive coloring composition as a residual solvent. From the viewpoint of human safety and environmental considerations, it is preferable to reduce these substances as much as possible. Examples of a method for reducing the environmentally regulated substances include a method for reducing the environmentally regulated substances by distilling the environmentally regulated substances from a system by heating or depressurizing the system such that the temperature of the system is higher than a boiling point of the environmentally regulated substances. In addition, in a case of distilling a small amount of the environmentally regulated substances, it is also useful to azeotrope with a solvent having the boiling point equivalent to that of the above-described solvent in order to increase efficiency. In addition, in a case of containing a compound having radical polymerizability, in order to suppress the radical polymerization reaction proceeding during the distillation under reduced pressure to cause crosslinking between the molecules, a polymerization inhibitor or the like may be added and the distillation under reduced pressure is performed. These distillation methods can be performed at any stage of raw material, product (for example, resin solution after polymerization or polyfunctional monomer solution) obtained by reacting the raw material, photosensitive coloring composition produced by mixing these compounds, or the like.

<Polymerization Inhibitor>

The photosensitive coloring composition according to the embodiment of the present disclosure can contain a polymerization inhibitor. Examples of the polymerization inhibitor include hydroquinone, p-methoxyphenol, di-tert-butyl-p-cresol, pyrogallol, tert-butyl catechol, benzoquinone, 4,4′-thiobis(3-methyl-6-tert-butylphenol), 2,2′-methylenebis(4-methyl-6-t-butylphenol), and an N-nitrosophenylhydroxylamine salt (an ammonium salt, a cerous salt, or the like). Among these, p-methoxyphenol is preferable. A content of the polymerization inhibitor in the total solid content of the photosensitive coloring composition is preferably 0.0001% by mass to 5% by mass.

<Surfactant>

The photosensitive coloring composition according to the embodiment of the present disclosure can contain a surfactant. As the surfactant, various surfactants such as a fluorine-based surfactant, a nonionic surfactant, a cationic surfactant, an anionic surfactant, and a silicone-based surfactant can be used. Examples of the surfactant include surfactants described in paragraphs 0238 to 0245 of WO2015/166779A, the contents of which are incorporated herein by reference.

In the present disclosure, it is preferable that the surfactant is a fluorine-based surfactant. By containing a fluorine-based surfactant in the photosensitive coloring composition, liquid characteristics (particularly, fluidity) are further improved, and liquid saving properties can be further improved. In addition, it is possible to form a film with a small thickness unevenness.

A fluorine content in the fluorine-based surfactant is preferably 3% by mass to 40% by mass, more preferably 5% by mass to 30% by mass, and particularly preferably 7% by mass to 25% by mass. The fluorine-based surfactant in which the fluorine content is within the above-described range is effective in terms of the evenness of the thickness of the coating film or liquid saving properties and the solubility of the surfactant in the photosensitive coloring composition is also good.

Examples of the fluorine-based surfactant include surfactants described in paragraphs 0060 to 0064 of JP2014-041318A (paragraphs 0060 to 0064 of the corresponding WO2014/017669A) and the like, and surfactants described in paragraphs 0117 to 0132 of JP2011-132503A, the contents of which are incorporated herein by reference. Examples of a commercially available product of the fluorine-based surfactant include: MEGAFACE F171, F172, F173, F176, F177, F141, F142, F143, F144, R30, F437, F475, F479, F482, F554, F780, EXP, and MFS-330 (all of which are manufactured by DIC Corporation); FLUORAD FC430, FC431, and FC171 (all of which are manufactured by Sumitomo 3M Ltd.); SURFLON S-382, SC-101, SC-103, SC-104, SC-105, SC-1068, SC-381, SC-383, S-393, and KH-40 (all of which are manufactured by Asahi Glass Co., Ltd.); and POLYFOX PF636, PF656, PF6320, PF6520, and PF7002 (all of which are manufactured by OMNOVA Solutions Inc.).

In addition, as the fluorine-based surfactant, an acrylic compound, which has a molecular structure having a functional group containing a fluorine atom and in which, by applying heat to the molecular structure, the functional group containing a fluorine atom is broken to volatilize a fluorine atom, can also be suitably used. Examples of such a fluorine-based surfactant include MEGAFACE DS series manufactured by DIC Corporation (The Chemical Daily, Feb. 22, 2016; Nikkei Business Daily, Feb. 23, 2016) such as MEGAFACE DS-21.

In addition, it is also preferable that a polymer of a fluorine atom-containing vinyl ether compound having a fluorinated alkyl group or a fluorinated alkylene ether group, and a hydrophilic vinyl ether compound is used as the fluorine-based surfactant. With regard to such a fluorine-based surfactant, reference can be made to the description in JP2016-216602A, the contents of which are incorporated herein by reference.

A block polymer can also be used as the fluorine-based surfactant. Examples thereof include the compounds described in JP2011-089090A. As the fluorine-based surfactant, a fluorine-containing polymer compound including a repeating unit derived from a (meth)acrylate compound having a fluorine atom and a repeating unit derived from a (meth)acrylate compound having 2 or more (preferably 5 or more) alkyleneoxy groups (preferably ethyleneoxy groups or propyleneoxy groups) can also be preferably used. In addition, fluorine-containing surfactants described in paragraphs 0016 to 0037 of JP2010-032698A, or the following compounds are also exemplified as the fluorine-based surfactant used in the present disclosure.

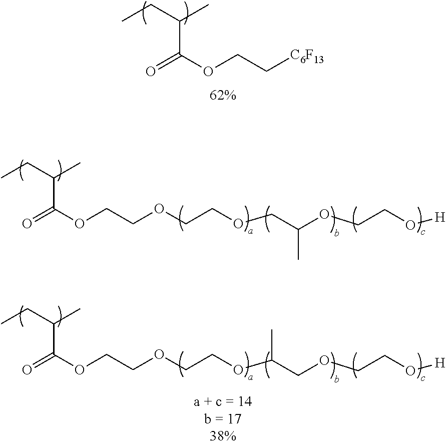

A weight-average molecular weight of the compound is preferably 3,000 to 50,000 and, for example, 14,000. In the compound, “%” representing the proportion of a repeating unit is mol %.

In addition, as the fluorine-based surfactant, a fluorine-containing polymer having an ethylenically unsaturated group in the side chain can be used. Specific examples thereof include compounds described in paragraphs 0050 to 0090 and paragraphs 0289 to 0295 of JP2010-164965A, for example, MEGAFACE RS-101, RS-102, RS-718K, and RS-72-K manufactured by DIC Corporation. In addition, as the fluorine-based surfactant, compounds described in paragraphs 0015 to 0158 of JP2015-117327A can also be used.

Examples of the nonionic surfactant include glycerol, trimethylolpropane, trimethylolethane, an ethoxylate and propoxylate thereof (for example, glycerol propoxylate or glycerol ethoxylate), polyoxyethylene lauryl ether, polyoxyethylene stearyl ether, polyoxyethylene oleyl ether, polyoxyethylene octylphenyl ether, polyoxyethylene nonylphenyl ether, polyethylene glycol dilaurate, polyethylene glycol distearate, sorbitan fatty acid esters, PLURONIC L10, L31, L61, L62, 10R5, 17R2, and 25R2 (manufactured by BASF SE), TETRONIC 304, 701, 704, 901, 904, and 150R1 (manufactured by BASF SE), SOLSPERSE 20000 (manufactured by Lubrizol Japan Ltd.), NCW-101, NCW-1001, and NCW-1002 (all of which are manufactured by FUJIFILM Wako Pure Chemical Corporation), PIONIN D-6112, D-6112-W, and D-6315 (all of which are manufactured by Takemoto Oil & Fat Co., Ltd.), and OLFINE E1010 and SURFYNOL 104, 400, and 440 (all of which are manufactured by Nissin Chemical Co., Ltd.).

Examples of the silicone-based surfactant include TORAY SILICONE DC3PA, TORAY SILICONE SH7PA, TORAY SILICONE DC11PA, TORAY SILICONE SH21PA, TORAY SILICONE SH28PA, TORAY SILICONE SH29PA, TORAY SILICONE SH30PA, and TORAY SILICONE SH8400 (all of which are manufactured by Dow Corning Toray Co., Ltd.), TSF-4440, TSF-4300, TSF-4445, TSF-4460, and TSF-4452 (all of which are manufactured by Momentive Performance Materials Co., Ltd.), KP-341, KF-6001, and KF-6002 (all of which are manufactured by Shin-Etsu Silicone Co., Ltd.), and BYK307, BYK323, and BYK330 (all of which are manufactured by BYK Chemie).

A content of the surfactant in the total solid content of the photosensitive coloring composition is preferably 0.001% by mass to 5.0% by mass and more preferably 0.005% by mass to 3.0% by mass. In the photosensitive coloring composition according to the embodiment of the present disclosure, the surfactant may be used singly or in a combination of two or more kinds thereof. In a case where two or more kinds thereof are used, the total amount thereof is preferably within the above-described range.

<Ultraviolet Absorber>

The photosensitive coloring composition according to the embodiment of the present disclosure can contain an ultraviolet absorber. As the ultraviolet absorber, a conjugated diene compound, an aminodiene compound, a salicylate compound, a benzophenone compound, a benzotriazole compound, an acrylonitrile compound, a hydroxyphenyltriazine compound, an indole compound, a triazine compound, or the like can be used. Examples of details thereof include compounds described in paragraphs 0052 to 0072 of JP2012-208374A, paragraphs 0317 to 0334 of JP2013-068814A, and paragraphs 0061 to 0080 of JP2016-162946A, the contents of which are incorporated herein by reference. Examples of a commercially available product of the ultraviolet absorber include UV-503 (manufactured by Daito Chemical Co., Ltd). In addition, examples of the benzotriazole compound include MYUA series manufactured by Miyoshi Oil & Fat Co., Ltd. (The Chemical Daily, Feb. 1, 2016). In addition, as the ultraviolet absorber, compounds described in paragraphs 0049 to 0059 of JP6268967B can also be used.

A content of the ultraviolet absorber in the total solid content of the photosensitive coloring composition is preferably 0.010% by mass to 10% by mass and more preferably 0.010% by mass to 5% by mass. In the photosensitive coloring composition according to the embodiment of the present disclosure, the ultraviolet absorber may be used singly or in a combination of two or more kinds thereof. In a case where two or more kinds thereof are used, the total amount thereof is preferably within the above-described range.

<Antioxidant>

The photosensitive coloring composition according to the embodiment of the present disclosure can contain an antioxidant. Examples of the antioxidant include a phenol compound, a phosphite ester compound, and a thioether compound. As the phenol compound, any phenol compound which is known as a phenol-based antioxidant can be used. Preferred examples of the phenol compound include a hindered phenol compound. A compound having a substituent at a site (ortho position) adjacent to a phenolic hydroxy group is preferable. As the substituent, a substituted or unsubstituted alkyl group having 1 to 22 carbon atoms is preferable. In addition, as the antioxidant, a compound having a phenol group and a phosphite ester group in the same molecule is also preferable. In addition, as the antioxidant, a phosphorus antioxidant can also be suitably used. Examples of the phosphorus antioxidant include tris[2-[[2,4,8,10-tetrakis(1,1-dimethylethyl)dibenzo[d,f][1,3,2]dioxaphosphepin-6-yl]oxy]ethyl]amine, tris[2-[(4,6,9,11-tetra-tert-butyldibenzo[d,f][1,3,2]dioxaphosphepin-2-yl)oxy]ethyl]amine, and ethyl bis(2,4-di-tert-butyl-6-methylphenyl)phosphite. Examples of a commercially available product of the antioxidant include ADK STAB AO-20, ADK STAB AO-30, ADK STAB AO-40, ADK STAB AO-50, ADK STAB AO-50F, ADK STAB AO-60, ADK STAB AO-60G, ADK STAB AO-80, and ADK STAB AO-330 (all of which are manufactured by ADEKA Corporation). In addition, as the antioxidant, compounds described in paragraphs 0023 to 0048 of JP6268967B, compounds described in KR10-2019-0059371A, and the like can also be used.

A content of the antioxidant in the total solid content of the photosensitive coloring composition is preferably 0.01% by mass to 20% by mass and more preferably 0.3% by mass to 15% by mass. In the photosensitive coloring composition according to the embodiment of the present disclosure, the antioxidant may be used singly or in a combination of two or more kinds thereof. In a case where two or more kinds thereof are used, the total amount thereof is preferably within the above-described range.

<Other Components>

Optionally, the photosensitive coloring composition according to the embodiment of the present disclosure may further contain a sensitizer, a curing accelerator, a filler, a thermal curing accelerator, a plasticizer, and other auxiliary agents (for example, conductive particles, an antifoaming agent, a flame retardant, a leveling agent, a peeling accelerator, an aromatic chemical, a surface tension adjuster, or a chain transfer agent). By appropriately containing these components, properties such as film properties can be adjusted. The details of the components can be found in, for example, paragraph 0183 of JP2012-003225A (corresponding to paragraph 0237 of US2013/0034812A) and paragraphs 0101 to 0104 and 0107 to 0109 of JP2008-250074A, the contents of which are incorporated herein by reference. In addition, optionally, the coloring composition according to the embodiment of the present disclosure may contain a potential antioxidant. Examples of the potential antioxidant include a compound in which a site functioning as an antioxidant is protected by a protective group, and the protective group is eliminated by heating the compound at 100° C. to 250° C. or heating the compound at 80° C. to 200° C. in the presence of an acid or base catalyst so that the compound functions as an antioxidant. Examples of the potential antioxidant include compounds described in WO2014/021023A, WO2017/030005A, and JP2017-008219A. Examples of a commercially available product of the potential antioxidant include ADEKA ARKLS GPA-5001 (manufactured by ADEKA Corporation). In addition, as described in JP2018-155881A, C. I. Pigment Yellow 129 may be added for the purpose of improving weather fastness.

In order to adjust the refractive index of a film to be obtained, the photosensitive coloring composition according to the embodiment of the present disclosure may contain a metal oxide. Examples of the metal oxide include TiO₂, ZrO₂, Al₂O₃, and SiO₂. A primary particle diameter of the metal oxide is preferably 1 nm to 100 nm, more preferably 3 nm to 70 nm, and particularly preferably 5 nm to 50 nm. The metal oxide may have a core-shell structure. In addition, in this case, the core portion may be hollow.

In addition, the photosensitive coloring composition according to the embodiment of the present disclosure may include a light-resistance improver. Examples of the light-resistance improver include the compounds described in paragraphs 0036 and 0037 of JP2017-198787A, the compounds described in paragraphs 0029 to 0034 of JP2017-146350A, the compounds described in paragraphs 0036 and 0037, and 0049 to 0052 of JP2017-129774A, the compounds described in paragraphs 0031 to 0034, 0058, and 0059 of JP2017-129674A, the compounds described in paragraphs 0036 and 0037, and 0051 to 0054 of JP2017-122803A, the compounds described in paragraphs 0025 to 0039 of WO2017/164127A, the compounds described in paragraphs 0034 to 0047 of JP2017-186546A, the compounds described in paragraphs 0019 to 0041 of JP2015-025116A, the compounds described in paragraphs 0101 to 0125 of JP2012-145604A, the compounds described in paragraphs 0018 to 0021 of JP2012-103475A, the compounds described in paragraphs 0015 to 0018 of JP2011-257591A, the compounds described in paragraphs 0017 to 0021 of JP2011-191483A, the compounds described in paragraphs 0108 to 0116 of JP2011-145668A, and the compounds described in paragraphs 0103 to 0153 of JP2011-253174A.

In the photosensitive coloring composition according to the embodiment of the present disclosure, the content of liberated metals which are not bonded to or coordinated with a pigment or the like is preferably 100 ppm or less, more preferably 50 ppm or less, and still more preferably 10 ppm or less, it is particularly preferable to not contain the liberated metals substantially. According to this aspect, effects such as stabilization of pigment dispersibility (restraint of aggregation), improvement of spectral characteristics due to improved dispersibility, restraint of conductivity fluctuation due to stabilization of curable components or elution of metal atoms and metal ions, and improvement of display characteristics can be expected. In addition, the effects described in JP2012-153796A, JP2000-345085A, JP2005-200560A, JP1996-043620A (JP-H08-043620A), JP2004-145078A, JP2014-119487A, JP2010-083997A, JP2017-090930A, JP2018-025612A, JP2018-025797A, JP2017-155228A, JP2018-036521A, and the like can be obtained. Examples of the types of the above-described liberated metals include Na, K, Ca, Sc, Ti, Mn, Cu, Zn, Fe, Cr, Co, Mg, Al, Sn, Zr, Ga, Ge, Ag, Au, Pt, Cs, Ni, Cd, Pb, and Bi. In addition, in the photosensitive coloring composition according to the embodiment of the present disclosure, the content of liberated halogen which is not bonded to or coordinated with a pigment or the like is preferably 100 ppm or less, more preferably 50 ppm or less, and still more preferably 10 ppm or less, it is particularly preferable to not contain the liberated halogen substantially. Examples of halogen include F, Cl, Br, I, and anions thereof. Examples of a method for reducing liberated metals and halogens in the photosensitive coloring composition include washing with ion exchange water, filtration, ultrafiltration, and purification with an ion exchange resin.

In addition, the photosensitive coloring composition according to the embodiment of the present disclosure may include a dye. As the dye, a known dye can be used.

The dye is not particularly limited, and examples thereof include a pyrazoleazo compound, an anilinoazo compound, a triarylmethane compound, an anthraquinone compound, an anthrapyridone compound, a benzylidene compound, an oxonol compound, a pyrazolotriazoleazo compound, a pyridoneazo compound, a cyanine compound, a phenothiazine compound, a pyrrolopyrazoleazomethine compound, a xanthene compound, a phthalocyanine compound, a benzopyran compound, an indigo compound, and a pyrromethene compound.

In addition, as the dye, methine dyes described in JP2019-073695A, methine dyes described in JP2019-073696A, methine dyes described in JP2019-073697A, and methine dyes described in JP2019-073698A can also be used.

In the photosensitive coloring composition according to the embodiment of the present disclosure, a coloring agent multimer can also be used. The coloring agent multimer is preferably a dye which is used after being dissolved in a solvent. In addition, the coloring agent multimer may form a particle. In a case where the coloring agent multimer is a particle, the coloring agent multimer is usually used in a state of being dispersed in a solvent. The coloring agent multimer in the particle state can be obtained by, for example, emulsion polymerization, and specific examples thereof include the compounds and production methods described in JP2015-214682A. The coloring agent multimer has two or more coloring agent structures in one molecule, and preferably has three or more coloring agent structures in one molecule. The upper limit is particularly not limited, but may be 100 or less. A plurality of coloring agent structures included in one molecule may be the same coloring agent structure or different coloring agent structures. A weight-average molecular weight (Mw) of the coloring agent multimer is preferably 2,000 to 50,000. The lower limit is more preferably 3,000 or more and still more preferably 6,000 or more. The upper limit is more preferably 30,000 or less and still more preferably 20,000 or less. As the coloring agent multimer, the compounds described in JP2011-213925A, JP2013-041097A, JP2015-028144A, JP2015-030742A, WO2016/031442A, or the like can also be used.

It is preferable that the content of the dye is smaller than the content of the pigment.

It is also preferable that the photosensitive coloring composition according to the embodiment of the present disclosure does not substantially include terephthalic acid ester.

The moisture content of the photosensitive coloring composition according to the embodiment of the present disclosure is preferably 3% by mass or less, more preferably 0.01% by mass to 1.5% by mass, and particularly preferably 0.1% by mass to 1.0% by mass. The moisture content can be measured by a Karl Fischer method.

The photosensitive coloring composition according to the embodiment of the present disclosure can be used after viscosity is adjusted for the purposes of adjusting the state of a film surface (flatness or the like), adjusting a film thickness, or the like. The value of the viscosity can be appropriately selected as desired, and is, for example, preferably 0.3 mPa·s to 50 mPa·s, and more preferably 0.5 mPa·s to 20 mPa·s at 23° C. As for a method for measuring the viscosity, the viscosity can be measured, for example, with a temperature being adjusted to 23° C., using a viscometer RE85L (rotor: 1°34′×R24, measurement range of 0.6 to 1,200 mPa·s) manufactured by Toki Sangyo Co., Ltd.

In a case where the photosensitive coloring composition according to the embodiment of the present disclosure is used as a color filter in applications for a liquid crystal display device, a voltage holding ratio of a liquid crystal display element including the color filter is preferably 70% or more and more preferably 90% or more. A known method for obtaining a high voltage holding ratio can be appropriately incorporated, and examples of typical methods include use of high-purity materials (for example, reduction in ionic impurities) and control of the amount of acidic functional groups in a composition. The voltage holding ratio can be measured by, for example, the methods described in paragraph 0243 of JP2011-008004A and paragraphs 0123 to 0129 of JP2012-224847A.

<Storage Container>

A storage container for the photosensitive coloring composition according to the embodiment of the present disclosure is not particularly limited, and a known storage container can be used. In addition, as the storage container, it is also preferable to use a multilayer bottle having an interior wall constituted with six layers from six kinds of resins or a bottle having a 7-layer structure from 6 kinds of resins for the purpose of suppressing infiltration of impurities into raw materials or photosensitive coloring compositions. Examples of such a container include the containers described in JP2015-123351A. In addition, for the purpose of preventing metal elution from the container interior wall, improving storage stability of the composition, and suppressing the alteration of components, it is also preferable that an interior wall of the photosensitive coloring composition is formed of glass, stainless steel, or the like. Storage conditions of the photosensitive coloring composition according to the embodiment of the present disclosure is not particularly limited, and a known method in the related art can be used. In addition, a method described in JP2016-180058A can be used.

<Method for Preparing Photosensitive Coloring Composition>

The photosensitive coloring composition according to the embodiment of the present disclosure can be prepared by mixing the above-described components with each other. In the preparation of the photosensitive coloring composition, all the components may be dissolved and/or dispersed at the same time in a solvent to prepare the photosensitive coloring composition, or the respective components may be appropriately left in two or more solutions or dispersion liquids and mixed to prepare the photosensitive coloring composition upon use (during coating), as desired.

In addition, in the preparation of the photosensitive coloring composition, a process for dispersing the pigment is also preferably included. In the process for dispersing the pigment, examples of a mechanical force which is used for dispersing the pigment include compression, pressing, impact, shear, and cavitation. Specific examples of these processes include a beads mill, a sand mill, a roll mill, a ball mill, a paint shaker, a microfluidizer, a high-speed impeller, a sand grinder, a flow jet mixer, high-pressure wet atomization, and ultrasonic dispersion. In addition, in the pulverization of the pigment in a sand mill (beads mill), it is preferable to perform a treatment under the condition for increasing a pulverization efficiency by using beads having small diameters; increasing the filling rate of the beads; or the like. Incidentally, it is preferable to remove coarse particles by filtration, centrifugation, or the like after the pulverization treatment. In addition, as the process and the dispersing machine for dispersing the pigment, the process and the dispersing machine described in “Dispersion Technology Comprehension, published by Johokiko Co., Ltd., Jul. 15, 2005”, “Actual comprehensive data collection on dispersion technology and industrial application centered on suspension (solid/liquid dispersion system), published by Publication Department, Management Development Center, Oct. 10, 1978”, and paragraph 0022 of JP2015-157893A can be suitably used. In addition, in the process for dispersing the pigment, a refining treatment of particles in a salt milling step may be performed. With regard to the materials, equipment, treatment conditions, and the like used in the salt milling step, reference can be made to, for example, the description in JP2015-194521A and JP2012-046629A.

It is preferable that, in the preparation of the photosensitive coloring composition, the photosensitive coloring composition is filtered through a filter for the purpose of removing foreign matters, reducing defects, or the like. As the filter, any filters that have been used in the related art for filtration use and the like may be used without particular limitation. Examples of a material of the filter include: a fluororesin such as polytetrafluoroethylene (PTFE); a polyamide resin such as nylon (for example, nylon-6 or nylon-6,6); and a polyolefin resin (including a polyolefin resin having a high density and an ultrahigh molecular weight) such as polyethylene or polypropylene (PP). Among these materials, polypropylene (including a high-density polypropylene) and nylon are preferable.

The pore size of the filter is preferably 0.01 μm to 7.0 μm, more preferably 0.01 μm to 3.0 μm, and still more preferably 0.05 μm to 0.5 μm. In a case where the pore size of the filter is within the above-described range, fine foreign matters can be reliably removed. With regard to the pore size value of the filter, reference can be made to a nominal value of filter manufacturers. As the filter, various filters provided by Nihon Pall Corporation (DFA4201NIEY and the like), Toyo Roshi Kaisha., Ltd., Nihon Entegris K.K. (formerly Nippon Microlith Co., Ltd.), Kitz Micro Filter Corporation, and the like can be used.

In addition, it is preferable that a fibrous filter material is used as the filter. Examples of the fibrous filter material include a polypropylene fiber, a nylon fiber, and a glass fiber. Examples of a commercially available product include SBP type series (SBP008 and the like), TPR type series (TPR002, TPR005, and the like), or SHPX type series (SHPX003 and the like), all manufactured by Roki Techno Co., Ltd.

In a case of using a filter, different filters (for example, a first filter, a second filter, and the like) may be combined. In this case, the filtration with each of the filters may be performed once or may be performed twice or more times. In addition, filters having different pore sizes within the above-described range may be combined. In addition, the filtration through the first filter may be performed with only a dispersion liquid, the other components may be mixed therewith, and then the filtration through the second filter may be performed.

(Cured Substance)

The cured substance according to the embodiment of the present disclosure is a cured substance obtained by curing the photosensitive coloring composition according to the embodiment of the present disclosure. The cured substance according to the embodiment of the present disclosure can be suitably used in a color filter or the like. Specifically, the cured film according to the embodiment of the present invention can be preferably used as a colored layer (pixel) of a color filter, and more specifically, the cured film according to the embodiment of the present invention can be preferably used as a red-colored layer (red pixel) of a color filter.

The cured substance according to the embodiment of the present disclosure is preferably a film-like cured substance, and the film thickness thereof can be appropriately adjusted depending on the purposes. For example, a film thickness is preferably 20 μm or less, more preferably 10 μm or less, and still more preferably 5 μm or less. The lower limit of the film thickness is preferably 0.1 μm or more, more preferably 0.2 μm or more, and still more preferably 0.3 μm or more.

(Color Filter)

Next, a color filter according to an embodiment of the present disclosure will be described. The color filter according to the embodiment of the present disclosure includes the above-described cured substance according to the embodiment of the present disclosure. More preferably, the color filter according to the embodiment of the present disclosure has a cured film according to the present disclosure as a pixel of the color filter. The color filter according to the embodiment of the present disclosure can be used for a solid-state imaging element such as a charge coupled device (CCD) and a complementary metal-oxide semiconductor (CMOS), an image display device, or the like.

In the color filter according to the embodiment of the present disclosure, the thickness of a film according to the embodiment of the present disclosure can be appropriately adjusted depending on the purposes. The film thickness is preferably 20 μm or less, more preferably 10 μm or less, and still more preferably 5 μm or less. The lower limit of the film thickness is preferably 0.1 μm or more, more preferably 0.2 μm or more, and still more preferably 0.3 μm or more.

In the color filter according to the embodiment of the present disclosure, the width of the pixel is preferably 0.5 μm to 20.0 μm. The lower limit is more preferably 1.0 μm or more and particularly preferably 2.0 μm or more. The upper limit is more preferably 15.0 μm or less and particularly preferably 10.0 μm or less. In addition, the Young's modulus of the pixel is preferably 0.5 GPa to 20 GPa and more preferably 2.5 GPa to 15 GPa.

Each pixel included in the color filter according to the embodiment of the present disclosure preferably has high flatness. Specifically, the surface roughness Ra of the pixel is preferably 100 nm or less, more preferably 40 nm or less, and still more preferably 15 nm or less. The lower limit is not specified, but is preferably, for example, 0.1 nm or more. The surface roughness of the pixel can be measured, for example, using an atomic force microscope (AFM) Dimension 3100 manufactured by Veeco Instruments, Inc. In addition, the contact angle of water on the pixel can be appropriately set to a preferred value and is typically in the range of 500 to 110°. The contact angle can be measured, for example, using a contact angle meter CV-DT-A Model (manufactured by Kyowa Interface Science Co., Ltd.). In addition, it is preferable that the volume resistivity value of the pixel is high. Specifically, the volume resistivity value of the pixel is preferably 10⁹ Ω·cm or more and more preferably 10¹¹ Ω·cm or more. The upper limit is not specified, but is, for example, preferably 10¹⁴ Ω·cm or less. The volume resistivity value of the pixel can be measured, for example, using an ultra-high resistance meter 5410 (manufactured by Advantest Corporation).

In addition, in the color filter according to the embodiment of the present disclosure, a protective layer may be provided on a surface of the film according to the present disclosure. By providing the protective layer, various functions such as oxygen shielding, low reflection, hydrophilicity/hydrophobicity, and shielding of light (ultraviolet rays, near infrared rays, and the like) having a specific wavelength can be imparted. A thickness of the protective layer is preferably 0.01 μm to 10 μm and more preferably 0.1 Vim to 5 μm. Examples of a method for forming the protective layer include a method of forming the protective layer by applying a resin composition dissolved in an organic solvent, a chemical vapor deposition method, and a method of attaching a molded resin with an adhesive material. Examples of components constituting the protective layer include a (meth)acrylic resin, an ene-thiol resin, a polycarbonate resin, a polyether resin, a polyarylate resin, a polysulfone resin, a polyethersulfone resin, a polyphenylene resin, a polyarylene ether phosphine oxide resin, a polyimide resin, a polyamidoimide resin, a polyolefin resin, a cyclic olefin resin, a polyester resin, a styrene resin, a polyol resin, a polyvinylidene chloride resin, a melamine resin, a urethane resin, an aramid resin, a polyamide resin, an alkyd resin, an epoxy resin, a modified silicone resin, a fluororesin, a polyacrylonitrile resin, a cellulose resin, Si, C, W, Al₂O₃, Mo, SiO₂, and Si₂N₄, and two or more kinds of these components may be contained. For example, in a case of a protective layer for oxygen shielding, it is preferable that the protective layer contains a polyol resin, SiO₂, and Si₂N₄. In addition, in a case of a protective layer for low reflection, it is preferable that the protective layer contains a (meth)acrylic resin and a fluororesin.

In a case of forming the protective layer by applying a resin composition, as a method for applying the resin composition, a known method such as a spin coating method, a casting method, a screen printing method, and an ink jet method can be used. As the organic solvent included in the resin composition, a known organic solvent (for example, propylene glycol 1-monomethyl ether 2-acetate, cyclopentanone, ethyl lactate, and the like) can be used. In a case of forming the protective layer by a chemical vapor deposition method, as the chemical vapor deposition method, a known chemical vapor deposition method (thermochemical vapor deposition method, plasma chemical vapor deposition method, and photochemical vapor deposition method) can be used.

The protective layer may contain, as desired, an additive such as organic particles, inorganic particles, an absorber of light (for example, ultraviolet rays, near infrared rays, and the like) having a specific wavelength, a refractive index adjusting agent, an antioxidant, an adhesive agent, and a surfactant. Examples of the organic particles and inorganic particles include polymer particles (for example, silicone resin particles, polystyrene particles, and melamine resin particles), titanium oxide, zinc oxide, zirconium oxide, indium oxide, aluminum oxide, titanium nitride, titanium oxynitride, magnesium fluoride, hollow silica, silica, calcium carbonate, and barium sulfate. As the absorber of light having a specific wavelength, a known absorber can be used. The content of these additives can be appropriately adjusted, but is preferably 0.1% by mass to 70% by mass and more preferably 1% by mass to 60% by mass with respect to the total mass of the protective layer.

In addition, as the protective layer, the protective layers described in paragraphs 0073 to 0092 of JP2017-151176A can also be used.

The color filter may have a base layer. The base layer can be formed, for example, of a composition obtained by removing the colorant from the above-described coloring composition according to the embodiment of the present disclosure. A surface contact angle of the base layer is preferably 200 to 700 in a case of being measured with diiodomethane. In addition, the surface contact angle of the base layer is preferably 300 to 800 in a case of being measured with water. In a case where the surface contact angle of the base layer is within the above-described range, coating property of the resin composition is good. The surface contact angle of the base layer can be adjusted by, for example, adding a surfactant.

<Method for Manufacturing Color Filter>

Next, a method for manufacturing a color filter using the photosensitive coloring composition according to the embodiment of the present disclosure will be described. The color filter can be manufactured through a step of forming a photosensitive coloring composition layer on a support using the above-described photosensitive coloring composition according to the embodiment of the present disclosure, and a step of forming a pattern on the photosensitive coloring composition layer by a photolithography method or a dry etching method. Since, in the photosensitive coloring composition according to the embodiment of the present disclosure, generation of development residue can be suppressed, the present disclosure is particularly effective in a case of manufacturing a color filter in which a pattern is formed on the photosensitive coloring composition layer by a photolithography method.

—Photolithography Method—

First, a case of forming a pattern by a photolithography method to manufacture a color filter will be described. This manufacturing method preferably includes a step of forming a photosensitive coloring composition layer on a support using the photosensitive coloring composition according to the embodiment of the present disclosure, a step of exposing the photosensitive coloring composition layer in a patterned manner, and a step of removing a non-exposed portion of the photosensitive coloring composition layer by development to form a pattern (pixel). A step (pre-baking step) of baking the photosensitive coloring composition layer and a step (post-baking step) of baking the developed pattern (pixel) may be provided, optionally.

In the step of forming a photosensitive coloring composition layer, the photosensitive coloring composition layer is formed on a support using the photosensitive coloring composition according to the embodiment of the present disclosure. The support is not particularly limited, and can be appropriately selected depending on applications. Examples thereof include a glass substrate and a silicon substrate, and a silicon substrate is preferable. In addition, a charge coupled device (CCD), a complementary metal-oxide semiconductor (CMOS), a transparent conductive film, or the like may be formed on the silicon substrate. In some cases, a black matrix for isolating each pixel is formed on the silicon substrate. In addition, a base layer may be provided on the silicon substrate so as to improve adhesiveness to an upper layer, prevent the diffusion of materials, or planarize the surface of the substrate. The base layer may be formed of a composition obtained by removing a colorant from the photosensitive coloring composition described in the present specification, a composition including the resin, polymerizable compound, surfactant, and the like described in the present specification, or the like.

As a method of applying the photosensitive coloring composition, a known method can be used. Examples thereof include a dropping method (drop casting); a slit coating method; a spray method; a roll coating method; a spin coating method (spin coating); a cast coating method; a slit and spin method; a pre-wet method (for example, a method described in JP2009-145395A), various printing methods such as an ink jet (for example, on-demand type, piezo type, thermal type), a discharge printing such as nozzle jet, a flexo printing, a screen printing, a gravure printing, a reverse offset printing, and a metal mask printing; a transfer method using molds and the like; and a nanoimprinting method. A method for applying the ink jet is not particularly limited, and examples thereof include a method described in “Extension of Use of Ink Jet—Infinite Possibilities in Patent—” (February, 2005, S. B. Research Co., Ltd.) (particularly pp. 115 to 133) and methods described in JP2003-262716A, JP2003-185831A, JP2003-261827A, JP2012-126830A, and JP2006-169325A. In addition, with regard to the method for applying the photosensitive coloring composition, reference can be made to the description in WO2017/030174A and WO2017/018419A, the contents of which are incorporated herein by reference.

The photosensitive coloring composition layer formed on the support may be dried (pre-baked). In a case of producing a film by a low-temperature process, pre-baking may not be performed. In a case of performing the pre-baking, the pre-baking temperature is preferably 150° C. or lower, more preferably 120° C. or lower, and still more preferably 110° C. or lower. The lower limit may be set to, for example, 50° C. or higher, or to 80° C. or higher. The pre-baking time is preferably 10 seconds to 300 seconds, more preferably 40 seconds to 250 seconds, and still more preferably 80 seconds to 220 seconds. The pre-baking can be performed using a hot plate, an oven, or the like.

<<Exposing Step>>

Next, the photosensitive coloring composition layer is exposed in a patterned manner (exposing step). For example, the photosensitive coloring composition layer can be exposed in a patterned manner using a stepper exposure device or a scanner exposure device through a mask having a predetermined mask pattern. Thus, the exposed portion can be cured.

Examples of the radiation (light) which can be used during the exposure include g-rays and i-rays. In addition, light (preferably light having a wavelength of 180 nm to 300 nm) having a wavelength of 300 nm or less can be used. Examples of the light having a wavelength of 300 nm or less include KrF-rays (wavelength: 248 nm) and ArF-rays (wavelength: 193 nm), and KrF-rays (wavelength: 248 nm) are preferable. In addition, a long-wave light source of 300 nm or more can be used.

In addition, in a case of exposure, the photosensitive composition layer may be irradiated with light continuously to expose the photosensitive composition layer, or the photosensitive composition layer may be irradiated with light in a pulse to expose the photosensitive composition layer (pulse exposure). The pulse exposure refers to an exposing method in which light irradiation and resting are repeatedly performed in a short cycle (for example, millisecond-level or less).

For example, the irradiation amount (exposure amount) is, for example, preferably 0.03 J/cm² to 2.5 J/cm² and more preferably 0.05 J/cm² to 1.0 J/cm². The oxygen concentration during the exposure can be appropriately selected, and the exposure may also be performed, for example, in a low-oxygen atmosphere having an oxygen concentration of 19% by volume or less (for example, 15% by volume, 5% by volume, and substantially oxygen-free) or in a high-oxygen atmosphere having an oxygen concentration of more than 21% by volume (for example, 22% by volume, 30% by volume, and 50% by volume), in addition to an atmospheric air. In addition, the exposure illuminance can be appropriately set, and can be preferably selected from a range of 1,000 W/m² to 100,000 W/m² (for example, 5,000 W/m², 15,000 W/m², or 35,000 W/m²). Appropriate conditions of each of the oxygen concentration and the exposure illuminance may be combined, and for example, a combination of the oxygen concentration of 10% by volume and the illuminance of 10,000 W/m², a combination of the oxygen concentration of 35% by volume and the illuminance of 20,000 W/m², or the like is available.

Next, the non-exposed portion of the photosensitive coloring composition layer is removed by development to form a pattern (pixel). The non-exposed portion of the coloring composition layer can be removed by development using a developer. Thus, the photosensitive coloring composition layer of the non-exposed portion in the exposing step is eluted into the developer, and as a result, only a photocured portion remains. As the developer, an organic alkali developer causing no damage on a base of element, circuit, or the like is preferable. The temperature of the developer is preferably, for example, 20° C. to 30° C. The development time is preferably 20 seconds to 180 seconds. In addition, in order to improve residue removing properties, a step of removing the developer by shaking off per 60 seconds and supplying a fresh developer may be repeated multiple times.

Examples of the developer include an organic solvent and an alkali developer, and an alkali developer is preferably used. As the alkali developer, an alkaline aqueous solution (alkali developer) in which an alkali agent is diluted with pure water is preferable. Examples of the alkali agent include organic alkaline compounds such as ammonia, ethylamine, diethylamine, dimethylethanolamine, diglycol amine, diethanolamine, hydroxyamine, ethylenediamine, tetramethylammonium hydroxide, tetraethylammonium hydroxide, tetrapropylammonium hydroxide, tetrabutylammonium hydroxide, ethyltrimethylammonium hydroxide, benzyltrimethylammonium hydroxide, dimethylbis(2-hydroxyethyl)ammonium hydroxide, choline, pyrrole, piperidine, and 1,8-diazabicyclo[5.4.0]-7-undecene, and inorganic alkaline compounds such as sodium hydroxide, potassium hydroxide, sodium carbonate, sodium hydrogen carbonate, sodium silicate, and sodium metasilicate. In consideration of environmental aspects and safety aspects, the alkali agent is preferably a compound having a high molecular weight. The concentration of the alkali agent in the alkaline aqueous solution is preferably 0.001% by mass to 10% by mass and more preferably 0.01% by mass to 1% by mass. In addition, the developer may further contain a surfactant. Examples of the surfactant include the surfactants described above, and the surfactant is preferably a nonionic surfactant. From the viewpoint of transportation, storage, and the like, the developer may be first produced as a concentrated solution and then diluted to a concentration required upon the use. The dilution ratio is not particularly limited, and can be set to, for example, a range of 1.5 to 100 times. In addition, it is also preferable to wash (rinse) with pure water after development. In addition, it is preferable that the rinsing is performed by supplying a rinsing liquid to the photosensitive coloring composition layer after development while rotating the support on which the photosensitive coloring composition layer after development is formed. In addition, it is preferable that the rinsing is performed by moving a nozzle discharging the rinsing liquid from a center of the support to a peripheral edge of the support. In this case, in the movement of the nozzle from the center of the support to the peripheral edge of the support, the nozzle may be moved while gradually decreasing the moving speed of the nozzle. By performing rinsing in this manner, in-plane variation of rinsing can be suppressed. In addition, the same effect can be obtained by gradually decreasing the rotating speed of the support while moving the nozzle from the center of the support to the peripheral edge of the support.

After the development, it is preferable to carry out an additional exposure treatment or a heating treatment (post-baking) after carrying out drying. The additional exposure treatment or the post-baking is a curing treatment after development in order to complete curing. The heating temperature in the post-baking is preferably, for example, 100° C. to 240° C. and more preferably 200° C. to 240° C. The film after development is post-baked continuously or batchwise using a heating unit such as a hot plate, a convection oven (hot air circulation dryer), and a high-frequency heater under the above-described conditions. In a case of performing the additional exposure treatment, light used for the exposure is preferably light having a wavelength of 400 nm or less. In addition, the additional exposure treatment may be carried out by the method described in KR10-2017-0122130A.

—Dry Etching Method—

Next, a case of forming a pattern by a dry etching method to manufacture a color filter will be described. Pattern formation by a dry etching method preferably includes a step of forming a photosensitive coloring composition layer on a support using the photosensitive coloring composition according to the embodiment of the present disclosure and curing the entire photosensitive coloring composition layer to form a cured composition layer, a step of forming a photoresist layer on the cured composition layer, a step of exposing the photoresist layer in a patterned manner and then developing the photoresist layer to form a resist pattern, and a step of dry-etching the cured composition layer through this resist pattern as a mask and using an etching gas. It is preferable that pre-baking treatment is further performed in order to form the photoresist layer. In particular, as the forming process of the photoresist layer, it is desirable that a heating treatment after exposure and a heating treatment after development (post-baking treatment) are performed. The details of the pattern formation by the dry etching method can be found in paragraphs 0010 to 0067 of JP2013-064993A, the content of which is incorporated herein by reference.

(Solid-State Imaging Element)

It is preferable that the solid-state imaging element according to the embodiment of the present disclosure includes the cured substance according to the embodiment of the present disclosure and has the above-described color filter according to the embodiment of the present disclosure. The configuration of the solid-state imaging element according to the embodiment of the present disclosure is not particularly limited as long as the solid-state imaging element is configured to include the film according to the present disclosure and functions as a solid-state imaging element. Examples of the configuration include the following configurations.

The solid-state imaging element is configured to have a plurality of photodiodes constituting a light receiving area of the solid-state imaging element (a charge coupled device (CCD) image sensor, a complementary metal-oxide semiconductor (CMOS) image sensor, or the like), and a transfer electrode formed of polysilicon or the like on a substrate; have a light-shielding film having openings only over the light receiving section of the photodiodes on the photodiodes and the transfer electrodes; have a device-protective film formed of silicon nitride or the like, which is formed to coat the entire surface of the light-shielding film and the light receiving section of the photodiodes, on the light-shielding film; and have a color filter on the device-protective film. Further, the solid-state imaging element may also be configured, for example, such that it has a light collecting unit (for example, a microlens, which is the same hereinafter) on a device-protective film under a color filter (a side closer to the substrate), or has a light collecting unit on a color filter. In addition, the color filter may have a structure in which each colored pixel is embedded in a space partitioned in, for example, a lattice shape by a partition wall. In this case, it is preferable that the partition wall has a lower refractive index than each colored pixel. Examples of an imaging device having such a structure include the devices described in JP2012-227478A, JP2014-179577A, WO2018/043654A, and US2018/0040656A. An imaging device including the solid-state imaging element according to the embodiment of the present disclosure can also be used as a vehicle camera or a surveillance camera, in addition to a digital camera or electronic apparatus (mobile phones or the like) having an imaging function.

In addition, in the solid-state imaging element according to the embodiment of the present disclosure, by providing an ultraviolet absorbing layer (UV cut filter) in the structure of the solid-state imaging element, as described in JP2019-211559A, light resistance of the color filter may be improved.

(Image Display Device)

It is preferable that the image display device according to the embodiment of the present disclosure includes the cured substance according to the embodiment of the present disclosure and has the above-described color filter according to the embodiment of the present disclosure. Examples of the image display device include a liquid crystal display device or an organic electroluminescent display device. The definitions of image display devices or the details of the respective image display devices are described in, for example, “Electronic Display Device (Akio Sasaki, Kogyo Chosakai Publishing Co., Ltd., published in 1990)”, “Display Device (Sumiaki Ibuki, Sangyo Tosho Co., Ltd.)”, and the like. In addition, the liquid crystal display device is described in, for example, “Liquid Crystal Display Technology for Next Generation (edited by Tatsuo Uchida, Kogyo Chosakai Publishing Co., Ltd., published in 1994)”. The liquid crystal display device to which the present disclosure can be applied is not particularly limited, and can be applied to, for example, liquid crystal display devices employing various systems described in the “Liquid Crystal Display Technology for Next Generation”.

(Asymmetric Diketopyrrolopyrrole Compound)

An asymmetric diketopyrrolopyrrole compound according to an embodiment of the present disclosure is an asymmetric diketopyrrolopyrrole compound represented by Formula 2, and is preferably an asymmetric diketopyrrolopyrrole compound represented by Formula 3.

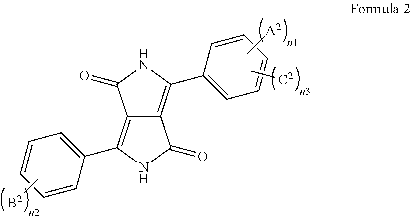

In Formula 2, A²'s each independently represent a monovalent organic group having an acidic functional group or a basic functional group, B²'s each independently represent a monovalent organic group, C²'s each independently represent a monovalent organic group not having an acidic functional group or a basic functional group, n1 represents an integer of 1 to 5, n2 represents an integer of 0 to 5, n3 represents an integer of 0 to 4, and a phenyl group to which A² and C² are bonded and a phenyl group to which B² is bonded are different groups.

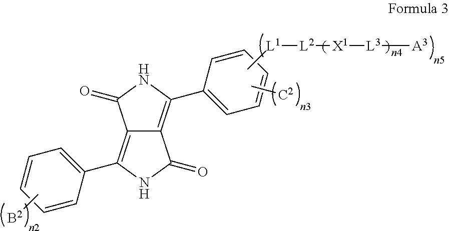

In Formula 3, A³'s each independently represent an acidic functional group or a basic functional group, B²'s each independently represent a monovalent organic group not having an acidic functional group and a basic functional group, C²'s each independently represent a monovalent organic group not having an acidic functional group and a basic functional group, X¹'s each independently represent an ether bond, a thioether bond, a sulfonamide bond, or a urea bond, L¹'s each independently represent a single bond or an ether bond, L²'s and L³'s each independently represent an alkylene group, n2 represents an integer of 0 to 5, n3 represents an integer of 0 to 4, n4's each independently represent 0 or 1, n5 represents an integer of 1 to 5, a group having A³ at a terminal, a phenyl group to which C² is bonded, and a phenyl group to which B² is bonded are different groups, and in a case where L¹ is an ether bond, B² is an electron donating group not having an acidic functional group and a basic functional group and n2 represents an integer of 1 to 5.

Preferred aspects of Formula 2 and Formula 3 in the asymmetric diketopyrrolopyrrole compound according to the embodiment of the present disclosure are the same as the preferred aspects of Formula 2 and Formula 3 in the photosensitive coloring composition according to the embodiment of the present disclosure described above.

### EXAMPLES

Hereinafter, the present disclosure will be described in detail with reference to examples, but the present disclosure is not limited thereto.

In the examples, “%” and “parts” respectively indicate “% by mass” and “parts by mass” unless otherwise specified. In a polymer compound, the molecular weight indicates the weight-average molecular weight (Mw) and the proportion of constitutional units indicates mole percentage unless otherwise specified.

The weight-average molecular weight (Mw) is a value in terms of polystyrene obtained by performing measurement using a gel permeation chromatography (GPC) method.

DPP-1 to 26 used in Examples are the same compounds as DPP-1 to 26 described above, respectively.

### Synthesis Example 1: Synthesis of DPP-1

160 parts of tert-amyl alcohol, 34 parts of a compound A-1, and 54 parts of sodium tert-pentoxide were added to a three-neck flask replaced with nitrogen, and the mixture was heated to 110° C. and stirred. Next, 40 parts of a compound B-1 shown below, which was synthesized by the method described in Tetrahedron, 58 (2002), 5547 to 5565, was added thereto. After reacting at 120° C. for 4 hours, the mixture was cooled to 70° C., and then filtered and washed with methanol by adding 320 parts of methanol and 400 parts of water, thereby obtaining 39 parts of an asymmetric diketopyrrolopyrrole compound DPP-1.

### Synthesis Examples 2 to 20, 22, 23, 25, and 26: Synthesis of DPP-2 to 20, 22, 23, 25, and 26

DPP-2 to 20, 22, and 23 were produced in the same manner as in Synthesis Example 1 described above, except that the compound A-1 and the compound B-1, which were the raw materials, were changed as shown in Table 1.

### Synthesis Example 21: Synthesis of DPP-21

After synthesizing by the same method as in Synthesis Example 1 described above, 10 parts of the DPP precursor was added to 100 parts of acetic acid. After adding 15 parts of a 25% by mass hydrogen bromide-acetic acid solution, the mixture was heated to 50° C. and stirred for 3 hours. The mixture was cooled to 25° C., and then filtered and washed with acetonitrile to obtain 6 parts of an asymmetric diketopyrrolopyrrole compound DPP-21.

### Synthesis Example 24: Synthesis of DPP-24

After synthesizing by the same method as in Synthesis Example 1 described above, 10 parts of the DPP precursor, 15 parts of potassium carbonate, and 15 parts of 1-bromohexane were added to 100 parts of NMP and 50 parts of DMF. After reacting at 120° C. for 12 hours, the mixture was cooled to 50° C., and then filtered by adding 100 parts of methanol and 300 parts of water. Further, the obtained filtrate was added to 100 parts of acetic acid, and after adding 15 parts of a 25% by mass hydrogen bromide-acetic acid solution, the mixture was heated to 50° C. and stirred for 3 hours. The mixture was cooled to 25° C., and then filtered and washed with acetonitrile to obtain 3 parts of an asymmetric diketopyrrolopyrrole compound DPP-24.

In addition, ¹H-NMR measurement data for some of the obtained diketopyrrolopyrrole compound are shown below.

DPP-1: ¹H-NMR (DMSO-d6) δ (ppm)=0.99, 2.40, 2.48, 3.60, 7.39, 7.50, 11.24

DPP-2: ¹H-NMR (DMSO-d6) δ (ppm)=0.99, 2.48, 3.61, 7.51, 7.67, 8.42, 8.48, 11.34

DPP-3: ¹H-NMR (DMSO-d6) δ (ppm)=1.00, 2.48, 3.61, 7.51, 7.57, 8.43, 8.48, 11.29

DPP-4: ¹H-NMR (DMSO-d6) δ (ppm)=2.17, 2.40, 3.47, 7.39, 7.47, 8.38, 8.42, 11.24

DPP-6: ¹H-NMR (DMSO-d6) δ (ppm)=2.17, 2.40, 2.47, 3.55, 4.56, 7.39, 7.50, 8.39, 8.44, 11.26

DPP-10: ¹H-NMR (DMSO-d6) δ (ppm)=0.90, 1.52, 2.40, 2.94, 4.43, 7.18, 7.40, 7.55, 8.39, 8.45, 11.28

DPP-11: ¹H-NMR (DMSO-d6) δ (ppm)=1.88, 2.16, 2.37, 2.39, 4.12, 7.12, 7.37, 8.35, 8.47, 11.17

DPP-23: ¹H-NMR (DMSO-d6) δ (ppm)=1.87, 2.15, 2.36, 3.86, 4.11, 7.11, 7.13, 8.44, 8.46, 11.14

TABLE 1

| Asymmetric DPP | Raw material A group | Raw material B group |
|---|---|---|
| DPP-1 | A-1 | B-1 |
| DPP-2 | A-1 | B-2 |
| DPP-3 | A-1 | B-2 |
| DPP-4 | A-2 | B-1 |
| DPP-5 | A-2 | B-3 |
| DPP-6 | A-3 | B-1 |
| DPP-7 | A-4 | B-1 |
| DPP-8 | A-5 | B-1 |
| DPP-9 | A-6 | B-1 |
| DPP-10 | A-7 | B-1 |
| DPP-11 | A-8 | B-1 |
| DPP-12 | A-8 | B-3 |
| DPP-13 | A-9 | B-1 |
| DPP-14 | A-10 | B-1 |
| DPP-15 | A-11 | B-1 |
| DPP-16 | A-12 | B-1 |
| DPP-17 | A-13 | B-1 |
| DPP-18 | A-14 | B-1 |
| DPP-19 | A-15 | B-1 |
| DPP-20 | A-16 | B-1 |
| DPP-21 | A-17 | B-1 |
| DPP-22 | A-1 | B-4 |
| DPP-23 | A-8 | B-4 |
| DPP-24 | A-17 | B-1 |
| DPP-25 | A-11 | B-5 |
| DPP-26 | A-18 | B-1 |

Compounds A-1 to A-18 and compounds B-1 to B-5 described in Table 1 are shown below.

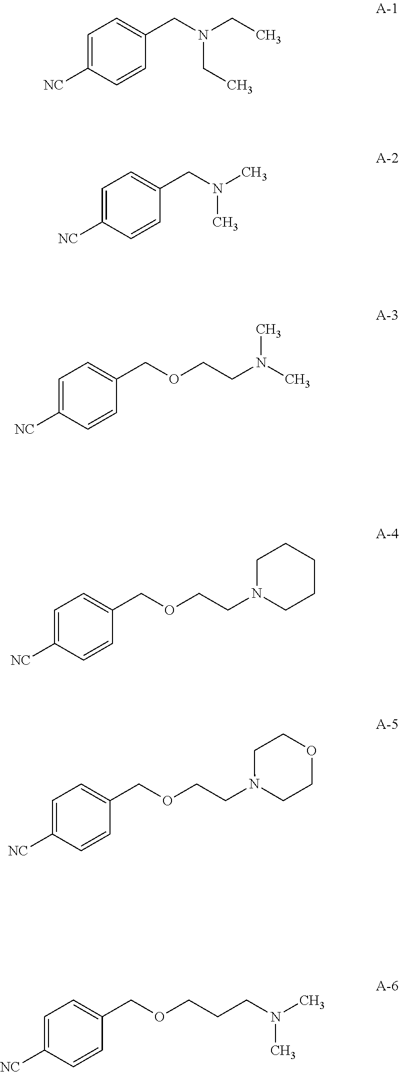 [SMILES: CCN(CC)Cc1ccc(C#N)cc1 ; CN(C)Cc1ccc(C#N)cc1 ; CN(C)CCOCc1ccc(C#N)cc1 ; N#Cc1ccc(COCCN2CCCCC2)cc1 ; N#Cc1ccc(COCCN2CCOCC2)cc1 ; CN(C)CCCOCc1ccc(C#N)cc1]

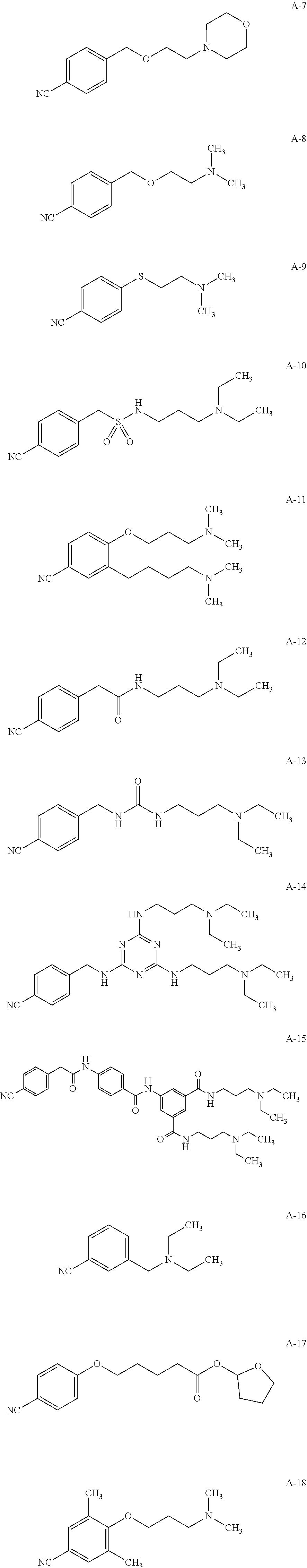

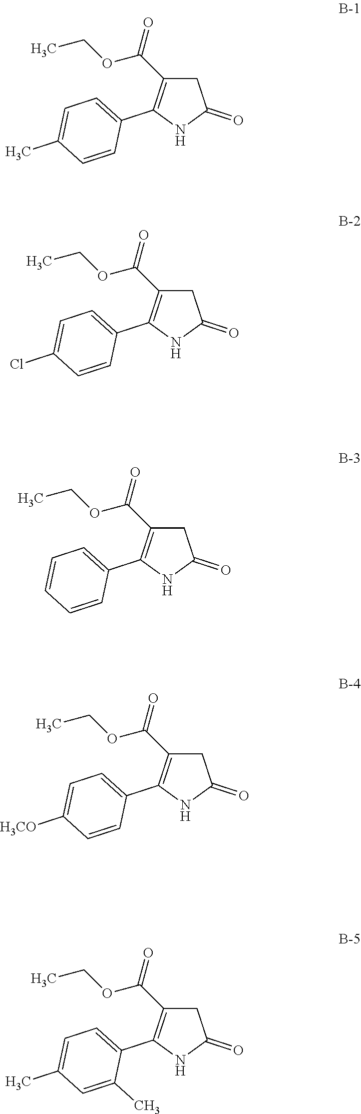 [SMILES: CCOC(=O)C1=C(c2ccc(C)cc2)NC(=O)C1 ; CCOC(=O)C1=C(c2ccc(Cl)cc2)NC(=O)C1 ; CCOC(=O)C1=C(c2ccccc2)NC(=O)C1 ; CCOC(=O)C1=C(c2ccc(OC)cc2)NC(=O)C1 ; CCOC(=O)C1=C(c2ccc(C)cc2C)NC(=O)C1]

<Preparation of Pigment Composition>

A mixed solution in which the resin, pigment, diketopyrrolopyrrole compound (DPP), solvent, and other components shown in Table 2 or Table 3 were mixed at the ratios shown in Table 2 or Table 3 was mixed and dispersed for 3 hours using a beads mill (zirconia beads having a diameter of 0.3 mm). Next, using a high-pressure disperser NANO-3000-10 (manufactured by Nippon BEE Chemical Co., Ltd.) equipped with a pressure reducing mechanism, the pigment dispersion liquid was further dispersed under a pressure of 2,000 kg/cm² at a flow rate of 500 g/min. This dispersion treatment was repeated 10 times to obtain a pigment composition.

TABLE 2

| | | | | | | | | | | | | | | | | | | | Total | | Con- | | |
|---|---|---|---|---|---|---|---|---|---|---|---|---|---|---|---|---|---|---|---|---|---|---|---|
| | | | | | | | | | | | | | | | | Con- | | | content | Con- | tent of | | |
| | | | | | | | | | | | | | | | | tent | Ratio | Ratio | of DPP + | tent of | pig- | | |
| | | | | | | | | | | | | | | | | of | of | of | pig- | pig- | ment | | |
| | | | | | | | | | | | | | | | | resin/ | red | yellow | ment in | ment | in pig- | | |
| | | | | | | | | | | | | | | | | con- | pig- | pig- | pigment | in total | ment | | |
| | DPP (ratio | | | | | | | | | | | | | | Solid | tent | ment | ment | com- | solid | com- | | |
| Pigment | listed is | Part | Pig- | Part | Pig- | Part | Pig- | Part | | Part | | Part | Other | Part | content | of | and | and | position | content | position | m^{A}/ | |
| com- | molar | by | ment | by | ment | by | ment | by | Resin | by | | by | com- | by | (% by | pig- | de- | de- | (% by | (% by | (% by | m^{A} + | MP/ |
| position | ratio) | mass | 1 | mass | 2 | mass | 3 | mass | 1 | mass | Solvent | mass | ponents | mass | mass) | ment | rivative | rivative | mass) | mass) | mass) | (m^{B}) | MA |
| R-1 | DPP-1 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-2 | DPP-2 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-3 | DPP-3 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-4 | DPP-4 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-5 | DPP-5 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-6 | DPP-6 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-7 | DPP-7 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-8 | DPP-8 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-9 | DPP-9 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-10 | DPP-10 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-11 | DPP-11 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-12 | DPP-12 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-13 | DPP-13 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-14 | DPP-14 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-15 | DPP-15 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-16 | DPP-16 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-17 | DPP-17 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-18 | DPP-18 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-19 | DPP-19 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-20 | DPP-20 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-21 | DPP-21 | 4.4 | PR272 | 18.3 | PR254 | 20.9 | PY139 | 21.0 | PA-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-22 | DPP-22 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-23 | DPP-23 | 4.4 | PR272 | 18.3 | PR254 | 20.9 | PV139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-24 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 95 | 13.8 |
| | C2 = 95:5 | | | | | | | | | | | | | | | | | | | | | | |
| R-25 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 90 | 13.8 |
| | C2 = 90:10 | | | | | | | | | | | | | | | | | | | | | | |
| R-26 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 80 | 13.8 |
| | C2 = 80:20 | | | | | | | | | | | | | | | | | | | | | | |
| R-27 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 50 | 13.8 |
| | C2 = 50:50 | | | | | | | | | | | | | | | | | | | | | | |
| R-28 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| | 20 = 95:5 | | | | | | | | | | | | | | | | | | | | | | |
| R-29 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| | 20 = 90:10 | | | | | | | | | | | | | | | | | | | | | | |
| R-30 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| | 20 = 85:15 | | | | | | | | | | | | | | | | | | | | | | |
| R-31 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| | 20 = 80:20 | | | | | | | | | | | | | | | | | | | | | | |
| R-32 | DPP-11 | 4.4 | PR272 | 39.2 | — | 0.0 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-33 | DPP-11 | 4.4 | PR254 | 39.2 | — | 0.0 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-34 | DPP-11 | 4.4 | PO71 | 39.2 | — | 0.0 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |

TABLE 3

| | | | | | | | | | | | | | | | | | | | Total | | Con- | | |
|---|---|---|---|---|---|---|---|---|---|---|---|---|---|---|---|---|---|---|---|---|---|---|---|
| | | | | | | | | | | | | | | | | Con- | | | content | Content | tent of | | |
| | | | | | | | | | | | | | | | | tent | | Ratio | of DPP + | of | pig- | | |
| | | | | | | | | | | | | | | | | of | Ratio | of | pigment | pig- | ment in | | |
| | | | | | | | | | | | | | | | | resin/ | of red | yellow | in pig- | ment in | pig- | | |
| | DPP | | | | | | | | | | | | | | | con- | pig- | pig- | ment | total | ment | | |
| Pig- | (ratio | | | | | | | | | | | | | | Solid | tent | ment | ment | com- | solid | com- | | |
| ment | listed | Part | Pig- | Part | Pig- | Part | Pig- | Part | | Part | | Part | Other | Part | content | of | and | and | position | content | position | m^{A}/ | |
| com- | is molar | by | ment | by | ment | by | ment | by | Resin | by | | by | com- | by | (% by | pig- | de- | de- | % by | (% by | (% by | (m^{A} + | MP/ |
| position | ratio) | mass | 1 | mass | 2 | mass | 3 | mass | 1 | mass | Solvent | mass | ponents | mass | mass) | ment | rivative | rivative | (mass) | mass) | mass) | m^{B}) | MA |
| R-35 | DPP-11 | 4.4 | PR272 | 18.3 | PR254 | 20.9 | PY185 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-36 | DPP-11 | 4.4 | PR272 | 18.3 | PR254 | 20.9 | PY150 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-37 | DPP-11 | 4.4 | PR272 | 18.3 | PR254 | 20.9 | PY138 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-38 | DPP-11 | 13.1 | PR272 | 18.3 | PR254 | 20.9 | PY139 | 21.0 | PB-1 | 88.9 | PGMEA | 416.5 | SY-1 | 2.9 | 17.7 | 0.35 | 3.0 | 7.1 | 13.1 | 74.1 | 10.4 | 100 | 4.6 |
| R-39 | DPP-11 | 32.7 | PR272 | 18.3 | PR254 | 20.9 | PY139 | 21.0 | PB-1 | 111.8 | PGMEA | 523.6 | SY-1 | 2.9 | 17.7 | 0.35 | 1.2 | 7.1 | 13.1 | 74.1 | 8.2 | 100 | 1.8 |
| R-40 | DPP-11 | 49.0 | PR272 | 18.3 | PR254 | 20.9 | PY139 | 21.0 | PB-1 | 130.8 | PGMEA | 612.9 | SY-1 | 2.9 | 17.7 | 0.35 | 0.80 | 7.1 | 13.1 | 74.1 | 7.0 | 100 | 1.2 |
| R-41 | DPP-11 | 3.9 | PR272 | 18.3 | PR254 | 20.9 | PY139 | 21.0 | PB-1 | 78.2 | PGMEA | 366.5 | SY-1 | 2.9 | 17.7 | 0.35 | 10.0 | 7.1 | 13.1 | 74.1 | 11.8 | 100 | 15.4 |
| R-42 | DPP-11 | 3.6 | PR272 | 18.3 | PR254 | 20.9 | PY139 | 21.0 | PB-1 | 77.8 | PGMEA | 364.6 | SY-1 | 2.9 | 17.7 | 0.35 | 11.0 | 7.1 | 13.1 | 74.1 | 11.8 | 100 | 16.9 |
| R-43 | DPP-11 | 3.3 | PR272 | 18.3 | PR254 | 20.9 | PY139 | 21.0 | PB-1 | 77.5 | PGMEA | 362.9 | SY-1 | 2.9 | 17.7 | 0.35 | 12.0 | 7.1 | 13.1 | 74.1 | 11.9 | 100 | 18.4 |
| R-44 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| | 6 = 50:50 | | | | | | | | | | | | | | | | | | | | | | |
| R-45 | DPP-11 | 4.4 | PR272 | 18.3 | PR254 | 20.9 | PY139 | 21.0 | PB-2 | 78.8 | PGMEA | 368.9 | SY-1 | 2.9 | 17.7 | 0.35 | 9.0 | 7.1 | 13.1 | 74.1 | 11.7 | 100 | 13.8 |
| R-46 | DPP-11 | 4.4 | PR272 | 18.3 | PR254 | 20.9 | PY139 | 21.0 | PB-3 | 78.8 | PGMEA | 368.9 | SY-1 | 2.9 | 17.7 | 0.35 | 9.0 | 7.1 | 13.1 | 74.1 | 11.7 | 100 | 13.8 |
| R-47 | DPP-11 | 4.4 | PR272 | 18.3 | PR254 | 20.9 | PY139 | 21.0 | PB-4 | 78.8 | PGMEA | 368.9 | SY-1 | 2.9 | 17.7 | 0.35 | 9.0 | 7.1 | 13.1 | 74.1 | 11.7 | 100 | 13.8 |
| R-48 | DPP-11 | 4.4 | PR272 | 18.3 | PR254 | 20.9 | PY139 | 21.0 | PB-5 | 78.8 | PGMEA | 368.9 | SY-1 | 2.9 | 17.7 | 0.35 | 9.0 | 7.1 | 13.1 | 74.1 | 11.7 | 100 | 13.8 |
| R-49 | DPP-1 | 7.3 | PR272 | 18.3 | PR254 | 20.9 | PY139 | 21.0 | PB-1 | 78.7 | PGMEA | 368.6 | — | 0.0 | 17.7 | 0.35 | 5.4 | — | 13.1 | 74.1 | 11.7 | 100 | 8.3 |
| R-50 | DPP-6 | 7.3 | PR272 | 18.3 | PR254 | 20.9 | PY139 | 21.0 | PB-1 | 78.7 | PGMEA | 368.6 | — | 0.0 | 17.7 | 0.35 | 5.4 | — | 13.1 | 74.1 | 11.7 | 100 | 8.3 |
| R-51 | DPP-11 | 7.3 | PR272 | 18.3 | PR254 | 20.9 | PY139 | 21.0 | PB-1 | 78.7 | PGMEA | 368.6 | — | 0.0 | 17.7 | 0.35 | 5.4 | — | 13.1 | 74.1 | 11.7 | 100 | 8.3 |
| R-52 | DPP-22 | 7.3 | PR272 | 18.3 | PR254 | 20.9 | PY139 | 21.0 | PB-1 | 78.7 | PGMEA | 368.6 | — | 0.0 | 17.7 | 0.35 | 5.4 | — | 13.1 | 74.1 | 11.7 | 100 | 8.3 |
| R-53 | DPP-23 | 7.3 | PR272 | 18.3 | PR254 | 20.9 | PY139 | 21.0 | PB-1 | 78.7 | PGMEA | 368.6 | — | 0.0 | 17.7 | 0.35 | 5.4 | — | 13.1 | 74.1 | 11.7 | 100 | 8.3 |
| R-54 | DPP-11 | 6.6 | PR272 | 28.0 | PR254 | 31.0 | — | 0.0 | PB-1 | 76.5 | PGMEA | 358.2 | — | 0.0 | 17.7 | 0.35 | 8.9 | — | 13.1 | 74.1 | 11.8 | 100 | 9.0 |
| R-55 | DPP-11 | 4.4 | — | 0.0 | — | 0.0 | PY139 | 60.0 | PB-1 | 85.0 | PGMEA | 397.9 | SY-1 | 8.4 | 17.7 | 0.35 | — | 7.1 | 13.1 | 74.1 | 10.8 | 100 | 13.6 |
| R-56 | DPP-11 | 4.4 | PR272 | 7.8 | PR254 | 31.4 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-57 | DPP-11 | 4.4 | PR272 | 28.8 | PR254 | 10.4 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-58 | DPP-11 | 4.4 | PR272 | 23.3 | PR254 | 25.9 | PY139 | 11.0 | PB-1 | 77.2 | PGMEA | 361.5 | SY-1 | 1.5 | 17.7 | 0.35 | 9.0 | 7.1 | 13.1 | 74.1 | 11.9 | 100 | 13.7 |
| R-59 | DPP-11 | 4.4 | PR272 | 13.3 | PR254 | 15.9 | PY139 | 31.0 | PB-1 | 80.4 | PGMEA | 376.8 | SY-1 | 4.4 | 17.7 | 0.35 | 9.0 | 7.1 | 13.1 | 74.1 | 11.4 | 100 | 13.7 |
| R-60 | DPP-1:DPP- | 4.4 | PR272 | 18.3 | PR254 | 20.9 | PY139 | 21.0 | PB-1 | 78.8 | PGMEA | 369.1 | SY-1 | 2.9 | 17.7 | 0.35 | 9.0 | 7.1 | 13.1 | 74.1 | 11.7 | 11 | 13.7 |
| | C2 = 11:89 | | | | | | | | | | | | | | | | | | | | | | |
| R-61 | DPP-24 | 4.4 | PR272 | 18.3 | PR254 | 20.9 | PY139 | 21.0 | PA-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-62 | DPP-25 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-63 | DPP-26 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-64 | DPP-1 | 4.4 | PR272 | 18.3 | PR254 | 20.9 | PY139 | 21.0 | PA-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-65 | DPP-21 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 100 | 13.8 |
| R-C1 | DPP-C1 | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 0 | 13.8 |
| R-C2 | DPP-1:DPP- | 4.4 | PR272 | 18.3 | PR254 | 20.9 | PY139 | 21.0 | PB-1 | 78.8 | PGMEA | 368.9 | SY-1 | 2.9 | 17.7 | 0.35 | 9.0 | 7.1 | 13.1 | 74.1 | 11.7 | 9 | 13.8 |
| | C2 = 9:91 | | | | | | | | | | | | | | | | | | | | | | |

The “ratio of red pigment and derivative” in Tables 2 and 3 indicates the value (mass ratio) of content of the diketopyrrolopyrrole compound/total content of the pigment 1 and the pigment 2, and the “ratio of yellow pigment and derivative” indicates the value (mass ratio) of content of SY-1/content of the pigment 3.

The details of the compounds shown in Tables 2 and 3 are shown below.

PR272: C. I. Pigment Red 272

PR254: C. I. Pigment Red 254

PY139: C. I. Pigment Yellow 139

PY185: C. I. Pigment Yellow 185

PY150: C. I. Pigment Yellow 150

PY138: C. I. Pigment Yellow 138

P071: C. I. Pigment Orange 71

PGMEA: propylene glycol monomethyl ether acetate

SY-1: compound shown below

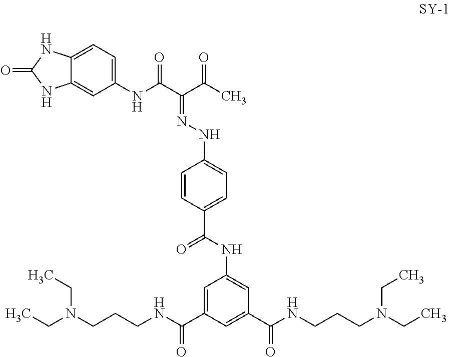 [SMILES: CCN(CC)CCCNC(=O)c1cc(NC(=O)c2ccc(N/N=C(\C(C)=O)C(=O)Nc3ccc4[nH]c(=O)[nH]c4c3)cc2)cc(C(=O)NCCCN(CC)CC)c1]

PB-1: compound shown below, solid content: 30% by mass, PGMEA solution, Mw: 16,000, acid value of solid content: 55 mgKOH/g

PB-2: compound shown below, solid content: 30% by mass, PGMEA solution, Mw: 8,000, acid value of solid content: 53 mgKOH/g

PB-3: compound shown below, solid content: 30% by mass, PGMEA solution, Mw: 15,000, acid value of solid content: 70 mgKOH/g

PB-4: difference product of PB-1 in acid value, acid value of solid content: 40 mgKOH/g

PB-5: difference product of PB-1 in acid value, acid value of solid content: 70 mgKOH/g

PA-1: compound shown below, solid content: 30% by mass, PGMEA solution, Mw: 23,000, acid value of solid content: 30 mgKOH/g

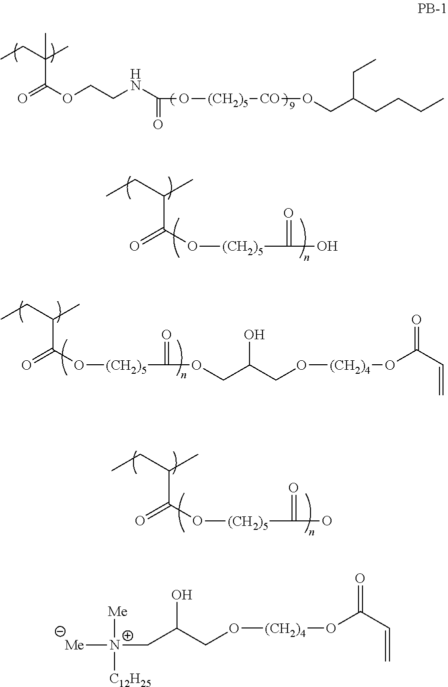

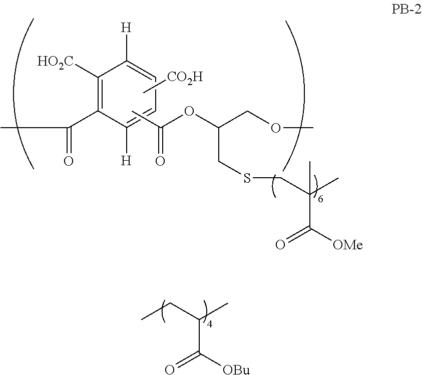

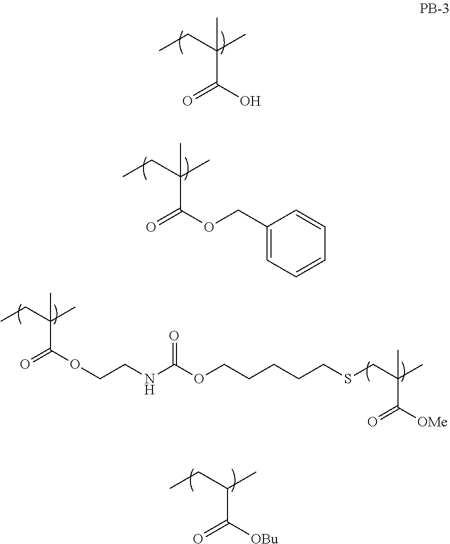

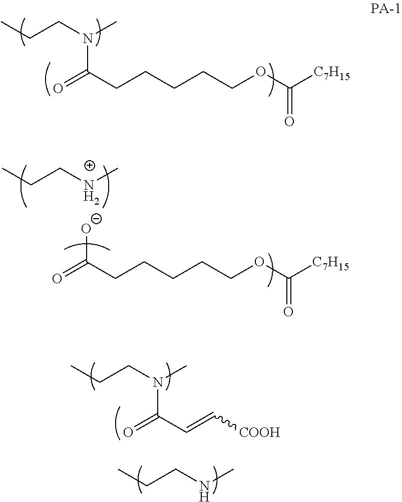

DPP-C1: diketopyrrolopyrrole compound shown below (not including the diketopyrrolopyrrole compound A described above)

DPP-C2: diketopyrrolopyrrole compound shown below

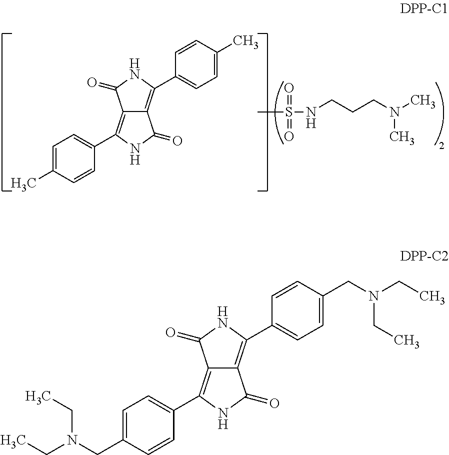

### Examples 1 to 82 and Comparative Examples 1 and 2

<Preparation of Photosensitive Coloring Composition>

The following components were mixed to prepare a photosensitive coloring composition. As for the pigment dispersion liquid, resin, polymerizable compound, photopolymerization initiator, and solvent, the components shown in Table 4 or Table 5 were used.
Pigment composition shown in Table 4 or Table 5: amount shown in Table 4 or Table 5
Resin shown in Table 4 or Table 5: amount shown in Table 4 or Table 5
Polymerizable compound shown in Table 4 or Table 5: amount shown in Table 4 or Table 5
Photopolymerization initiator shown in Table 4 or Table 5: amount shown in Table 4 or Table 5
Surfactant (1% by mass propylene glycol monomethyl ether acetate (PGMEA) solution of the following compound (proportion of a repeating unit means mol %; Mw: 14,000)): 1 part by mass
p-Methoxyphenol: 0.01 parts by mass
Solvent shown in Table 4 or Table 5: amount shown in Table 4 or Table 5

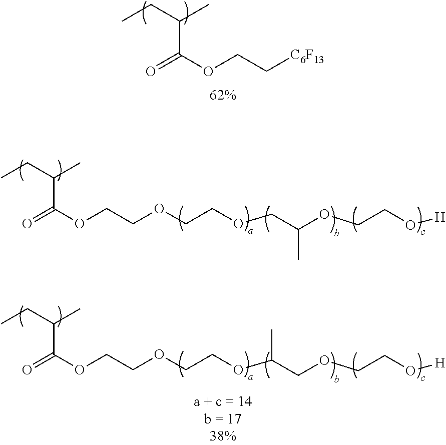

TABLE 4

| | | | | | | | | | | | Content of |
|---|---|---|---|---|---|---|---|---|---|---|---|
| | | | | | | | | | | | pigment in |
| Photosensitive | | | | | | | | | | | total solid |
| coloring | Pigment | Part by | | Part by | Polymerizable | Part by | Photopolymerization | Part by | | Part by | content (% |
| composition | composition | mass | Resin | mass | compound | mass | initiator | mass | Solvent | mass | by mass) |
| RP-1 | R-1 | 300 | PB-6 | 6 | M-1 | 1.8 | I-1 | 3 | S-1 | 100 | 58.8 |
| RP-2 | R-2 | 300 | PB-6 | 6 | M-1 | 1.8 | I-1 | 3 | S-1 | 100 | 58.8 |
| RP-3 | R-3 | 300 | PB-6 | 6 | M-1 | 1.8 | I-1 | 3 | S-1 | 100 | 58.8 |
| RP-4 | R-4 | 300 | PB-6 | 6 | M-1 | 1.8 | I-1 | 3 | S-1 | 100 | 58.8 |
| RP-5 | R-5 | 300 | PB-6 | 6 | M-1 | 1.8 | I-1 | 3 | S-1 | 100 | 58.8 |
| RP-6 | R-6 | 300 | PB-6 | 6 | M-1 | 1.8 | I-1 | 3 | S-1 | 100 | 58.8 |
| RP-7 | R-7 | 300 | PB-6 | 6 | M-1 | 1.8 | I-1 | 3 | S-1 | 100 | 58.8 |
| RP-8 | R-8 | 300 | PB-6 | 6 | M-1 | 1.8 | I-1 | 3 | S-1 | 100 | 58.8 |
| RP-9 | R-9 | 300 | PB-6 | 6 | M-1 | 1.8 | I-1 | 3 | S-1 | 100 | 58.8 |
| RP-10 | R-10 | 300 | PB-6 | 6 | M-1 | 1.8 | I-1 | 3 | S-1 | 100 | 58.8 |
| RP-11 | R-11 | 300 | PB-6 | 6 | M-1 | 1.8 | I-1 | 3 | S-1 | 100 | 58.8 |
| RP-12 | R-12 | 300 | PB-6 | 6 | M-1 | 1.8 | I-1 | 3 | S-1 | 100 | 58.8 |
| RP-13 | R-13 | 300 | PB-6 | 6 | M-1 | 1.8 | I-1 | 3 | S-1 | 100 | 58.8 |
| RP-14 | R-14 | 300 | PB-6 | 6 | M-1 | 1.8 | I-1 | 3 | S-1 | 100 | 58.8 |
| RP-15 | R-15 | 300 | PB-6 | 6 | M-1 | 1.8 | I-1 | 3 | S-1 | 100 | 58.8 |
| RP-16 | R-16 | 300 | PB-6 | 6 | M-1 | 1.8 | I-1 | 3 | S-1 | 100 | 58.8 |
| RP-17 | R-17 | 300 | PB-6 | 6 | M-1 | 1.8 | I-1 | 3 | S-1 | 100 | 58.8 |
| RP-18 | R-18 | 300 | PB-6 | 6 | M-1 | 1.8 | I-1 | 3 | S-1 | 100 | 58.8 |
| RP-19 | R-19 | 300 | PB-6 | 6 | M-1 | 1.8 | I-1 | 3 | S-1 | 100 | 58.8 |
| RP-20 | R-20 | 300 | PB-6 | 6 | M-1 | 1.8 | I-1 | 3 | S-1 | 100 | 58.8 |
| RP-21 | R-21 | 300 | PB-6 | 6 | M-1 | 1.8 | I-1 | 3 | S-1 | 100 | 58.8 |
| RP-22 | R-22 | 300 | PB-6 | 6 | M-1 | 1.8 | I-2 | 3 | S-1 | 100 | 58.8 |
| RP-23 | R-23 | 300 | PB-6 | 6 | M-1 | 1.8 | I-3 | 3 | S-1 | 100 | 58.8 |
| RP-24 | R-24 | 300 | PB-6 | 6 | M-1 | 1.8 | I-1 | 3 | S-1 | 100 | 58.8 |
| RP-25 | R-25 | 300 | PB-6 | 6 | M-1 | 1.8 | I-1 | 3 | S-1 | 100 | 58.8 |
| RP-26 | R-26 | 300 | PB-6 | 6 | M-1 | 1.8 | I-1 | 3 | S-1 | 100 | 58.8 |
| RP-27 | R-27 | 300 | PB-6 | 6 | M-1 | 1.8 | I-1 | 3 | S-1 | 100 | 58.8 |
| RP-28 | R-28 | 300 | PB-6 | 6 | M-1 | 1.8 | I-1 | 3 | S-1 | 100 | 58.8 |
| RP-29 | R-29 | 300 | PB-6 | 6 | M-1 | 1.8 | I-1 | 3 | S-1 | 100 | 58.8 |
| RP-30 | R-30 | 300 | PB-6 | 6 | M-1 | 1.8 | I-1 | 3 | S-1 | 100 | 58.8 |
| RP-31 | R-31 | 300 | PB-6 | 6 | M-1 | 1.8 | I-1 | 3 | S-1 | 100 | 58.8 |
| RP-32 | R-32 | 300 | PB-6 | 6 | M-1 | 1.8 | I-1 | 3 | S-1 | 100 | 58.8 |
| RP-33 | R-33 | 300 | PB-6 | 6 | M-1 | 1.8 | I-1 | 3 | S-1 | 100 | 58.8 |
| RP-34 | R-34 | 300 | PB-6 | 6 | M-1 | 1.8 | I-1 | 3 | S-1 | 100 | 58.8 |
| RP-35 | R-35 | 300 | PB-6 | 6 | M-1 | 1.8 | I-1 | 3 | S-1 | 100 | 58.8 |
| RP-36 | R-36 | 300 | PB-6 | 6 | M-1 | 1.8 | I-1 | 3 | S-1 | 100 | 58.8 |
| RP-37 | R-37 | 300 | PB-6 | 6 | M-1 | 1.8 | I-1 | 3 | S-1 | 100 | 58.8 |
| RP-38 | R-38 | 300 | PB-6 | 6 | M-1 | 1.8 | I-1 | 3 | S-1 | 100 | 52.0 |
| RP-39 | R-39 | 300 | PB-6 | 6 | M-1 | 1.8 | I-1 | 3 | S-1 | 100 | 41.4 |
| RP-40 | R-40 | 300 | PB-6 | 6 | M-1 | 1.8 | I-1 | 3 | S-1 | 100 | 35.4 |
| RP-41 | R-41 | 300 | PB-6 | 6 | M-1 | 1.8 | I-1 | 3 | S-1 | 100 | 59.1 |
| RP-42 | R-42 | 300 | PB-6 | 6 | M-1 | 1.8 | I-1 | 3 | S-1 | 100 | 59.5 |
| RP-43 | R-43 | 300 | PB-6 | 6 | M-1 | 1.8 | I-1 | 3 | S-1 | 100 | 59.7 |
| RP-44 | R-44 | 300 | PB-6 | 6 | M-1 | 1.8 | I-1 | 3 | S-1 | 100 | 58.8 |
| RP-45 | R-45 | 300 | PB-6 | 6 | M-1 | 1.8 | I-1 | 3 | S-1 | 100 | 58.8 |
| RP-46 | R-46 | 300 | PB-6 | 6 | M-1 | 1.8 | I-1 | 3 | S-1 | 100 | 58.8 |
| RP-47 | R-47 | 300 | PB-6 | 6 | M-1 | 1.8 | I-1 | 3 | S-1 | 100 | 58.8 |
| RP-48 | R-48 | 300 | PB-6 | 6 | M-1 | 1.8 | I-1 | 3 | S-1 | 100 | 58.8 |
| RP-49 | R-49 | 300 | PB-6 | 6 | M-1 | 1.8 | I-1 | 3 | S-1 | 100 | 58.8 |
| RP-50 | R-50 | 300 | PB-6 | 6 | M-1 | 1.8 | I-1 | 3 | S-1 | 100 | 58.8 |

TABLE 5

| | | | | | | | | | | | Content of |
|---|---|---|---|---|---|---|---|---|---|---|---|
| | | | | | | | Photo- | | | | pigment in |
| Photosensitive | | | | | Poly- | | poly- | | | | total solid |
| coloring | Pigment | Part by | | Part by | merizable | Part by | merization | Part by | | Part by | content (% |
| composition | composition | mass | Resin | mass | compound | mass | initiator | mass | Solvent | mass | by mass) |
| RP-51 | R-51 | 300 | PB-6 | 6 | M-1 | 1.8 | I-1 | 3 | S-1 | 100 | 58.8 |
| RP-52 | R-52 | 300 | PB-6 | 6 | M-1 | 1.8 | I-1 | 3 | S-1 | 100 | 58.8 |
| RP-53 | R-53 | 300 | PB-6 | 6 | M-1 | 1.8 | I-1 | 3 | S-1 | 100 | 58.8 |
| RP-54 | R-54 | 300 | PB-6 | 6 | M-1 | 1.8 | I-1 | 3 | S-1 | 100 | 59.3 |
| RP-55 | R-55 | 300 | PB-6 | 6 | M-1 | 1.8 | I-1 | 3 | S-1 | 100 | 54.3 |
| RP-56 | R-56 | 300 | PB-6 | 6 | M-1 | 1.8 | I-1 | 3 | S-1 | 100 | 58.8 |
| RP-57 | R-57 | 300 | PB-6 | 6 | M-1 | 1.8 | I-1 | 3 | S-1 | 100 | 58.8 |
| RP-58 | R-58 | 300 | PB-6 | 6 | M-1 | 1.8 | I-1 | 3 | S-1 | 100 | 60.0 |
| RP-59 | R-59 | 300 | PB-6 | 6 | M-1 | 1.8 | I-1 | 3 | S-1 | 100 | 57.5 |
| RP-60 | R-1 | 300 | PB-6 | 6 | M-2 | 1.8 | I-1 | 3 | S-1 | 100 | 58.8 |
| RP-61 | R-1 | 300 | PB-6 | 6 | M-3 | 1.8 | I-1 | 3 | S-1 | 100 | 58.8 |
| RP-62 | R-1 | 300 | PB-6 | 6 | M-l:M- | 1.8 | I-1 | 3 | S-1 | 100 | 58.8 |
| | | | | | 2 = 50:50 | | | | | | |
| RP-63 | R-1 | 300 | PB-6 | 6 | M-1 | 1.8 | I-2 | 3 | S-1 | 100 | 58.8 |
| RP-64 | R-1 | 300 | PB-6 | 6 | M-1 | 1.8 | I-3 | 3 | S-1 | 100 | 58.8 |
| RP-65 | R-1 | 300 | PB-6 | 6 | M-1 | 1.8 | 14 | 3 | S-1 | 100 | 58.8 |
| RP-66 | R-1 | 300 | PB-6 | 6 | M-1 | 1.8 | I-5 | 3 | S-1 | 100 | 58.8 |
| RP-67 | R-1 | 300 | PB-6 | 6 | M-1 | 1.8 | I-6 | 3 | S-1 | 100 | 58.8 |
| RP-68 | R-1 | 300 | PB-6 | 6 | M-1 | 1.8 | I-7 | 3 | S-1 | 100 | 58.8 |
| RP-69 | R-1 | 300 | PB-6 | 6 | M-1 | 1.8 | I-8 | 3 | S-1 | 100 | 58.8 |
| RP-70 | R-1 | 300 | PB-6 | 6 | M-1 | 1.8 | I-9 | 3 | S-1 | 100 | 58.8 |
| RP-71 | R-1 | 300 | PB-6 | 6 | M-1 | 1.8 | I-10 | 3 | S-1 | 100 | 58.8 |
| RP-72 | R-1 | 300 | PB-6 | 6 | M-1 | 1.8 | I-l:I-3 = | 3 | S-1 | 100 | 58.8 |
| | | | | | | | 50:50 | | | | |
| RP-73 | R-1 | 300 | PB-7 | 6 | M-1 | 1.8 | I-1 | 3 | S-1 | 100 | 58.8 |
| RP-74 | R-1 | 300 | PB-6:PB- | 6 | M-1 | 1.8 | I-1 | 3 | S-1 | 100 | 58.8 |
| | | | 7 = 50:50 | | | | | | | | |
| RP-75 | R-1 | 300 | PB-6 | 6 | M-1 | 1.8 | I-1 | 3 | S-2 | 100 | 58.8 |
| RP-76 | R-1 | 300 | PB-6 | 6 | M-1 | 1.8 | I-1 | 3 | S-l:S- | 100 | 58.8 |
| | | | | | | | | | 2 = 50:50 | | |
| RP-77 | R-60 | 300 | PB-6 | 6 | M-1 | 1.8 | I-1 | 3 | S-1 | 100 | 58.8 |
| RP-78 | R-61 | 300 | PB-6 | 6 | M-1 | 1.8 | I-1 | 3 | S-1 | 100 | 58.8 |
| RP-79 | R-62 | 300 | PB-6 | 6 | M-1 | 1.8 | I-1 | 3 | S-1 | 100 | 58.8 |
| RP-80 | R-63 | 300 | PB-6 | 6 | M-1 | 1.8 | I-1 | 3 | S-1 | 100 | 58.8 |
| RP-81 | R-64 | 300 | PB-6 | 6 | M-1 | 1.8 | I-1 | 3 | S-1 | 100 | 58.8 |
| RP-82 | R-65 | 300 | PB-6 | 6 | M-1 | 1.8 | I-1 | 3 | S-1 | 100 | 58.8 |
| RP-C1 | R-C1 | 300 | PB-6 | 6 | M-1 | 1.8 | I-1 | 3 | S-1 | 100 | 58.8 |
| RP-C2 | R-C2 | 300 | PB-6 | 6 | M-1 | 1.8 | I-1 | 3 | S-1 | 100 | 58.8 |

RP-1 to RP-54, RP-56 to RP-82, RP-C1, and RP-C2 were each Red photosensitive compositions, and RP-55 was a Yellow photosensitive composition. In addition, RP-34 could be suitably used not only for a red pixel but also an orange pixel.

Compounds described in Tables 4 and 5 other than those described above are shown below.

—Resin—

PB-6: solid content: 30% by mass, PGMEA solution, Mw: 30,000, acid value of solid content: 30 mgKOH/g

PB-7: solid content: 30% by mass, PGMEA solution, Mw: 11,000, acid value of solid content: 70 mgKOH/g

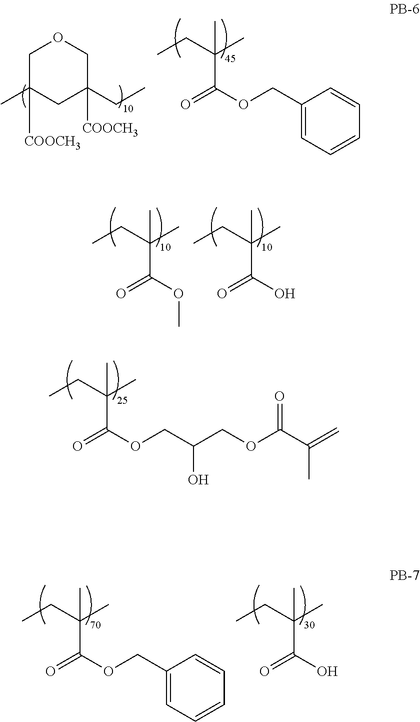

—Polymerizable Compound—

M-1: KAYARAD DPHA (manufactured by Nippon Kayaku Co., Ltd.)

M-2: NK ESTER A-TMMT (manufactured by Shin-Nakamura Chemical Co., Ltd.)

M-3: dipentaerythritol hexaacrylate

—Photopolymerization Initiator—

I-1 to I-10: compounds shown below

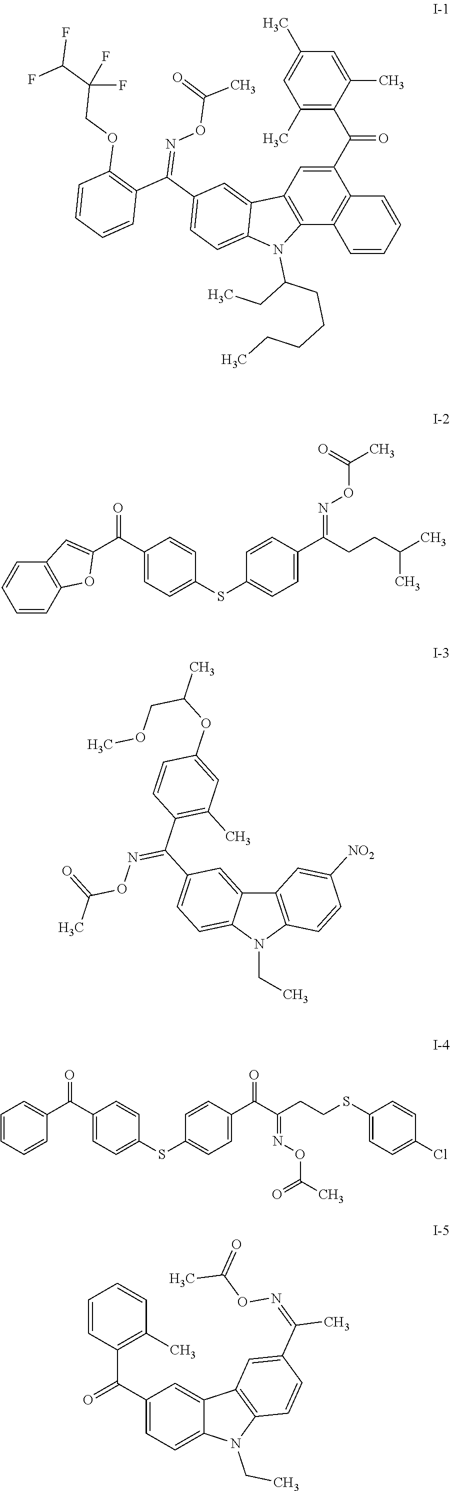 [SMILES: CCCCCC(CC)n1c2ccc(/C(=N\OC(C)=O)c3ccccc3OCC(F)(F)C(F)F)cc2c2cc(C(=O)c3c(C)cc(C)cc3C)c3ccccc3c21 ; CC(=O)O/N=C(\CCC(C)C)c1ccc(Sc2ccc(C(=O)c3cc4ccccc4o3)cc2)cc1 ; CCn1c2ccc(/C(=N\OC(C)=O)c3ccc(OC(C)COC)cc3C)cc2c2cc([N+](=O)[O-])ccc21 ; CC(=O)O/N=C(\CCSc1ccc(Cl)cc1)C(=O)c1ccc(Sc2ccc(C(=O)c3ccccc3)cc2)cc1 ; CCn1c2ccc(C(=O)c3ccccc3C)cc2c2cc(/C(C)=N\OC(C)=O)ccc21]

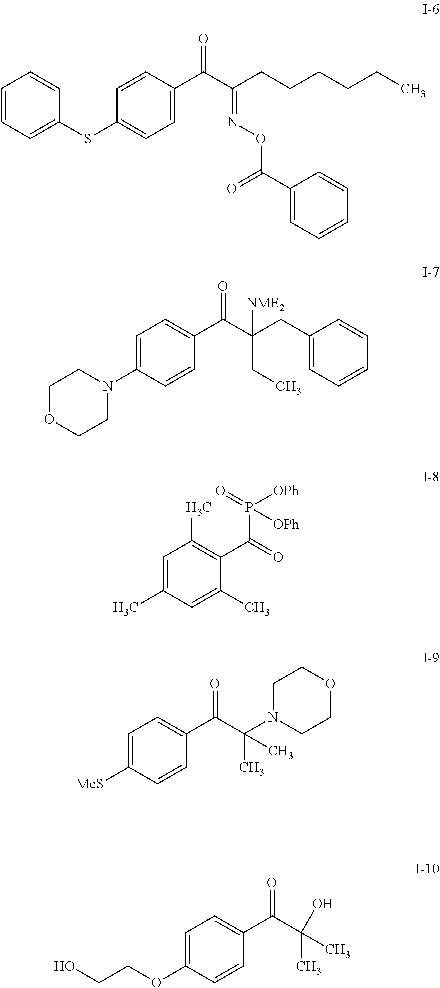

—Solvent—

S-1: propylene glycol monomethyl ether acetate (PGMEA)

S-2: cyclohexanone

<Evaluation Method>

—Evaluation of Adhesiveness—

An 8-inch (20.32 cm) silicon wafer was coated with each photosensitive coloring composition by a spin coating method so that a film thickness after post-baking was 0.5 μm. Next, the silicon wafer was pre-baked using a hot plate at 100° C. for 2 minutes. Next, using an i-ray stepper exposure device FPA-3000 i5+(manufactured by Canon Corporation), exposure was performed with an exposure amount of 200 mJ/cm² through a mask having a Bayer pattern in which a predetermined pixel (pattern) size was formed. As the mask, a mask having a Bayer pattern in which a pixel pattern is formed in a shape of 0.7 μm square, 0.8 μm square, 0.9 μm square, 1.0 μm square, 1.1 μm square, 1.2 μm square, 1.3 μm square, 1.4 μm square, 1.5 μm square, 1.7 μm square, 2.0 μm square, 3.0 μm square, 5.0 μm square, or 10.0 m square was used.

Next, puddle development was performed at 23° C. for 60 seconds using a 0.3% by mass of tetramethylammonium hydroxide (TMAH) aqueous solution. Thereafter, rinsing was performed by a spin shower using pure water. Next, a pattern (pixel) was formed by heating (post-baking) at 200° C. for 5 minutes using a hot plate.

Using a high-resolution FEB measuring device (HITACHI CD-SEM) S9380II (manufactured by Hitachi High-Tech Corporation.), a pattern of 0.7 μm square, 0.8 μm square, 0.9 μm square, 1.0 μm square, 1.1 μm square, 1.2 μm square, 1.3 μm square, 1.4 μm square, 1.5 μm square, 1.7 μm square, 2.0 μm square, 3.0 μm square, 5.0 μm square, or 10.0 μm square was observed, and the minimum pattern size in which the pattern was formed without peeling was defined as a minimum contact line width. It means that the smaller the minimum contact line width, the better the adhesiveness is.

[Evaluation Standard]

A: minimum contact line width was 1.2 μm square or less.

B: minimum contact line width was more than 1.2 μm square and 1.3 μm square or less.

C: minimum contact line width was more than 1.3 μm square and 1.4 μm square or less.

D: minimum contact line width was more than 1.4 μm square and 1.6 μm square or less.

E: minimum contact line width was more than 1.6 μm square.

—Evaluation of Storage Stability—

The viscosity of the photosensitive coloring composition obtained as described above was measured by “RE-85L” manufactured by TOKI SANGYO CO., LTD. After that, the photosensitive coloring composition was left to stand under the conditions of 45° C. and 3 days, and then the viscosity thereof was measured again. Storage stability was evaluated according to the following evaluation standard from a viscosity difference (ΔVis) before and after leaving to stand. It can be said that the smaller the numerical value of the viscosity difference (ΔVis), the better the storage stability. The viscosity of the photosensitive coloring composition was measured in a state in which the temperature was adjusted to 25° C. The evaluation standard was as follows.

[Evaluation Standard]

A: ΔVis was 0.5 mPa·s or less.

B: ΔVis was more than 0.5 mPa·s and 2.0 mPa·s or less.

C: ΔVis was more than 2.0 mPa·s.

—Evaluation of Developability—

CT-4000 (manufactured by Fujifilm Electronic Materials Co., Ltd.) was applied to a silicon wafer by a spin coating method so that a film thickness was 0.1 μm, and the silicon wafer was heated at 220° C. for 1 hour using a hot plate to form a base layer. Each photosensitive coloring composition was applied to this silicon wafer with a base layer by a spin coating method, and then the silicon wafer with a base layer was heated at 100° C. for 2 minutes using a hot plate to obtain a composition layer having a film thickness of 1 μm. Using an i-ray stepper FPA-3000 i5+(manufactured by Canon Inc.), the composition layer was irradiated with light having a wavelength of 365 nm through a mask pattern in which each of the square pixels with a side length of 1.1 μm was arranged on the substrate in a region of 4 mm×3 mm to perform exposure thereon with an exposure amount of 200 mJ/cm². The composition layer after exposure was subjected to puddle development for 60 seconds at 23° C. using a 0.3% by mass of aqueous solution of tetramethylammonium hydroxide. Next, the composition layer was rinsed by spin showering with water and was cleaned with pure water. Thereafter, water droplets were splashed by high-pressure air, and the silicon wafer was naturally dried. Next, post-baking was performed for 300 seconds at 200° C. using a hot plate to form a pattern. The presence or absence of residues between the patterns was observed to evaluate the developability.

The area (non-exposed portion) other than the pattern formation area was observed with a scanning electron microscope (SEM) (magnification: 10,000 times), the number of residues having a diameter of 0.1 μm or more per an area (one area) of 5 μm×5 μm of the non-exposed portion was counted, and the residue was evaluated according to the following evaluation standard.

A: there was no residue per one area.

B: number of residues per one area was less than 10.

C: number of residues per one area was 10 or more and less than 20.

D: number of residues per one area was 20 or more and less than 30.

TABLE 6

| | Photosensitive | | | | | Photosensitive | | | |
|---|---|---|---|---|---|---|---|---|---|
| | coloring | | | | | coloring | | Storage | |
| | composition | Adhesiveness | stability | Developability | | composition | Adhesiveness | stability | Developability |
| Example 1 | RP-1 | A | A | A | Example 43 | RP-43 | B | B | B |
| Example 2 | RP-2 | A | A | A | Example 44 | RP-44 | A | A | A |
| Example 3 | RP-3 | A | A | A | Example 45 | RP-45 | A | A | A |
| Example 4 | RP-4 | A | A | A | Example 46 | RP-46 | A | A | A |
| Example 5 | RP-5 | A | A | A | Example 47 | RP-47 | A | A | B |
| Example 6 | RP-6 | A | A | A | Example 48 | RP-48 | A | A | A |
| Example 7 | RP-7 | A | A | A | Example 49 | RP-49 | A | A | A |
| Example 8 | RP-8 | A | A | A | Example 50 | RP-50 | A | A | A |
| Example 9 | RP-9 | A | A | A | Example 51 | RP-51 | A | A | A |
| Example 10 | RP-10 | A | A | A | Example 52 | RP-52 | A | A | A |
| Example 11 | RP-11 | A | A | A | Example 53 | RP-53 | A | A | A |
| Example 12 | RP-12 | A | A | A | Example 54 | RP-54 | A | A | A |
| Example 13 | RP-13 | A | A | A | Example 55 | RP-55 | A | A | A |
| Example 14 | RP-14 | A | A | A | Example 56 | RP-56 | A | A | A |
| Example 15 | RP-15 | A | A | A | Example 57 | RP-57 | A | A | A |
| Example 16 | RP-16 | A | A | A | Example 58 | RP-58 | A | A | A |
| Example 17 | RP-17 | A | A | A | Example 59 | RP-59 | A | A | A |
| Example 18 | RP-18 | A | B | A | Example 60 | RP-60 | A | A | A |
| Example 19 | RP-19 | A | B | A | Example 61 | RP-61 | A | A | A |
| Example 20 | RP-20 | B | A | B | Example 62 | RP-62 | A | A | A |
| Example 21 | RP-21 | A | B | A | Example 63 | RP-63 | A | A | A |
| Example 22 | RP-22 | A | A | A | Example 64 | RP-64 | A | A | A |
| Example 23 | RP-23 | A | A | A | Example 65 | RP-65 | A | A | A |
| Example 24 | RP-24 | A | A | A | Example 66 | RP-66 | A | A | A |
| Example 25 | RP-25 | A | A | A | Example 67 | RP-67 | A | A | A |
| Example 26 | RP-26 | B | A | B | Example 68 | RP-68 | A | A | A |
| Example 27 | RP-27 | C | A | C | Example 69 | RP-69 | A | A | A |
| Example 28 | RP-28 | A | A | A | Example 70 | RP-70 | A | A | A |
| Example 29 | RP-29 | A | A | A | Example 71 | RP-71 | A | A | A |
| Example 30 | RP-30 | B | A | B | Example 72 | RP-72 | A | A | A |
| Example 31 | RP-31 | C | A | C | Example 73 | RP-73 | A | A | A |
| Example 32 | RP-32 | A | A | A | Example 74 | RP-74 | A | A | A |
| Example 33 | RP-33 | A | A | A | Example 75 | RP-75 | A | A | A |
| Example 34 | RP-34 | A | A | A | Example 76 | RP-76 | A | A | A |
| Example 35 | RP-35 | A | A | A | Example 77 | RP-77 | D | A | C |
| Example 36 | RP-36 | A | A | A | Example 78 | RP-78 | B | A | B |
| Example 37 | RP-37 | A | A | A | Example 79 | RP-79 | A | A | A |
| Example 38 | RP-38 | A | A | A | Example 80 | RP-80 | A | A | A |
| Example 39 | RP-39 | A | B | A | Example 81 | RP-81 | B | B | C |
| Example 40 | RP-40 | B | B | B | Example 82 | RP-82 | B | B | C |
| Example 41 | RP-41 | A | A | A | Comparative | RP-C1 | E | C | D |
| | | | | | Example 1 | | | | |
| Example 42 | RP-42 | A | A | A | Comparative | RP-C2 | E | C | D |
| | | | | | Example 2 | | | | |

As shown in Table 6, compared to the photosensitive coloring compositions of Comparative Examples 1 and 2, the photosensitive coloring compositions of Examples 1 to 82 had excellent adhesiveness to the obtained cured substance.

In addition, as shown in Table 6, the photosensitive coloring compositions of Examples 1 to 82 were also excellent in storage stability and developability.

### Examples 101 to 154 and Examples 156 to 182

A silicon wafer was coated with a Green composition using a spin coating method so that a thickness of a film after film formation was 1.0 μm. Next, the silicon wafer was heated using a hot plate at 100° C. for 2 minutes. Next, using an i-ray stepper exposure device FPA-3000 i5+(manufactured by Canon Inc.), exposure was performed at 1,000 mJ/cm² through a mask having a dot pattern of 2 μm square. Next, puddle development was performed at 23° C. for 60 seconds using a 0.3% by mass of tetramethylammonium hydroxide (TMAH) aqueous solution. Next, the coating film was rinsed by spin showering and was cleaned with pure water. Next, the Green composition was patterned on the silicon wafer by heating at 200° C. for 5 minutes using a hot plate. Likewise, a Red composition and a Blue composition were sequentially patterned to form red, green, and blue colored patterns (Bayer pattern).

In Examples 101 to 154 and Examples 156 to 182, as the Red composition, the photosensitive coloring compositions prepared in Examples 1 to 54 and Examples 56 to 82 were used, respectively.

The Green composition and Blue composition other than the above-described photosensitive coloring composition will be described later.

The Bayer pattern refers to a pattern, as described in the specification of U.S. Pat. No. 3,971,065A, in which a 2×2 array of color filter element having one Red element, two Green elements, and one Blue element is repeated.

The obtained color filter was incorporated into a solid-state imaging element according to a known method. In a case where any of the photosensitive coloring compositions obtained in Examples 1 to 76 was used, it was confirmed that the solid-state imaging element had excellent adhesiveness in the cured film, and that a solid-state imaging element having suitable image recognition ability was obtained.

The Green composition and Blue composition other than the above-described photosensitive coloring compositions used in Examples 101 to 154 and Examples 156 to 182 are as follows.

—Green Composition—

The following components were mixed and stirred, and the obtained mixture was filtered through a nylon filter (manufactured by Nihon Pall Corporation) having a pore size of 0.45 μm to prepare a Green composition.

Green pigment dispersion liquid: 73.7 parts by mass

Resin 4 (40% by mass PGMEA solution): 0.3 parts by mass

Polymerizable compound 1: 1.2 parts by mass

Photopolymerization initiator 1: 0.6 parts by mass

Surfactant 1: 4.2 parts by mass

Ultraviolet absorber (UV-503, manufactured by Daito Chemical Co., Ltd.): 0.5 parts by mass

PGMEA: 19.5 parts by mass

—Blue Composition—

The following components were mixed and stirred, and the obtained mixture was filtered through a nylon filter (manufactured by Nihon Pall Corporation) having a pore size of 0.45 μm to prepare a Blue composition.

Blue pigment dispersion liquid: 44.9 parts by mass

Resin 4 (40% by mass PGMEA solution): 2.1 parts by mass

Polymerizable compound 1: 1.5 parts by mass

Polymerizable compound 4: 0.7 parts by mass

Photopolymerization initiator 1: 0.8 parts by mass

Surfactant 1: 4.2 parts by mass

PGMEA: 45.8 parts by mass

Raw materials used in the Green composition, Red composition, and Blue composition are as follows.

Green Pigment Dispersion Liquid

A mixed solution consisting of 6.4 parts by mass of C. I. Pigment Green 36, 5.3 parts by mass of C. I. Pigment Yellow 150, 5.2 parts by mass of a dispersant (DISPERBYK-161, manufactured by BYK Chemie), and 83.1 parts by mass of PGMEA was mixed and dispersed using a beads mill (zirconia beads; diameter: 0.3 mm) for 3 hours to prepare a pigment dispersion liquid. Next, using a high-pressure disperser NANO-3000-10 (manufactured by Nippon BEE Chemical Co., Ltd.) equipped with a pressure reducing mechanism, the pigment dispersion liquid was further dispersed under a pressure of 2,000 kg/cm² at a flow rate of 500 g/min. This dispersion treatment was repeated 10 times. As a result, a Green pigment dispersion liquid was obtained.

Blue Pigment Dispersion Liquid

A mixed solution consisting of 9.7 parts by mass of C. I. Pigment Blue 15:6, 2.4 parts by mass of C. I. Pigment Violet 23, 5.5 parts of a dispersant (DISPERBYK-161, manufactured by BYK Chemie), and 82.4 parts of PGMEA was mixed and dispersed using a beads mill (zirconia beads; diameter: 0.3 mm) for 3 hours to prepare a pigment dispersion liquid. Next, using a high-pressure disperser NANO-3000-10 (manufactured by Nippon BEE Chemical Co., Ltd.) equipped with a pressure reducing mechanism, the pigment dispersion liquid was further dispersed under a pressure of 2,000 kg/cm² at a flow rate of 500 g/min. This dispersion treatment was repeated 10 times, thereby obtaining a Blue pigment dispersion liquid.
Polymerizable compound 1: KAYARAD DPHA (mixture of dipentaerythritol hexaacrylate and dipentaerythritol pentaacrylate, manufactured by Nippon Kayaku Co., Ltd.)
Polymerizable compound 4: compound having the following structure

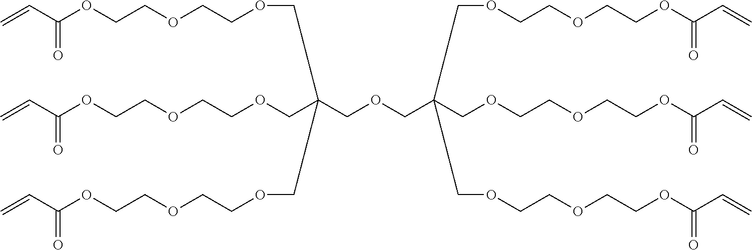 [SMILES: C=CC(=O)OCCOCCOCC(COCCOCCOC(=O)C=C)(COCCOCCOC(=O)C=C)COCC(COCCOCCOC(=O)C=C)(COCCOCCOC(=O)C=C)COCCOCCOC(=O)C=C]

Resin 4: resin having the following structure (acid value: 70 mgKOH/g, Mw=11,000; a ratio in a constitutional unit is a molar ratio)

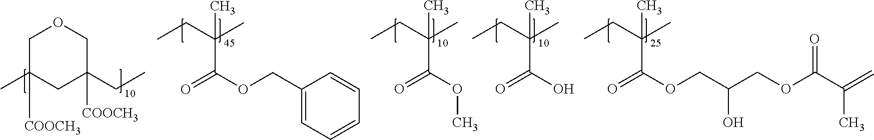

Photopolymerization initiator 1: IRGACURE OXE01 (1-[4-(phenylthio)phenyl]-1,2-octanedione-2-(O-benzoyloxime), manufactured by BASF SE)
Surfactant 1: 1% by mass PGMEA solution of the following mixture (Mw: 14,000; in the following formula, the unit of % (62% and 38%) indicating the proportion of a constitutional unit is % by mass)

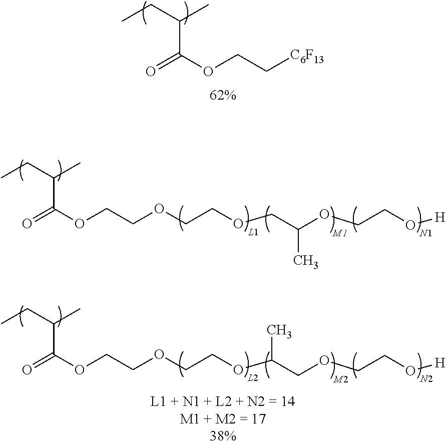

The disclosure of Japanese Patent Application No. 2020-030706 filed on Feb. 26, 2020 is incorporated in the present specification by reference.

All documents, patent applications, and technical standards described in the present specification are incorporated herein by reference to the same extent as in a case of being specifically and individually noted that individual documents, patent applications, and technical standards are incorporated by reference.

What is claimed is:

1. A photosensitive coloring composition comprising:
a pigment; and
a diketopyrrolopyrrole compound A represented by Formula 1,
wherein, in a case where a molar content of the diketopyrrolopyrrole compound A represented by Formula 1 in the photosensitive coloring composition is denoted by m^{A} and a molar content of a diketopyrrolopyrrole compound B represented by Formula 1 is denoted by m^{B}, a value of m^{A}/(m^{A}+m^{B}) is 10 mol % to 100 mol %, and
a content of the pigment is 35% by mass or more with respect to a total solid content of the photosensitive coloring composition, 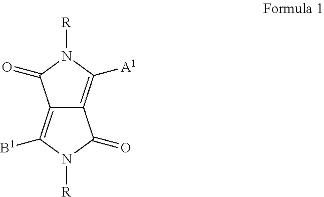
in Formula 1,
diketopyrrolopyrrole compound A: A¹ represents a monovalent organic group having an acidic functional group or a basic functional group, B¹ represents a monovalent organic group not having an acidic functional group and a basic functional group, and R's each independently represent a hydrogen atom or a monovalent substituent,
diketopyrrolopyrrole compound B: A¹ and B¹ represent monovalent organic groups having an acidic functional group or a basic functional group, A¹ and B¹ may be the same or different from each other, and R's each independently represent a hydrogen atom or a monovalent substituent.

2. The photosensitive coloring composition according to claim 1,
wherein the diketopyrrolopyrrole compound A includes an asymmetric diketopyrrolopyrrole compound represented by Formula 2, 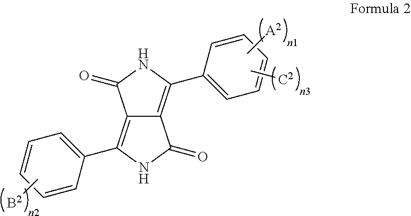
in Formula 2, A²'s each independently represent a monovalent organic group having an acidic functional group or a basic functional group, B²'s each independently represent a monovalent organic group not having an acidic functional group and a basic functional group, C²'s each independently represent a monovalent organic group not having an acidic functional group and a basic functional group, n1 represents an integer of 1 to 5, n2 represents an integer of 0 to 5, n3 represents an integer of 0 to 4, and a phenyl group to which A² and C² are bonded and a phenyl group to which B² is bonded are different groups.

3. The photosensitive coloring composition according to claim 1,
wherein the value of m^{A}/(m^{A}+m^{B}) is more than 90 mol % and 100 mol % or less.

4. The photosensitive coloring composition according to claim 1,
wherein A¹ is a monovalent organic group having a basic functional group.

5. The photosensitive coloring composition according to claim 1,
wherein the pigment includes a diketopyrrolopyrrole pigment other than the compound represented by Formula 1.

6. The photosensitive coloring composition according to claim 1,
wherein the pigment includes a diketopyrrolopyrrole red pigment other than the compound represented by Formula 1.

7. The photosensitive coloring composition according to claim 1,
wherein the pigment includes a diaryldiketopyrrolopyrrole red pigment other than the compound represented by Formula 1, which has an electron donating group on an aromatic ring.

8. The photosensitive coloring composition according to claim 1,
wherein the content of the pigment is 50% by mass or more with respect to the total solid content of the photosensitive coloring composition.

9. The photosensitive coloring composition according to claim 1,
wherein a mass ratio of a content M^{P} of the pigment and a content M^{A} of the diketopyrrolopyrrole compound A in the photosensitive coloring composition is M^{P}/M^{A}=95/5 to 50/50.

10. The photosensitive coloring composition according to claim 1, further comprising:
a resin.

11. The photosensitive coloring composition according to claim 10,
wherein the resin includes a resin having an acidic functional group.

12. The photosensitive coloring composition according to claim 1, further comprising:
a polymerizable compound; and
a photopolymerization initiator.

13. A cured substance obtained by curing the photosensitive coloring composition according to claim 1.

14. A color filter comprising:
the cured substance according to claim 13.

15. A solid-state imaging element comprising:
the color filter according to claim 14.

16. An image display device comprising:
the color filter according to claim 14.

17. An asymmetric diketopyrrolopyrrole compound represented by Formula 3, 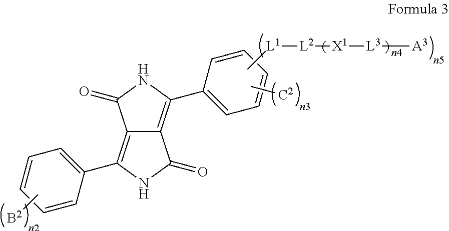
in Formula 3, A³'s each independently represent an acidic functional group or a basic functional group, B²'s each independently represent a monovalent organic group not having an acidic functional group and a basic functional group, C²'s each independently represent a monovalent organic group not having an acidic functional group and a basic functional group, X¹'s each independently represent an ether bond, a thioether bond, a sulfonamide bond, or a urea bond, L¹'s each independently represent a single bond or an ether bond, L²'s and L³'s each independently represent an alkylene group, n2 represents an integer of 0 to 5, n3 represents an integer of 0 to 4, n4's each independently represent 0 or 1, n5 represents an integer of 1 to 5, a group having A³ at a terminal, a phenyl group to which C² is bonded, and a phenyl group to which B² is bonded are different groups, and in a case where L¹ is an ether bond, B² is an electron donating group not having an acidic functional group and a basic functional group and n2 represents an integer of 1 to 5.
